(19) United States
(12) Patent Application Publication
(10) Pub. No.: US 2024/0390439 A1
(43) Pub. Date: Nov. 28, 2024

(54) COMPOSITIONS AND METHODS OF SENECA VALLEY VIRUS (SVV) RELATED CANCER THERAPY

(71) Applicant: ElevateBio Technologies, Inc., Waltham, MA (US)

(72) Inventors: Lorena LERNER, Andover, MA (US); Edward M. KENNEDY, Andover, MA (US); Jonathan Michael James DERRY, Hansville, WA (US); Christophe QUÉVA, Andover, MA (US); Jeffrey David BRYANT, Cambridge, MA (US)

(21) Appl. No.: 18/733,446

(22) Filed: Jun. 4, 2024

Related U.S. Application Data
(63) Continuation of application No. PCT/US2022/080898, filed on Dec. 5, 2022.
(60) Provisional application No. 63/286,248, filed on Dec. 6, 2021.

(51) Int. Cl. A61K 35/768; A61K 9/51; A61P 35/00; C12Q 1/6886; G16B 25/10; G16B 40/20

(52) U.S. Cl. CPC A61K 35/768; A61K 9/5123; A61P 35/00; C12Q 1/6886; G16B 25/10; G16B 40/20; C12Q 2600/106; C12Q 2600/158

(57) ABSTRACT

The present disclosure relates to methods of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer. Methods of selecting subjects for treatment are also provided herein.

### CROSS-REFERENCE TO RELATED APPLICATIONS

This application is a Continuation of International Application No. PCT/US2022/080898, filed Dec. 5, 2022, which claims the benefit of U.S. Provisional Application No. 63/286,248, filed Dec. 6, 2021, the content of each of which is herein incorporated by reference in its entirety.

### REFERENCE TO AN ELECTRONIC SEQUENCE LISTING

The content of the electronic sequence listing (ELVT_024_O1US_SubSeqList_ST26.xml; Size: 33,815 bytes; and Date of Creation: Jun. 20, 2024) is herein incorporated by reference in its entirety.

### FIELD

The present disclosure generally relates to the fields of oncolytic viruses and cancer therapeutics. More specifically, the present disclosure relates to determining the sensitivity of a cancer to treatment with an oncolytic virus based on the expression level of one or more genes. The disclosure further relates to the treatment and prevention of proliferative disorders such as cancer.

### BACKGROUND

Oncolytic viruses are replication-competent viruses with lytic life-cycle able to infect and lyse tumor cells. Direct tumor cell lysis results not only in cell death, but also the generation of an adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

Seneca Valley Virus (SVV) is an oncolytic picornavirus, which has been reported to selectively infects cancers with neuroendocrine features. SVV is notable for its small size, rapid doubling time, high selectivity for neuroendocrine cancer cells. SVV may be administered to patients in a number of forms, such as in its native form or in the form of SVV viral RNA encapsulated by a lipid nanoparticle (LNP).

Clinical development of oncolytic virus-based cancer treatments poses several challenges, one of which is a lack of means to differentiate cancer cells that are susceptible to virus infection (e.g., virus-sensitive cancer cells) from those that are resistant to virus infection (e.g., virus-resistant cancer cells). Identification of cancers that are susceptible to oncolytic virus infection will enhance the efficacy of these oncolytic virus-based treatments.

There remains a need in the art for methods and kits related to determining the sensitivity of cancers to oncolytic viruses, such as SVV, which would greatly facilitate patient selection and improve the efficacy of oncolytic virus-related cancer therapies. The present disclosure provides such methods, related kits, and more.

### SUMMARY

In one aspect, the disclosure provides methods of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer, and wherein the one or more genes comprise at least one of the genes listed in one of Tables 1-14 or a combination thereof.

In one aspect, the disclosure provides methods of treating a cancer in a subject in need thereof, comprising: (a) determining the expression level of one or more genes in the cancer; (b) classifying the cancer as sensitive to Seneca Valley Virus (SVV) infection based on the expression level of the one or more genes as determined in (a); and (c) administering a therapeutically effective amount of a Seneca Valley Virus (SVV) or a polynucleotide encoding the SVV viral genome to the subject if the cancer is classified as sensitive to SVV infection in step (b), wherein the one or more genes comprise at least one of the genes listed in one of Tables 1-14 or a combination thereof.

In one aspect, the disclosure provides methods of evaluating the sensitivity of a cancer to Seneca Valley Virus (SVV) infection, comprising determining the expression level of one or more genes in the cancer, wherein the one or more genes comprise at least one of the genes listed in one of Tables 1-14 or a combination thereof, and classifying the cancer as sensitive or resistant to SVV infection based on the expression level of the one or more genes. In some embodiments, the method further comprises administering a SVV or a polynucleotide encoding the SVV viral genome to the cancer.

In one aspect, the disclosure provides methods of selecting a subject suffering from a cancer for treatment with a Seneca Valley Virus (SVV) or a polynucleotide encoding the SVV viral genome, comprising: (a) determining the expression level of one or more genes in the cancer, wherein the one or more genes comprise at least one of the genes listed in one of Tables 1-14 or a combination thereof; (b) classifying the cancer as sensitive to SVV infection based on the expression level of the one or more genes as determined in (a); and (c) selecting the subject for treatment with the SVV or the polynucleotide encoding the SVV viral genome if the cancer is classified as sensitive to SVV infection in (b). In some embodiments, the method further comprises: (d) administering the SVV or the polynucleotide encoding the SVV viral genome to the selected subject.

In one aspect, the disclosure provides methods of determining the expression level of one or more genes in a cancer, wherein the one or more genes comprise at least one of the genes listed in one of Tables 1-14 or a combination thereof.

In some embodiments, the one or more genes comprise at least one gene selected from one of Tables 2-7. In some embodiments, the one or more genes comprise at least 2,3,4,5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 genes selected from one of Tables 1-14.

In some embodiments, the one or more genes have a frequency of at least 5% in Table 2 or 3. In some embodiments, the one or more genes have a frequency of at least 10%, 15%, 20%, 25%, 30%, 35%, 40%, 45%, 50%, 55%, 60%, 65%, 70%, 75%, or 80% in Table 2 or 3. In some embodiments, the one or more genes comprise all genes having a frequency of at least 40% in Table 2 or 3. In some embodiments, the one or more genes comprise all genes having a frequency of at least 10%, 15%, 20%, 25%, 30%, 35%, 40%, 45%, 50%, 55%, 60%, 65%, 70%, 75%, or 80% in Table 2 or 3.

In some embodiments, the frequency of the gene is the number of runs in which the gene is selected in an elastic net modeling divided by the total number of runs of the elastic net modeling.

In some embodiments, the one or more genes comprise all genes in one of Tables 2-7, or all genes in Tables 8-9, or all genes in Tables 10-11. In some embodiments, the one or more genes comprise all genes in Table 3.

In some embodiments, the one or more genes comprise two genes selected from one of combinations #1 to #55 of Table 13, three genes selected from one of combinations #56 to #220 of Table 13, four genes selected from one of combinations #221 to #550 of Table 13, or five genes selected from one of combinations #551 to #1012 of Table 13.

In some embodiments, the one or more genes comprise two genes selected from one of combinations #1 to #55 of Table 14, three genes selected from one of combinations #56 to #220 of Table 14, four genes selected from one of combinations #221 to #550 of Table 14, or five genes selected from one of combinations #551 to #1012 of Table 14.

In some embodiments, the one or more genes comprise at least one gene encoding a protein with adaptive immunity function selected from TAP1, MICB, HLA-B, HLA-C, HLA-E, HLA-H, and CTSS.

In some embodiments, the one or more genes comprise at least one gene encoding a protein with immune response function selected from CLEC2D, TNFRSF14, CD226, TNFAIP3, ANXA1, GSDMD, IKBKE, MAPK13, PARP9, TMEM9B, and UNC93B1.

In some embodiments, the one or more genes comprise at least one gene encoding a protein with cell adhesion/migration function selected from PPFIA4, LRFN5, ASTN1, MYL12A, CLDN5, CD9, SNED1, ITGA4, B4GALT1, and VWA1.

In some embodiments, the one or more genes comprise at least one gene encoding a protein with cellular RNA processing function selected from YBX2, EXOSC3, TAF1B, and USB1.

In some embodiments, the one or more genes comprise at least one gene encoding a protein with intracellular transportation function selected from SCG3, CPLX1, SCAMPI, SYTL2, CTAGE8, SLC52A1, HIP1, COPB1, and MYO5B.

In some embodiments, the one or more genes comprise at least one gene encoding a protein with DNA repair function selected from TYR, FAAP20, and FAM111A.

In some embodiments, the one or more genes comprise at least one gene encoding a protein with G-protein signaling function selected from GNAO1, PLCG2, ARHGEF35, ARHGEF16, RAPGEF3, DENND2D, and ADCY6.

In some embodiments, the one or more genes comprise at least one gene encoding a protein with neurotransmission function selected from SYN2, NSMF, CHRNA1, KCNT2, ATP2B2, TTYH2, CNRIP1, FAM155B, NTNG2, and AGRN.

In some embodiments, the one or more genes comprise at least one gene encoding a transcription factor selected from ETV7, HOXC11, SOX5, NHLH2, MYT1L, GRHL2, STAT6, DACH1, and ARID5A.

In some embodiments, the one or more genes comprise at least one gene encoding a protein with ubiquitination function selected from GID4, RNF112, and UBR1.

In some embodiments, the one or more genes comprise at least one gene selected from the group consisting of HLA-C, STAT6, TMCO4, CNRIP1, DCAF13, PRDM8, DACH1, and IKBKE. In some embodiments, the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE.

In some embodiments, the one or more genes comprise at least one gene selected from the group consisting of HLA-C, STAT6, TMCO4, and CNRIP1. In some embodiments, the one or more genes comprise 2, 3, or 4 genes selected from the group consisting of HLA-C, STAT6, TMCO4, and CNRIP1.

In some embodiments, the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7. In some embodiments, the one or more genes comprise at least one gene selected from the group consisting of HLA-C, CNRIP1, JPH1, TMCO4, STAT6, DENND2D, and ETV7. In some embodiments, the one or more genes comprise at least 2, 3, 4, 5, 6, or 7 genes selected from the group consisting of HLA-C, CNRIP1, JPH1, TMCO4, STAT6, DENND2D, and ETV7. In some embodiments, the one or more genes comprise at least one gene selected from the group consisting of HLA-C, CNRIP1, JPH1, and TMCO4. In some embodiments, the one or more genes comprise 2, 3, or 4 genes selected from the group consisting of HLA-C, CNRIP1, JPH1, and TMCO4.

In some embodiments, the one or more genes comprise HLA-C.

In some embodiments, the one or more genes do not comprise ANTXR1.

In some embodiments, the one or more genes do not comprise IFI35.

In some embodiments, the increased expression of the one or more upregulated genes in one of Tables 2-7, 8 and 10 is indicative of increased SVV sensitivity. In some embodiments, the expression of the one or more upregulated genes is increased by at least 20%, at least 30%, at least 40%, at least 50%, at least 70%, at least 1-fold, at least 2-fold, at least 3-fold, at least 5-fold, or at least 10-fold, compared to a reference gene expression level.

In some embodiments, the reduced expression of the one or more downregulated genes in one of Tables 2-7, 9 and 11 is indicative of increased SVV sensitivity. In some embodiments, the expression of the one or more downregulated genes is decreased by at least 10%, at least 20%, at least 30%, at least 40%, at least 50%, at least 60%, at least 70%, at least 80%, at least 90%, at least 95%, at least 98%, or at least 99%, compared to a reference gene expression level.

In some embodiments, the reference gene expression level is a pre-determined value based on the expression level of the one or more genes in a non-cancerous cell, the expression level of the one or more genes in a reference set of non-cancerous samples, and/or the expression level of the one or more genes in a reference set of cancer samples with known sensitivity to SVV infection.

In some embodiments, the polynucleotide is a recombinant RNA molecule. In some embodiments, the polynucleotide encoding the SVV viral genome is encapsulated in a particle. In some embodiments, the particle is a lipid nanoparticle.

In some embodiments, the expression level of the one or more genes is mRNA expression level. In some embodiments, determining the mRNA expression level comprises performing quantitative real time reverse transcriptase polymerase chain reaction (qRT-PCR), PCR, RNAseq, microarray, gene chip, nCounter Gene Expression Assay, Serial Analysis of Gene Expression (SAGE), Rapid Analysis of Gene Expression (RAGE), nuclease protection assays, Northern blotting, nucleic acid hybridization, or any other equivalent gene expression detection techniques.

In some embodiments, the expression level of the one or more genes is protein expression level. In some embodiments, the protein expression level is determined by antibody-based testing, immunoassay, radioimmunoassay (RIA), immunohistochemistry, immunofluorescence, chemiluminescence, phosphorescence, proteomics techniques, surface plasmon resonance (SPR), mass spectrometry, protein microarray, or any other equivalent protein expression detection techniques.

In some embodiments, the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, bladder cancer, and marginal zone lymphoma (MZL).

In some embodiments, the cancer is a neuroendocrine cancer.

In some embodiments, the cancer is selected from small cell lung cancer (SCLC), large cell neuroendocrine carcinoma (LCNEC), metastatic liver cancer, neuroendocrine-positive prostate cancer (e.g., treatment-emergent small-cell neuroendocrine prostate cancer (t-SCNC)), and Merkel cell carcinoma (MCC).

In some embodiments, the cancer is neuroendocrine-positive (NE+) metastatic castration-resistant prostate cancer (mCRPC).

In some embodiments, the cancer is small cell lung cancer (SCLC). In some embodiments, the cancer is NeuroD1+SCLC.

In some embodiments, the method comprises administering a therapeutic agent selected from an immune checkpoint inhibitor, an engineered immune cell comprising an engineered antigen receptor, a JAK/STAT inhibitor, an mTOR inhibitor, an interferon (IFN) pathway inhibitor, and an HDAC inhibitor. In some embodiments, the immune checkpoint inhibitor is a PD-1 inhibitor or a PD-L1 inhibitor.

In some embodiments, the subject is a mouse, a rat, a rabbit, a cat, a dog, a horse, a non-human primate, or a human.

In some embodiments, the method comprises obtaining a sample of the cancer for determining the expression level of the one or more genes in the cancer.

In some embodiments, a sample of the cancer is used for determining the expression level of the one or more genes, and wherein the sample is a formalin-fixed, paraffin-embedded (FFPE) sample, a fresh tissue sample, a frozen tissue sample, a tumor biopsy sample, an exosome, a wash fluid, a cell pellet, or a bodily fluid. In some embodiments, a sample of the cancer is used for determining the expression level of the one or more genes, and wherein the sample comprises circulating tumor cells (CTCs) or cell-free RNA (cfRNA).

In some embodiments, the cancer has been treated with one or more therapeutic agents. In some embodiments, the cancer has relapsed after the treatment of the therapeutic agent. In some embodiments, the therapeutic agent is a chemotherapeutic agent, a checkpoint kinase inhibitor, or a PARP inhibitor. In some embodiments, the therapeutic agent is a platinum-based drug. In some embodiments, the therapeutic agent is Cisplatin.

In one aspect, the disclosure provides kits comprising reagents for determining the expression level of one or more genes in a sample from a subject in need of, wherein the one or more genes comprise any one of the genes listed in one of Tables 1-14 or a combination thereof.

In some embodiments, the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 genes selected from one of Tables 1-14.

In some embodiments, the one or more genes have a frequency of at least 5%, 10%, 15%, 20%, 25%, 30%, 35%, 40%, 45%, 50%, 55%, 60%, 65%, 70%, 75%, or 80% in Table 2 or 3. In some embodiments, the one or more genes comprise all genes having a frequency of at least 40% in Table 2 or 3. In some embodiments, the one or more genes comprise all genes having a frequency of at least 10%, 15%, 20%, 25%, 30%, 35%, 40%, 45%, 50%, 55%, 60%, 65%, 70%, 75%, or 80% in Table 2 or 3.

In some embodiments, the frequency of the gene is the number of runs in which the gene is selected in an elastic net modeling divided by the total number of runs of the elastic net modeling.

In some embodiments, the one or more genes comprise all genes in one of Tables 2-7, or all genes in Tables 8-9, or all genes in Tables 10-11. In some embodiments, the one or more genes comprise all genes in Table 3.

In some embodiments, the one or more genes comprise two genes selected from one of combinations #1 to #55 of Table 13, three genes selected from one of combinations #56 to #220 of Table 13, four genes selected from one of combinations #221 to #550 of Table 13, or five genes selected from one of combinations #551 to #1012 of Table 13.

In some embodiments, the one or more genes comprise two genes selected from one of combinations #1 to #55 of Table 14, three genes selected from one of combinations #56 to #220 of Table 14, four genes selected from one of combinations #221 to #550 of Table 14, or five genes selected from one of combinations #551 to #1012 of Table 14.

In some embodiments, the one or more genes comprise at least one gene encoding a protein with adaptive immunity function selected from TAP1, MICB, HLA-B, HLA-C, HLA-E, HLA-H, and CTSS.

In some embodiments, the one or more genes comprise at least one gene encoding a protein with immune response function selected from CLEC2D, TNFRSF14, CD226, TNFAIP3, ANXA1, GSDMD, IKBKE, MAPK13, PARP9, TMEM9B, and UNC93B1.

In some embodiments, the one or more genes comprise at least one gene encoding a protein with cell adhesion/migration function selected from PPFIA4, LRFN5, ASTN1, MYL12A, CLDN5, CD9, SNED1, ITGA4, B4GALT1, and VWA1.

In some embodiments, the one or more genes comprise at least one gene encoding a protein with cellular RNA processing function selected from YBX2, EXOSC3, TAF1B, and USB1.

In some embodiments, the one or more genes comprise at least one gene encoding a protein with intracellular transportation function selected from SCG3, CPLX1, SCAMPI, SYTL2, CTAGE8, SLC52A1, HIP1, COPB1, and MYO5B.

In some embodiments, the one or more genes comprise at least one gene encoding a protein with DNA repair function selected from TYR, FAAP20, and FAM111A.

In some embodiments, the one or more genes comprise at least one gene encoding a protein with G-protein signaling function selected from GNAO1, PLCG2, ARHGEF35, ARHGEF16, RAPGEF3, DENND2D, and ADCY6.

In some embodiments, the one or more genes comprise at least one gene encoding a protein with neurotransmission function selected from SYN2, NSMF, CHRNA1, KCNT2, ATP2B2, TTYH2, CNRIP1, FAM155B, NTNG2, and AGRN.

In some embodiments, the one or more genes comprise at least one gene encoding a transcription factor selected from ETV7, HOXC11, SOX5, NHLH2, MYT1L, GRHL2, STAT6, DACH1, and ARID5A.

In some embodiments, the one or more genes comprise at least one gene encoding a protein with ubiquitination function selected from GID4, RNF112, and UBR1.

In some embodiments, the one or more genes comprise at least one gene selected from the group consisting of HLA-C, STAT6, TMCO4, CNRIP1, DCAF13, PRDM8, DACH1, and IKBKE. In some embodiments, the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE.

In some embodiments, the one or more genes comprise at least one gene selected from the group consisting of HLA-C, STAT6, TMCO4, and CNRIP1. In some embodiments, the one or more genes comprise 2, 3, or 4 genes selected from the group consisting of HLA-C, STAT6, TMCO4, and CNRIP1.

In some embodiments, the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7.

In some embodiments, the one or more genes comprise at least one gene selected from the group consisting of HLA-C, CNRIP1, JPH1, TMCO4, STAT6, DENND2D, and ETV7. In some embodiments, the one or more genes comprise at least 2, 3, 4, 5, 6, or 7 genes selected from the group consisting of HLA-C, CNRIP1, JPH1, TMCO4, STAT6, DENND2D, and ETV7.

In some embodiments, the one or more genes comprise at least one gene selected from the group consisting of HLA-C, CNRIP1, JPH1, and TMCO4. In some embodiments, the one or more genes comprise 2, 3, or 4 genes selected from the group consisting of HLA-C, CNRIP1, JPH1, and TMCO4.

In some embodiments, the one or more genes comprise HLA-C.

In some embodiments, the one or more genes do not comprise ANTXR1.

In some embodiments, the one or more genes do not comprise IFI35.

In some embodiments, the kit comprises the reagents for determining the mRNA expression level of the one or more genes. In some embodiments, the reagents comprises reagents for performing quantitative real time reverse transcriptase polymerase chain reaction (qRT-PCR), PCR, RNAseq, microarray, gene chip, nCounter Gene Expression Assay, Serial Analysis of Gene Expression (SAGE), Rapid Analysis of Gene Expression (RAGE), nuclease protection assays, Northern blotting, nucleic acid hybridization, or any other equivalent gene expression detection techniques.

In some embodiments, the kit comprises the reagents for determining the protein expression level of the one or more genes. In some embodiments, the reagents comprises reagents for performing antibody-based testing, immunoassay, radioimmunoassay (RIA), immunohistochemistry, immunofluorescence, chemiluminescence, phosphorescence, proteomics techniques, surface plasmon resonance (SPR), mass spectrometry, protein microarray, or any other equivalent protein expression detection techniques.

In some embodiments, the sample is a formalin-fixed, paraffin-embedded (FFPE) sample, a fresh tissue sample, a frozen tissue sample, a tumor biopsy sample, an exosome, a wash fluid, a cell pellet, a bodily fluid, a circulating tumor cells (CTCs) sample, or a cell-free RNA (cfRNA) sample. In some embodiments, the sample is a cancer sample and wherein the kit is for valuating the sensitivity of the cancer to SVV infection.

In some embodiments, the kit is for use in combination with a composition comprising a Seneca Valley Virus (SVV) or a polynucleotide encoding the SVV viral genome for treating a cancer in the subject.

In some embodiments, the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, bladder cancer, and marginal zone lymphoma (MZL).

In some embodiments, the cancer is a neuroendocrine cancer. In some embodiments, the cancer is selected from small cell lung cancer (SCLC), large cell neuroendocrine carcinoma (LCNEC), metastatic liver cancer, neuroendocrine-positive prostate cancer (e.g., treatment-emergent small-cell neuroendocrine prostate cancer (t-SCNC)), and Merkel cell carcinoma (MCC). In some embodiments, the cancer is neuroendocrine-positive (NE+) metastatic castration-resistant prostate cancer (mCRPC). In some embodiments, the cancer is small cell lung cancer (SCLC). In some embodiments, the cancer is NeuroD1+SCLC. In some embodiments,

In one aspect, the disclosure teaches the use of the kit of the disclosure for classifying sensitivity of the cancer in the subject to a Seneca Valley Virus (SVV).

### BRIEF DESCRIPTION OF THE DRAWINGS

FIG. 1 shows the result of ELN model run based on the ELN28 gene panel. Each triangle or circle represents a cell line with experimentally determined SVV sensitivity (triangles for SVV-sensitive cell lines and circles for SVV-resistant cell lines).

FIG. 2 shows the relationship between ELN signature score of the ELN28 gene panel and viral copy number for 14 PDX samples. The left chart plots the correlation between viral copy and the expression level of down-regulated genes, and the right chart plots the correlation between viral copy and the expression level of up-regulated genes. Correlation is calculated according to Spearman's correlation.

FIG. 3 shows the result of ELN model run based on the ELN28_reduced gene panel. Each triangle or circle represents a cell line with experimentally determined SVV sensitivity (triangles for SVV-sensitive cell lines and circles for SVV-resistant cell lines).

FIG. 4 shows the relationship between ELN signature score of the ELN28_reduced gene panel and viral copy number for 14 PDX samples. The left chart plots the correlation between viral copy and the expression level of down-regulated genes, and the right chart plots the correlation between viral copy and the expression level of up-regulated genes. Correlation is calculated according to Spearman's correlation.

FIG. 5 shows the ELN_1 gene signature scores and SVV-sensitivity prediction of various cell lines. Each point represents a CCEL, PDX or H1299 cell line, and the shape is based on experimentally determined SVV sensitivity. Squares represent SVV-sensitive cell lines that are lysed upon SVV infection. Triangles represent cell lines that can be chronically infected by SVV. Circles represent SVV-resistant cell lines.

FIG. 6 shows the ELN_3 gene signature scores and SVV-sensitivity prediction using CTC samples. Most CTC samples' sensitivity to platinum-based chemotherapy has been experimentally determined (circle: platinum-resistant; triangle: platinum-sensitive; star: unknown resistance to platinum-based chemotherapy).

FIG. 7 shows the ELN_3 gene signature scores and SVV-sensitivity prediction using CTC or tumor biopsy samples. Each triangle or circle represent a sample with experimentally determined sensitivity to platinum-based chemotherapy (circle: platinum-resistant; triangle: platinum-sensitive).

FIG. 8 shows the ELN_3 gene signature scores of CDX SCLC lines pre- and post-drug treatment. The lines connect the data points of each CDX line pre- and post-drug treatment.

FIG. 9 shows the SVV100 gene signature scores of various SCLC cell lines.

FIG. 10 shows the SVV100 gene signature score of various cell lines.

FIG. 11 shows the results of SVV viral replication in various PDX models upon SVV intratumoral administration.

FIG. 12A shows the results of SVV viral replication in tumors of mice treated as described in the legend (left) efficacy study in mice bearing LU5184 PDX model. FIG. 12B shows the results of SVV viral replication in tumors of mice treated as described in the legend (left) efficacy study in mice bearing LU5171 PDX model.

FIG. 13 shows SVV100 ELN gene signature scores and SVV-sensitivity prediction of various skin cancers using data from GSE39612.

FIG. 14 shows SVV100 ELN gene signature scores and SVV-sensitivity prediction of skin cancers using data from GSE22396.

FIG. 15 shows the results of in vitro SVV infectivity assay of multiple Merkel Cell Carcinoma (MCC) cell lines.

### DETAILED DESCRIPTION

### Overview

There is a need in the art for methods to determine whether a cancer will respond to treatment with a specific oncolytic virus. Seneca Valley Virus (SVV) is a promising oncolytic picornavirus for cancer therapies. However, one challenge for clinical development of SVV-based therapies is determining which groups of cancer patients would most likely benefit from SVV treatment.

The present disclosure is based, in part, on the discovery that the expression levels of certain genes are predictive of cancer cells' sensitivity to SVV infection. Such information may be used to predict the responsiveness of cancer patients to SVV treatment. Accordingly, in some embodiments, the present disclosure provides methods of evaluating the sensitivity of a cancer to SVV infection based on the expression level of one or more genes in the cancer. In some embodiments, the present disclosure provides methods of treating a cancer in a subject in need thereof, comprising administering a therapeutically effective amount of SVV or a polynucleotide encoding the SVV viral genome to the subject if the cancer is classified as sensitive to SVV infection.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term “approximately” or “about” refers to a range of values that fall within 10% in either direction (greater than or less than) of the stated reference value unless otherwise stated or otherwise evident from the context (except where such number would exceed 100% of a possible value).

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. Unless noted otherwise, the term “sequence identity” in the claims refers to sequence identity as calculated by Clustal Omega® version 1.2.4 using default parameters.

The terms “corresponding to” or “correspond to”, as used herein in relation to the amino acid or nucleic acid position(s), refer to the position(s) in a first polypeptide/polynucleotide sequence that aligns with a given amino acid/nucleic acid in a reference polypeptide/polynucleotide sequence when the first and the reference polypeptide/polynucleotide sequences are aligned. Alignment is performed by one of skill in the art using software designed for this purpose, for example, Clustal Omega version 1.2.4 with the default parameters for that version.

“Complementary” refers to the capacity for pairing, through base stacking and specific hydrogen bonding, between two sequences comprising naturally or non-naturally occurring (e.g., modified as described above) bases (nucleotides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject or contacting a composition with a cell and/or tissue.

“Treating” as used herein refers to delivering an agent or composition to a subject to affect a physiologic outcome. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and/or (c) curing the disease.

The term “effective amount” refers to the amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, number of cells/volume, particles/volume, (mass of the agent)/(mass of the subject), number of cells/(mass of subject), or particles/(mass of subject). The effective amount of a particular agent may also be expressed as the half-maximal effective concentration (ECso), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a virus, a polynucleotide (e.g., recombinant RNA molecule), or a particle-encapsulated polynucleotide described herein that is capable of being administered or delivered to a subject or cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The term “replication-competent viral genome” refers to a viral genome encoding all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable of transferring, encoding, or transporting another nucleic acid molecule.

As used herein, “plaque forming units” (PFU) refers to a measure of number of infectious virus particles. It is determined by plaque forming assay.

As used herein, “multiplicity of infection” (MOI) refers the average number of virus particles infecting each cell. MOI can be related to PFU by the following formula: Multiplicity of infection (MOI)=Plaque forming units (PFU) of virus used for infection/number of cells.

The term “SVV-sensitive” when used in reference to a cancer cell refers to a cancer cell (whether in vitro or in vivo) that is susceptible to infection with SVV. The SVV-sensitivity of a cancer cell can be determined by effective concentration 50 (EC50) in a cytotoxicity assay, wherein a lower EC50 is indicative of SVV-sensitivity.

The term “SVV-resistant” when used in reference to a cancer cell refers to a cancer cell (whether in vitro or in vivo) that is not susceptible to infection with SVV. The SVV-resistance of a cancer cell can be determined by effective concentration 50 (EC50) in a cytotoxicity assay, wherein a higher EC50 is indicative of SVV-resistance.

A biological marker or “biomarker” is a substance whose detection indicates a particular biological state, such as, for example, the sensitivity of a cancer to SVV infection. Biomarkers may be measured individually, or several biomarkers may be measured simultaneously. In some embodiments, the biomarker is the mRNA, cDNA, and/or protein product of a gene, or a portion thereof, expressed in a cancer cell, and the change in the expression level of the gene correlates with the SVV-sensitivity of the cancer cell. In some embodiments, the mRNA level is determined by the level of corresponding cDNA, or a fragment thereof, derived from the mRNA.

The terms “elevated”, “increased”, and “up-regulated” in reference to the expression level of a gene can be used interchangeably and mean that the expression level is higher than a reference expression level of the gene. The expression level may be mRNA expression level or protein expression level.

The terms “reduced”, “decreased”, and “down-regulated” in reference to the expression level of a gene can be used interchangeably and mean that the expression level is lower than a reference expression level of the gene. The expression level may be mRNA expression level or protein expression level.

A “reference gene expression level” or “reference expression level of a gene” used herein refers to the expression level of a particular gene in a reference sample (e.g., a control cell or a sample derived from a control subject population). In some embodiments, the reference gene expression level is obtained from a single source (e.g., a single patient or a single cell line). In some embodiments, the reference gene expression level is obtained from a population of different samples sharing a specific characteristic (e.g., sharing the characteristic of SVV sensitivity or resistance). In some embodiments, the reference gene expression level is obtained from the same sample or group of samples as the experimental gene expression level. In some embodiments, the reference gene expression level is the average gene expression level of a reference set of samples with known sensitivity to SVV infection (including SVV-sensitive and SVV-resistant samples). In some embodiments, the reference gene expression level is a pre-determined value. In some embodiments, the reference gene expression level is the expression level of a gene in a sample of non-cancerous cells (or multiple samples of non-cancerous cells). In some embodiments, the reference gene expression level is the average expression level of a gene in a group of cancer samples. In some embodiments, the reference gene expression level is the expression level of a gene in normal cells of the same origin in the same subject.

As used herein, an “expression profile” refers to the expression level for each gene in a collection of two or more genes. An expression profile may be derived from a subject prior to or subsequent to a diagnosis of cancer, from a biological sample collected from a subject at one or more time points prior to or following treatment or therapy, or from a healthy subject.

A “gene signature” or “gene panel” refers to a collection of genes. In some embodiments, the expression levels of the gene panel predict sensitivity of a cancer cell to SVV infection.

A “classifier” as used herein refers to a mathematical function that separates a collection of samples into two or more groups based on a particular metric or collection of metrics. In some embodiments, the classifier described herein is be used to separate SVV-sensitive cells and SVV-resistant cells into groups based on the metric of gene expression of a collection of genes.

A “sample” as used herein refers to a sample obtained from a biological subject, including a sample of biological tissue or fluid, obtained, reached, or collected in vivo or in situ. A sample may be from a region of a patient containing precancerous or cancer cells or tissues. Such samples can be, but are not limited to, organs, tissues, fractions, and cells isolated from a patient. Exemplary samples include but are not limited to a cell lysate, a cell culture, a cell line, a tissue, oral tissue, gastrointestinal tissue, an organ, an organelle, a biological fluid, a blood sample, a urine sample, a skin sample, and the like. Other exemplary samples include whole blood, partially purified blood, circulating tumor cells, PBMCs, tissue biopsies, and the like. In some embodiments, the sample is a tumor biopsy.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Methods of Evaluating the Sensitivity of a Cancer to Seneca Valley Virus (SVV) Infection

The present application is based, in part, on the finding that the expression levels of several groups of genes (e.g., those listed in Tables 2-14) in a cancer correlate with the cancer's sensitivity to Seneca Valley Virus (SVV) infection. Thus, in one aspect, provided herein are methods that use the expression level of one or more genes to evaluate the sensitivity of a cancer to SVV infection, and/or to classify the cancer as sensitive or resistant to SVV infection. A summary of some of the genes suitable for use according to the methods of the present disclosure is provided in Table 1 below.

TABLE 1
Summary of Genes Related to SVV Sensitivity

| | GenBank | GenBank Gene | | |
|---|---|---|---|---|
| Gene Symbol | GeneID | Aliase(s) | Gene Name | Uniprot Ref. |
| ANKRD20A8P | 729171 | ANKRD20B | ankyrin repeat domain 20 | Q5CZ79 |
| | | | family member A8, | |
| | | | pseudogene | |
| ASTN1 | 460 | ASTN | astrotactin 1 | O14525 |
| ATP2B2 | 491 | PMCA2, PMCA2a, | ATPase plasma | Q01814 |
| | | PMCA2i | membrane Ca2+ | |
| | | | transporting 2 | |
| BCRP2 | 400892 | BCR-2, BCR2, | BCR pseudogene 2 | |
| | | BCRL2 | | |
| CCDC157 | 550631 | | coiled-coil domain | Q569K6 |
| | | | containing 157 | |
| CCNJL | 79616 | | cyclin J like | Q8IV13 |
| CENPV | 201161 | 3110013H01Rik, | centromere protein V | Q7Z7K6 |
| | | CENP-V, PRR6, | | |
| | | p30 | | |
| CHRNA1 | 1134 | ACHRA, ACHRD, | cholinergic receptor | P02708 |
| | | CHRNA, CMS1A, | nicotinic alpha 1 subunit | |
| | | CMS1B, CMS2A, | | |
| | | FCCMS, SCCMS | | |
| CLDN5 | 7122 | AWAL, BEC1, | claudin 5 | O00501 |
| | | CPETRL1, | | |
| | | TMDVCF, TMVCF | | |
| CNRIP1 | 25927 | C2orf32, CRIP-1, | cannabinoid receptor | Q96F85 |
| | | CRIP1 | interacting protein 1 | |
| CPLX1 | 10815 | CPX-I, CPX1, | complexin 1 | O14810 |
| | | DEE63, EIEE63 | | |
| CYP7B1 | 9420 | CBAS3, CP7B, | cytochrome P450 family | O75881 |
| | | SPG5A | 7 subfamily B member 1 | |
| DACH1 | 1602 | DACH | dachshund family | Q9UI36 |
| | | | transcription factor 1 | |
| DCAF13 | 25879 | GM83, HSPC064, | DDB1 and CUL4 | Q9NV06 |
| | | Sof1, WDSOF1 | associated factor 13 | |
| DPP6 | 1804 | DPL1, DPPX, | dipeptidyl peptidase like | P42658 |
| | | MRD33, VF2 | 6 | |
| EXOSC3 | 51010 | CGI-102, PCH1B, | exosome component 3 | Q9NQT5 |
| | | RRP40, Rrp40p, | | |
| | | bA3J10.7, hRrp-40, | | |
| | | p10 | | |
| FAM118A | 55007 | C22orf8 | family with sequence | Q9NWS6 |
| | | | similarity 118 member A | |
| FAM155B | 27112 | CXorf63, | NALCN channel | O75949 |
| | | FAM155B, TED, | auxiliary factor 2 | |
| | | TMEM28, | | |
| | | bB57D9.1 | | |
| GID4 | 79018 | C17orf39, VID2, | GID complex subunit 4 | Q8IVV7 |
| | | VID24 | homolog | |
| GLCE | 26035 | HSEPI | glucuronic acid | O94923 |
| | | | epimerase | |
| GNAO1 | 2775 | DEE17, EIEE17, G- | G protein subunit alpha | P09471 |
| | | ALPHA-o, GNAO, | o1 | |
| | | HG1G, HLA-DQB1, | | |
| | | NEDIM | | |
| GRIP1 | 23426 | FRASRS3, GRIP | glutamate receptor | Q9Y3R0 |
| | | | interacting protein 1 | |
| HIP1 | 3092 | HIP-I, ILWEQ, | huntingtin interacting | O00291 |
| | | SHON, SHONbeta, | protein 1 | |
| | | SHONgamma | | |
| ITGA4 | 3676 | CD49D, IA4 | integrin subunit alpha 4 | P13612 |
| JPH1 | 56704 | CMT2K, JP-1, JP1 | junctophilin 1 | Q9HDC5 |
| KCNT2 | 343450 | DEE57, EIEE57, | potassium sodium- | Q6UVM3 |
| | | KCa4.2, SLICK, | activated channel | |
| | | SLO2.1 | subfamily T member 2 | |
| LAPTM5 | 7805 | CLAST6 | lysosomal protein | Q13571 |
| | | | transmembrane 5 | |
| LRFN5 | 145581 | C14orf146, | leucine rich repeat and | Q96NI6 |
| | | FIGLER8, SALM5 | fibronectin type III | |
| | | | domain containing 5 | |
| MAGEA10 | 4109 | CT1.10, MAGE10 | MAGE family member | P43363 |
| | | | A10 | |
| MCC | 4163 | MCC1 | MCC regulator of WNT | P23508 |
| | | | signaling pathway | |
| MIEF1 | 54471 | AltMIEF1, | mitochondrial elongation | Q9NQG6 |
| | | HSU79252, MID51- | factor 1 | |
| | | MP, SMCR7L, | | |
| | | dJ1104E15.3, | | |
| | | MIEF1 | | |
| MYT1L | 23040 | MRD39, NZF1, | myelin transcription | Q9UL68 |
| | | ZC2H2C2, | factor 1 like | |
| | | ZC2HC4B, myT1-L | | |
| NHLH2 | 4808 | HEN2, NSCL2, | nescient helix-loop-helix | Q02577 |
| | | bHLHa34 | 2 | |
| NSMF | 26012 | HH9, NELF | NMDA receptor | Q6X4W1 |
| | | | synaptonuclear signaling | |
| | | | and neuronal migration | |
| | | | factor | |
| NTNG2 | 84628 | LHLL9381, Lmnt2, | netrin G2 | Q96CW9 |
| | | NEDBASH, | | |
| | | NTNG1, | | |
| | | bA479K20.1 | | |
| PGPEP1L | 145814 | | pyroglutamyl-peptidase I | A6NFU8 |
| | | | like | |
| PMPCA | 23203 | Alpha-MPP, CLA1, | peptidase, mitochondrial | Q10713 |
| | | CPD3, INPP5E, | processing subunit alpha | |
| | | MAS2, P-55, | | |
| | | SCAR2 | | |
| PPFIA4 | 8497 | | PTPRF interacting | O75335 |
| | | | protein alpha 4 | |
| PPP1R17 | 10842 | C7orf16, GSBS | protein phosphatase 1 | O96001 |
| | | | regulatory subunit 17 | |
| PPP4R4 | 57718 | CFAP14, | protein phosphatase 4 | Q6NUP7 |
| | | KIAA1622, PP4R4 | regulatory subunit 4 | |
| PRDM8 | 56978 | EPM10, KMT8D, | PR/SET domain 8 | Q9NQV8 |
| | | PFM5 | | |
| PRKG2 | 5593 | PKG2, PRKGR2, | protein kinase cGMP- | Q13237 |
| | | cGK2, cGKII | dependent 2 | |
| RIPPLY2 | 134701 | C6orf159, SCDO6, | ripply transcriptional | Q5TAB7 |
| | | dJ237I15.1 | repressor 2 | |
| RNF112 | 7732 | BFP, ZNF179 | ring finger protein 112 | Q9ULX5 |
| RNF152P1 | 100419687 | | ring finger protein 152 | |
| | | | pseudogene 1 | |
| RPL23AP94 | 106481971 | | ribosomal protein L23a | |
| | | | pseudogene 94 | |
| RPL31P2 | 140753 | RPL31_25_1695, | ribosomal protein L31 | |
| | | dJ553F4.5 | pseudogene 2 | |
| RPS27P25 | 100271584 | RPS27_14_1288 | ribosomal protein S27 | |
| | | | pseudogene 25 | |
| RPS7P1 | 388363 | RPS7_4_1531 | ribosomal protein S7 | |
| | | | pseudogene 1 | |
| SCAMP1 | 9522 | SCAMP, SCAMP37 | secretory carrier | O15126 |
| | | | membrane protein 1 | |
| SCG3 | 29106 | SGIII | secretogranin III | Q8WXD2 |
| SELENOO | 83642 | SELO | selenoprotein O | Q9BVL4 |
| SOX5 | 6660 | L-SOX5, L-SOX5B, | SRY-box transcription | P35711 |
| | | L-SOX5F, | factor 5 | |
| | | LAMSHF | | |
| SYN2 | 6854 | SYNII | synapsin II | Q92777 |
| TAF1B | 9014 | MGC: 9349, RAF1B, | TATA-box binding | Q53T94 |
| | | RAFI63, SL1, | protein associated factor, | |
| | | TAFI63 | RNA polymerase I | |
| | | | subunit B | |
| TMEM249 | 340393 | C8ORFK29 | transmembrane protein | Q2WGJ8 |
| | | | 249 | |
| TRBVB | 28555 | TCRBV34S1 | T cell receptor beta | |
| | | | variable B (pseudogene) | |
| TTYH2 | 94015 | C17orf29 | tweety family member 2 | Q9BSA4 |
| UBR1 | 197131 | JBS | ubiquitin protein ligase | Q8IWV7 |
| | | | E3 component n-recognin 1 | |
| USB1 | 79650 | C16orf57, HVSL1, | U6 snRNA biogenesis | Q9BQ65 |
| | | Mpn1, PN, hUsb1 | phosphodiesterase 1 | |
| ACBD4 | 79777 | HMFT0700 | acyl-CoA binding domain | Q8NC06 |
| | | | containing 4 | |
| ADCY6 | 112 | AC6, LCCS8 | adenylate cyclase 6 | O43306 |
| AGRN | 375790 | AGRIN, CMS8, | agrin | O00468 |
| | | CMSPPD | | |
| ANO7L1 | 101927546 | ANO7P1, Clorf224, | anoctamin 7 like 1 | |
| | | TMEM16M | (pseudogene) | |
| ANXA1 | 301 | ANX1, LPC1 | annexin A1 | P04083 |
| APOL1 | 8542 | APO-L, APOL, | apolipoprotein L1 | O14791 |
| | | APOL-I, FSGS4 | | |
| ARHGEF16 | 27237 | GEF16, NBR | Rho guanine nucleotide | Q5VV41 |
| | | | exchange factor 16 | |
| ARHGEF34P | 728377 | | Rho guanine nucleotide | |
| | | | exchange factor 34, | |
| | | | pseudogene | |
| ARHGEF35 | 445328 | ARHGEF5L | Rho guanine nucleotide | A5YM69 |
| | | | exchange factor 35 | |
| ARID5A | 10865 | MRF-1, MRF1, | AT-rich interaction | Q03989 |
| | | RP11-363D14 | domain 5A | |
| B4GALT1 | 2683 | B4GAL-T1, | beta-1,4- | P15291 |
| | | CDG2D, GGTB2, | galactosyltransferase 1 | |
| | | GT1, GTB, | | |
| | | beta4Gal-T1 | | |
| C8orf89 | 100130301 | | chromosome 8 open | P0DMQ9 |
| | | | reading frame 89 | |
| CD226 | 10666 | DNAM-1, DNAM1, | CD226 molecule | Q15762 |
| | | PTA1, TLISA1 | | |
| CD9 | 928 | BTCC-1, DRAP-27, | CD9 molecule | P21926 |
| | | MIC3, MRP-1, | | |
| | | TSPAN-29, | | |
| | | TSPAN29 | | |
| CEP295 | 85459 | KIAA1731 | centrosomal protein 295 | Q9C0D2 |
| CLEC2D | 29121 | CLAX, LLT1, OCIL | C-type lectin domain | Q9UHP7 |
| | | | family 2 member D | |
| COPB1 | 1315 | BARMACS, COPB | COPI coat complex | P53618 |
| | | | subunit beta 1 | |
| CTAGE8 | 100142659 | CTAGE4 | CTAGE family member 8 | P0CG41 |
| CTSS | 1520 | | cathepsin S | P25774 |
| DENND2D | 79961 | | DENN domain | Q9H6A0 |
| | | | containing 2D | |
| DNAJC16 | 23341 | ERdj8 | DnaJ heat shock protein | Q9Y2G8 |
| | | | family (Hsp40) member | |
| | | | C16 | |
| EIF1P7 | 106481691 | | eukaryotic translation | |
| | | | initiation factor 1 | |
| | | | pseudogene 7 | |
| EPS8L2 | 64787 | DFNB106, EPS8R2 | EPS8 like 2 | Q9H6S3 |
| ERP27 | 121506 | C12orf46, PDIA8 | endoplasmic reticulum | Q96DN0 |
| | | | protein 27 | |
| ETV7 | 51513 | TEL-2, TEL2, | ETS variant transcription | Q9Y603 |
| | | TELB | factor 7 | |
| FAAP20 | 199990 | C1orf86, FP7162 | FA core complex | Q6NZ36 |
| | | | associated protein 20 | |
| FAM111A | 63901 | GCLEB, KCS2 | FAM111 trypsin like | Q96PZ2 |
| | | | peptidase A | |
| FAM183BP | 340286 | FAM183B, THEG6 | family with sequence | Q6ZVS7 |
| | | | similarity 183 member B, | |
| | | | pseudogene | |
| GRHL2 | 79977 | BOM, DFNA28, | grainyhead like | Q6ISB3 |
| | | ECTDS, PPCD4, | transcription factor 2 | |
| | | TFCP2L3 | | |
| GSDMD | 79792 | DF5L, DFNA5L, | gasdermin D | P57764 |
| | | FKSG10C1, | | |
| | | GSDMD | | |
| HLA-B | 3106 | AS, B-4901, HLAB | major histocompatibility | P01889 |
| | | | complex, class I, B | |
| HLA-C | 3107 | D6S204, HLA-JY3, | major histocompatibility | P10321 |
| | | HLAC, HLC-C, | complex, class I, C | |
| | | MHC, PSORS1 | | |
| HLA-E | 3133 | HLA-6.2, QA1 | major histocompatibility | P13747 |
| | | | complex, class I, E | |
| HLA-H | 3136 | HLAHP | major histocompatibility | P01893 |
| | | | complex, class I, H | |
| | | | (pseudogene) | |
| HOXC11 | 3227 | HOX3H | homeobox C11 | O43248 |
| IKBKE | 9641 | IKK-E, IKK-i, | inhibitor of nuclear factor | Q14164 |
| | | IKKE, IKKI | kappa B kinase subunit | |
| | | | epsilon | |
| IRS4 | 8471 | CHNG9, IRS-4, | insulin receptor substrate | O14654 |
| | | PY160 | 4 | |
| LTK | 4058 | TYK1 | leukocyte receptor | P29376 |
| | | | tyrosine kinase | |
| MAPK13 | 5603 | MAPK 13, MAPK- | mitogen-activated protein | O15264 |
| | | 13, PRKM13, | kinase 13 | |
| | | SAPK4, p38delta | | |
| MBOAT7 | 79143 | BB1, LENG4, | membrane bound O- | Q96N66 |
| | | LPIAT, LPLAT, | acyltransferase domain | |
| | | LRC4, MBOA7, | containing 7 | |
| | | MRT57, OACT7, | | |
| | | hMBOA-7 | | |
| MFAP1P1 | 646755 | | microfibril associated | |
| | | | protein 1 pseudogene 1 | |
| MICB | 4277 | PERB11.2 | MHC class I polypeptide- | Q29980 |
| | | | related sequence B | |
| MPIG6B | 80739 | C6orf25, G6b, G6b- | megakaryocyte and | O95866 |
| | | B, NG31, THAMY | platelet inhibitory | |
| | | | receptor G6b | |
| MUC17 | 140453 | MUC-17, MUC-3, | mucin 17, cell surface | Q685J3 |
| | | MUC3 | associated | |
| MUC6 | 4588 | MUC-6 | mucin 6, oligomeric | Q6W4X9 |
| | | | mucus/gel-forming | |
| MYL12A | 10627 | HEL-S-24, MLC- | myosin light chain 12A | P19105 |
| | | 2B, MLCB, | | |
| | | MRCL3, MRLC3, | | |
| | | MYL2B | | |
| MYO5B | 4645 | DIAR2, MVID1 | myosin VB | Q9ULV0 |
| NBPF14 | 25832 | DJ328E19.C1.1, | NBPF member 14 | Q5TI25 |
| | | NBPF | | |
| NLRP14 | 338323 | CLR11.2, GC-LRR, | NLR family pyrin | Q86W24 |
| | | NALP14, NOD5, | domain containing 14 | |
| | | PAN8 | | |
| NPC2 | 10577 | EDDM1, HE1 | NPC intracellular | P61916 |
| | | | cholesterol transporter 2 | |
| NUCB2 | 4925 | HEL-S-109, NEFA | nucleobindin 2 | P80303 |
| PARP9 | 83666 | ARTD9, BAL, | poly(ADP-ribose) | Q8IXQ6 |
| | | BAL1, MGC: 7868 | polymerase family | |
| | | | member 9 | |
| PDCL3P4 | 285359 | | PDCL3 pseudogene 4 | |
| PLCG2 | 5336 | APLAID, FCAS3, | phospholipase C gamma | P16885 |
| | | PLC-IV, PLC- | 2 | |
| | | gamma-2 | | |
| PLPP2 | 8612 | LPP2, PAP-2c, | phospholipid phosphatase | O43688 |
| | | PAP2-g, PPAP2C | 2 | |
| PROM2 | 150696 | PROML2 | prominin 2 | Q8N271 |
| PRSS22 | 64063 | BSSP-4, SP001LA, | serine protease 22 | Q9GZN4 |
| | | hBSSP-4 | | |
| PSMB8 | 5696 | ALDD, D6S216, | proteasome 20S subunit | P28062 |
| | | D6S216E, JMP, | beta 8 | |
| | | LMP7, NKJO, | | |
| | | PRAAS1, PSMB5i, | | |
| | | RING10 | | |
| PSMB9 | 5698 | LMP2, PRAAS3, | proteasome 20S subunit | P28065 |
| | | PSMB6i, RING12, | beta 9 | |
| | | beta1i | | |
| RAPGEF3 | 10411 | CAMP-GEFI, | Rap guanine nucleotide | O95398 |
| | | EPAC, EPAC1, | exchange factor 3 | |
| | | HSU79275, bcm910 | | |
| RHBDF1 | 64285 | C16orf8, Dist1, | rhomboid 5 homolog 1 | Q96CC6 |
| | | EGFR-RS, gene-89, | | |
| | | gene-90, hDist1 | | |
| SHISAL2A | 348378 | FAM159A, | shisa like 2A | Q6UWV7 |
| | | PRO7171, | | |
| | | WWLS2783 | | |
| SLC52A1 | 55065 | GPCR42, GPR172B, | solute carrier family 52 | Q9NWF4 |
| | | PAR2, RBFVD, | member 1 | |
| | | RFT1, RFVT1, | | |
| | | hRFT1, huPAR-2 | | |
| SMAD7 | 4092 | CRCS3, MADH7, | SMAD family member 7 | O15105 |
| | | MADH8 | | |
| SNED1 | 25992 | IRE-BP1, SST3, | sushi, nidogen and EGF | Q8TER0 |
| | | Snep | like domains 1 | |
| STAT1 | 6772 | CANDF7, IMD31A, | signal transducer and | P42224 |
| | | IMD31B, IMD31C, | activator of transcription | |
| | | ISGF-3, STAT91 | 1 | |
| STAT6 | 6778 | D12S1644, IL-4- | signal transducer and | P42226 |
| | | STATB, STAT6C, | activator of transcription | |
| | | STAT6 | 6 | |
| SYTL2 | 54843 | CHR11SYT, EXO4, | synaptotagmin like 2 | Q9HCH5 |
| | | PPP1R151, | | |
| | | SGA72M, SLP2, | | |
| | | SLP2A | | |
| TAP1 | 6890 | ABC17, ABCB2, | transporter 1, ATP | Q03518 |
| | | APT1, D6S114E, | binding cassette | |
| | | PSF-1, PSF1, | subfamily B member | |
| | | RING4*0102N, | | |
| | | TAP1N, TAP1 | | |
| TEX54 | 111216277 | | testis expressed 54 | A0A1B0GVG6 |
| TMCO4 | 255104 | | transmembrane and | Q5TGY1 |
| | | | coiled-coil domains 4 | |
| TMED11P | 100379220 | p24a1, p24alpha1 | transmembrane p24 | |
| | | | trafficking protein 11, | |
| | | | pseudogene | |
| TMEM80 | 283232 | | transmembrane protein 80 | Q96HE8 |
| TMEM9B | 56674 | C11orf15 | TMEM9 domain family | Q9NQ34 |
| | | | member B | |
| TNFAIP3 | 7128 | A20, AISBL, | TNF alpha induced | P21580 |
| | | OTUD7C, | protein 3 | |
| | | TNFA1P2 | | |
| TNFRSF10B | 8795 | CD262, DR5, | TNF receptor superfamily | O14763 |
| | | KILLER, | member 10b | |
| | | KILLER/DR5, | | |
| | | TRAIL-R2, | | |
| | | TRAILR2, TRICK2, | | |
| | | TRICK2A, | | |
| | | TRICK2B, | | |
| | | TRICKB, ZTNFR9 | | |
| TNFRSF14 | 8764 | ATAR, CD270, | TNF receptor superfamily | Q92956 |
| | | HVEA, HVEM, | member 14 | |
| | | LIGHTR, TR2 | | |
| TYR | 7299 | ATN, CMM8, | tyrosinase | P14679 |
| | | OCA1, OCA1A, | | |
| | | OCAIA, SHEP3 | | |
| UNC93B1 | 81622 | IIAE1, UNC93, | unc-93 homolog B1, TLR | Q9H1C4 |
| | | UNC93B, Unc-93B1 | signaling regulator | |
| USP43 | 124739 | | ubiquitin specific | Q70EL4 |
| | | | peptidase 43 | |
| VWA1 | 64856 | HMNMYO, WARP | von Willebrand factor A | Q6PCB0 |
| | | | domain containing 1 | |
| VWA5A | 4013 | BCSC-1, BCSC1, | von Willebrand factor A | O00534 |
| | | LOH11CR2A | domain containing 5A | |
| YAP1P1 | 442266 | | YAP1 pseudogene 1 | |
| CFAP44 | 55779 | SPGF20, WDR52 | cilia and flagella | Q96MT7 |
| | | | associated protein 44 | |
| C6orf62 | 81688 | Nbla00237, XTP12, | chromosome 6 open | Q9GZU0 |
| | | dJ30M3.2 | reading frame 62 | |
| EDEM1 | 9695 | EDEM | ER degradation | Q92611 |
| | | | enhancing alpha- | |
| | | | mannosidase like protein 1 | |
| PCYOX1L | 78991 | | prenylcysteine oxidase 1 | Q8NBM8 |
| | | | like | |
| ANKH | 56172 | ANK, CCAL2, | ANKH inorganic | Q9HCJ1 |
| | | CMDJ, CPPDD, | pyrophosphate transport | |
| | | HANK, MANK, | regulator | |
| | | SLC62A1 | | |
| ORC5 | 5001 | ORC5L, ORC5P, | origin recognition | O43913 |
| | | ORC5T, PPP1R117 | complex subunit 5 | |
| KIRREL2 | 84063 | FILTRIN, NEPH3, | kirre like nephrin family | Q6UWL6 |
| | | NLG1 | adhesion molecule 2 | |
| NME1-NME2 | 654364 | NM23-LV, NMELV | NME1-NME2 | Q32Q12 |
| | | | readthrough | |
| YBX2 | 51087 | CONTRIN, CSDA3, | Y-box binding protein 2 | Q9Y2T7 |
| | | DBPC, MSY2 | | |

In some embodiments, the present disclosure provides methods of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer. In some embodiments, expression levels of the one or more genes are used to predict the sensitivity of the cancer to SVV infection.

Whether a given sample or cell line is sensitive to SVV can be determined by methods known in the art. For example, cytotoxicity assays may be used to determine the effective concentration (EC50) value of the cells to SVV according to Reddy et al., J Natl Cancer Inst. 2007 Nov. 7; 99(21):1623-33, the content of which is incorporated by reference in its entirety. Therein, an EC50 value of less than 10 indicated that the corresponding cells were sensitive to SVV infection, whereas an EC50 values of greater than 10000 indicated that the corresponding cells were resistant to SVV. Certain samples or cells may have “moderate sensitivity” to SVV infection. Although such cancer samples or cells are still sensitive to SVV, a higher dose of SVV may be required to achieve high infection rate. In some embodiments, samples or cells with moderate sensitivity to SVV have EC50 values that are between those of SVV-sensitive cells and those of SVV-resistant cells. In some embodiments, SVV infection in samples or cells with moderate sensitivity results in prolonged or chronic infection rather than cell lysis.

In some embodiments, the gene panels described herein for determining the sensitivity of a cancer to SVV infection may be derived as follows: a training set of cancer samples is obtained including both SVV-sensitive and SVV-resistant samples. The gene expression profile of each cancer sample is determined by RNA-seq and used in an elastic net (ELN) search to identify genes with predictive power for classifying samples into SVV-sensitive (S) or SVV-resistant (R). An exemplary ELN search method is described in Zhou and Hastie, Journal of the Royal Statistical Society, vol B 67, pg 301, 2005, the content of which is incorporated by reference in its entirety. In some embodiments, the signature genes identified in the ELN search can be ranked in the gene panel based on their frequency of occurrence in the ELN search, which can be calculated by the number of runs in which the gene is selected in the ELN search divided by the total number of runs of the ELN search.

In some embodiments, the gene panels described herein for determining the sensitivity of a cancer to SVV infection may be derived as follows: a training set of cancer samples are obtained, which includes both SVV-sensitive and SVV-resistant samples/cells. The gene expression profile of each cancer sample/cell is determined by RNA-seq. A differential expression analysis based on the gene expression profiles to obtain signature genes that are differentially expressed between the resistant and sensitive samples while accounting for the overall variations between the samples (e.g., between cell line samples and PDX samples).

In some embodiments, a cancer sample may be classified as SVV-sensitive or SVV-resistant by comparing expression level(s) of the one or more genes of the disclosure (e.g., those in a gene panel) against those of a reference sample set comprising both SVV-sensitive and SVV-resistant samples. In some embodiments, the expression profile of the one or more genes of the samples may be subject to a Gene Set Variation Analysis (GSVA) run which can differentiate SVV-sensitive samples from SVV-resistant samples, and which in turn classifies the cancer sample as SVV-sensitive or SVV-resistant. An exemplary GSVA run is illustrated in Example 1 of the application based on Hanzelmann et al., BMC Bioinformatics. 2013 Jan. 16; 14:7, the content of which is incorporated herein by reference in its entirety.

In some embodiments, a cancer sample may be classified as SVV-sensitive or SVV-resistant by 1) transforming the expression level(s) of the one or more genes of the disclosure (e.g., those in a gene panel) into a sample “score” based on a transformation matrix; and 2) comparing the sample score to a reference score. If the sample score is higher compared to the reference score, the corresponding cancer sample is determined to be sensitive to SVV infection. If the sample score is lower compared to the reference score, the corresponding cancer sample is determined to be resistant to SVV infection. In some embodiments, the transformation matrix and the reference score may be derived from a reference set of samples with known sensitivity to SVV infection (including SVV-sensitive and SVV-resistant samples). In some embodiments, GVSA may be used to derive the transformation matrix and the reference score.

In some embodiments, a cancer sample with a sample score that is at least 2-fold, 3-fold, 4-fold, 5-fold, 6-fold, 7-fold, 8-fold, 9-fold, or 10-fold, higher than a reference score is classified as SVV-sensitive. In some embodiments, a cancer sample with a sample score that is at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200%, higher that a reference score is classified as SVV-sensitive. In some embodiments, a cancer sample with a sample score that is at least 2-fold, 3-fold, 4-fold, 5-fold, 6-fold, 7-fold, 8-fold, 9-fold, or 10-fold, lower than a reference score is classified as SVV-resistant. In some embodiments, a cancer sample with a sample score that is at least 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200%, lower that a reference score is classified as SVV-resistant.

In some embodiments, the present disclosure provides methods of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer. In some embodiments, expression levels of the one or more genes are used to predict the sensitivity of the cancer to SVV. In some embodiments, a population of cancer subjects that have received SVV treatment are divided into two groups based on cancer's sensitivity/responsiveness to SVV treatment i.e., a sensitive group and a resistant group. The expression levels of the one or more genes provided herein for each cancer are analyzed, and the results can be provided to a classifier to obtain score(s). Reference scores can be generated based on the scores of SVV-sensitive cancers and the scores of SVV resistant cancers. In some embodiments, such reference scores can be used to predict a cancer's sensitivity to SVV based on the expression level of the one or more genes. In some embodiments, the method comprises determining the probability of the cancer being sensitive to SVV infection by comparing the score(s) of the sample to reference score(s).

In some embodiments, the present disclosure provides methods of treating a cancer in a subject in need thereof, comprising: (a) determining the expression level of one or more genes in the cancer; (b) classifying the cancer as sensitive to Seneca Valley Virus (SVV) infection based on the expression level of the one or more genes as determined in (a); and (c) administering a therapeutically effective amount of a Seneca Valley Virus (SVV) or a polynucleotide encoding the SVV viral genome to the subject if the cancer is classified as sensitive to SVV infection in step (b).

In some embodiments, the present disclosure provides methods of evaluating the sensitivity of a cancer to Seneca Valley Virus (SVV) infection, comprising determining the expression level of one or more genes in the cancer and classifying the cancer as sensitive or resistant to SVV infection based on the expression level of the one or more genes. In some embodiments, the method comprises administering a SVV or a polynucleotide encoding the SVV viral genome to the cancer.

In some embodiments, the present disclosure provides methods of selecting a subject suffering from a cancer for treatment with a Seneca Valley Virus (SVV) or a polynucleotide encoding the SVV viral genome, comprising: (a) determining the expression level of one or more genes in the cancer; (b) classifying the cancer as sensitive to SVV infection based on the expression level of the one or more genes as determined in (a); and (c) selecting the subject for treatment with the SVV or the polynucleotide encoding the SVV viral genome if the cancer is classified as sensitive to SVV infection in (b). In some embodiments, the method comprises administering the SVV or the polynucleotide encoding the SVV viral genome to the selected subject.

Tables 2-14 provide various groups of genes (e.g., gene panels) that can be used to determine the sensitivity of a cancer to SVV infection. In some embodiments, the one or more genes comprise any one of the genes listed in Tables 2-14 or a combination thereof. In some embodiments, the one or more genes do not comprise ANTXR1 (NCBI Gene ID: 84168; Uniprot Ref: Q9H6X2). In some embodiments, the one or more genes do not comprise IFI35 (GenBank Gene ID: 3430; Uniprot Ref: P80217).

In some embodiments, the one or more genes comprise at least one gene selected from at least one of Tables 1-14. In some embodiments, the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 genes selected from at least one of Tables 1-14. In some embodiments, the one or more genes have a frequency of at least 5% in at least one of Tables 2-11. In some embodiments, the one or more genes have a frequency of at least 10%, 15%, 20%, 25%, 30%, 35%, 40%, 45%, 50%, 55%, 60%, 65%, 70%, 75%, or 80% in at least one of Tables 2-11. In some embodiments, the one or more genes do not comprise ANTXR1. In some embodiments, the one or more genes do not comprise IFI35.

The frequency of the genes as noted in Table 2 or 3 refers to the number of runs in which the indicated gene is selected in an elastic net modeling divided by the total number of runs of the elastic net modeling, as shown in the Example section of the present disclosure. Similarly, the genes in Tables 4-7 are ordered according to their frequency in the elastic net modeling, with the upregulated/downregulated gene having the highest frequency listed at the top of each table.

TABLE 2
ELN28 Gene Panel

| Upregulated Genes | | Downregulated genes | |
|---|---|---|---|
| Gene symbol | Frequency (%) | Gene Symbol | Frequency (%) |
| RPL23AP94 | 45.6 | TAP1 | 67.6 |
| SYN2 | 36.0 | PLCG2 | 50.9 |
| NSMF | 30.9 | ARHGEF35 | 45.8 |
| PPFIA4 | 28.3 | ARHGEF16 | 42.7 |
| SELENOO | 26.5 | MICB | 38.6 |
| CHRNA1 | 23.1 | TMCO4 | 36.0 |
| GNAO1 | 20.9 | RAPGEF3 | 33.5 |
| TMEM249 | 19.3 | NPC2 | 30.5 |
| SCG3 | 17.6 | MYL12A | 26.1 |
| CCNJL | 16.3 | ANO7L1 | 25.9 |
| JPH1 | 14.7 | TNFRSF10B | 23.6 |
| LRFN5 | 12.8 | HLA-B | 20.6 |
| CPLX1 | 12.5 | PROM2 | 19.0 |
| SCAMP1 | 12.0 | USP43 | 18.4 |
| ASTN1 | 9.7 | RHBDF1 | 17.0 |
| TRBVB | 9.4 | HLA-C | 15.9 |
| KCNT2 | 8.6 | SYTL2 | 15.9 |
| ATP2B2 | 8.1 | ETV7 | 15.7 |
| CYP7B1 | 7.6 | DENND2D | 15.1 |
| PPP1R17 | 6.9 | HOXC11 | 13.8 |
| CENPV | 6.4 | CLEC2D | 13.6 |
| CCDC157 | 6.0 | ARHGEF34P | 13.5 |
| SOX5 | 5.7 | TNFRSF14 | 12.8 |
| BCRP2 | 5.6 | CD226 | 12.5 |
| FAM118A | 5.6 | NBPF14 | 11.9 |
| TTYH2 | 5.4 | PSMB9 | 11.4 |
| ANKRD20A8P | 5.2 | CD9 | 9.9 |
| CLDN5 | 5.2 | ACBD4 | 8.6 |
| NHLH2 | 5.2 | HLA-E | 8.3 |
| MYT1L | 5.1 | TNFAIP3 | 7.5 |
| | | GRHL2 | 7.2 |
| | | ANXA1 | 7.1 |
| | | CTAGE8 | 7.0 |
| | | IRS4 | 6.9 |
| | | TMED11P | 6.3 |
| | | MPIG6B | 5.7 |
| | | VWA5A | 5.6 |
| | | ERP27 | 5.3 |
| | | PRSS22 | 5.3 |
| | | CTSS | 5.2 |
| | | YBX2 | 5.2 |
| | | STAT6 | 5.1 |
| | | PLPP2 | 5.0 |

TABLE 3
ELN28_reduced Gene Panel

| Upregulated Genes | | Downregulated genes | |
|---|---|---|---|
| Gene symbol | Frequency (%) | Gene Symbol | Frequency (%) |
| RPL23AP94 | 45.6 | TAP1 | 67.6 |
| SYN2 | 36.0 | PLCG2 | 50.9 |
| NSMF | 30.9 | ARHGEF35 | 45.8 |
| PPFIA4 | 28.3 | ARHGEF16 | 42.7 |
| SELENOO | 26.5 | MICB | 38.6 |
| CHRNA1 | 23.1 | TMCO4 | 36.0 |
| GNAO1 | 20.9 | RAPGEF3 | 33.5 |
| TMEM249 | 19.3 | NPC2 | 30.5 |
| SCG3 | 17.6 | MYL12A | 26.1 |
| CCNJL | 16.3 | ANO7L1 | 25.9 |
| JPH1 | 14.7 | TNFRSF10B | 23.6 |
| LRFN5 | 12.8 | HLA-B | 20.6 |
| CPLX1 | 12.5 | PROM2 | 19.0 |
| SCAMP1 | 12.0 | USP43 | 18.4 |
| ASTN1 | 9.7 | RHBDF1 | 17.0 |
| | | HLA-C | 15.9 |
| | | SYTL2 | 15.9 |
| | | ETV7 | 15.7 |
| | | DENND2D | 15.1 |
| | | HOXC11 | 13.8 |

TABLE 4
ELN_1 Gene Panel

| Upregulated Genes | Downregulated genes |
|---|---|
| PRDM8 | HLA-C |
| CNRIP1 | PARP9 |
| MCC | DENND2D |
| JPH1 | ETV7 |
| TAF1B | HLA-H |
| GLCE | TMEM9B |
| MIEF1 | STAT6 |
| DACH1 | PDCL3P4 |
| USB1 | STAT1 |
| TTYH2 | NUCB2 |
| ITGA4 | COPB1 |
| PPP4R4 | SMAD7 |
| RIPPLY2 | CEP295 |
| RNF112 | FAM111A |
| EXOSC3 | UNC93B1 |
| NTNG2 | TMCO4 |
| PMPCA | MBOAT7 |
| HIP1 | PSMB8 |
| RPS7P1 | B4GALT1 |
| GID4 | IKBKE |
| UBR1 | EPS8L2 |
| DCAF13 | TMEM80 |
| | MYO5B |
| | DNAJC16 |
| | GSDMD |
| | MAPK13 |

TABLE 5
ELN_C Gene Panel

| Upregulated Genes | Downregulated genes |
|---|---|
| RPS27P25 | ARID5A |
| GRIP1 | AGRN |
| DPP6 | FAAP20 |
| CCDC157 | VWA1 |
| PRDM8 | HLA-C |
| CNRIP1 | PARP9 |
| MCC | DENND2D |
| JPH1 | ETV7 |
| TAF1B | HLA-H |
| GLCE | TMEM9B |
| MIEF1 | STAT6 |
| DACH1 | PDCL3P4 |
| USB1 | STAT1 |
| TTYH2 | NUCB2 |
| ITGA4 | COPB1 |
| PPP4R4 | SMAD7 |
| RIPPLY2 | CEP295 |
| RNF112 | FAM111A |
| EXOSC3 | UNC93B1 |
| NTNG2 | TMCO4 |
| PMPCA | MBOAT7 |
| HIP1 | PSMB8 |
| RPS7P1 | B4GALT1 |
| GID4 | IKBKE |
| UBR1 | EPS8L2 |
| DCAF13 | TMEM80 |
| | MYO5B |
| | DNAJC16 |
| | GSDMD |
| | MAPK13 |

TABLE 6
ELN_2 Gene Panel

| Upregulated Genes | Downregulated genes |
|---|---|
| CNRIP1 | HLA-C |
| RNF152P1 | FAM183BP |
| GLCE | NLRP14 |
| FAM155B | TEX54 |
| PGPEP1L | SHISAL2A |
| RPL31P2 | YAP1P1 |
| PRKG2 | APOL1 |
| MAGEA10 | SNED1 |
| LAPTM5 | ADCY6 |
| DCAF13 | MUC17 |
| | MFAP1P1 |
| | LTK |
| | TYR |
| | SLC52A1 |
| | HOXC11 |
| | MUC6 |
| | C8orf89 |
| | EIF1P7 |
| | SMAD7 |

TABLE 7
ELN_3 Gene Panel

| Upregulated Genes | Downregulated genes |
|---|---|
| CNRIP1 | SHISAL2A |
| FAM155B | HLA-C |
| DCAF13 | ADCY6 |
| JPH1 | GSDMD |
| PCYOX1L | SMAD7 |
| NSMF | CFAP44 |
| ANKH | C6orf62 |
| ORC5 | TNFRSF14 |
| GLCE | EDEM1 |
| KIRREL2 | |
| NME1-NME2 | |

TABLE 8
Up-regulated Genes in the SVV100 Panel
Up-regulated Genes in the SVV100 Panel

ABCG2
ACSM3
AFAP1L2
AIFM2
ANXA3
APOBEC3B
APOBEC3D
APOL1
AREG
ARHGEF16
ASB9
BHLHE40
C19orf33
C1RL
CAPG
CASP10
CCDC69
CELSR1
CTSZ
CYP2S1
DMKN
DRAM1
DSG2
EREG
ETV4
FAM83H
FAM83H-AS1
FBXO27
FIBCD1
FRK
GDF15
GGT1
GJB2
HCP5
HLA-B
HLA-C
HLA-F
HNF1B
ICOSLG
IFI35
IKBKE
IL15RA
IL18
IL6R
IRF5
ISG20
KDELR3
KYNU
LIPH
LRRC8E
LTBR
MCTP2
MICA
MICB
MLKL
MOCOS
NQO1
PDGFB
PHLDA2
PLD1
PNPLA4
PROSER2
PRRG4
PSD4
PYGL
RAB20
RBM47
RHOD
RNF207
SAT1
SDSL
SERPINA1
SGMS2
SH2D3A
SH3TC1
SLC16A5
SLC52A3
SP100
SQRDL
STAT6
STXBP2
TAP1
TCIRG1
TFAP2C
TGFA
TINAGL1
TMBIM1
TMCO4
TMEM144
TMEM92
TNFRSF10A
TNFRSF14
TNFSF10
TNS3
TPD52L1
TRIM47
USP43
UTRN
VAMP8
VDR

TABLE 9
Down-regulated Genes in the SVV100 Panel
Down-regulated Genes in the SVV100 Panel

| | | | |
|---|---|---|---|
| ADAM23 | FLRT2 | NELL2 | SCN3A |
| ARL10 | FOXO6 | NFASC | SEMA6D |
| ATP1A3 | FSD1 | NFATC4 | SLC4A8 |
| ATP8A1 | GABRA3 | NRN1 | SMAD9 |
| BCL11A | GNAO1 | NTM | SNAP25 |
| BEX1 | GNB3 | NYNRIN | SOX11 |
| BEX4 | GPC2 | PDZD4 | SPTBN4 |
| BRSK1 | GPR162 | PDZRN3 | SULT4A1 |
| BSN | GPR173 | PHF21B | SYP |
| CACNA2D1 | IGFBPL1 | PHYHIPL | SYT11 |
| CADPS | ISL1 | PKIA | SYT14 |
| CAND2 | JAKMIP2 | POU3F2 | TCF4 |
| CHGA | JAM3 | PPM1E | TENM4 |
| CHRNB2 | KIAA1549L | PRDM8 | THY1 |
| CNRIP1 | KIF5A | RAB39A | TMEFF1 |
| CPT1C | KIF5C | RCOR2 | TMSB15A |
| DACH1 | LIN28B | RIMS3 | TRO |
| DCHS1 | LRCH2 | RNF150 | TSPYL5 |
| DOK6 | LZTS1 | ROBO2 | UNC13A |
| DPYSL3 | MAP1A | RTN1 | UNC5B |
| EBF1 | MEIS1 | RUNX1T1 | UNC80 |
| EFNB3 | MEX3A | SAMD11 | WDR17 |
| ELOVL4 | NAP1L3 | SATB1 | ZNF354C |
| FAM155A | NCAM1 | SCG3 | ZNF521 |
| FAT3 | NCAM1-AS1 | SCN2A | ZNF804A |

TABLE 10
Up-regulated Genes in the SVV-SCLC Panel
Upregulated Gene in the SVV-SCLC Panel

| | | | |
|---|---|---|---|
| HCN1 | RNF112 | SLC9B2 | DOT1L |
| PPP1R17 | TTC24 | WASF1 | ELP1 |
| GAP43 | PSTPIP1 | JPH1 | FLAD1 |
| DCX | MAPT | CDIP1 | DNAJC19 |
| NHLH2 | PROK2 | NAT14 | SNAPC4 |
| SCG3 | SYP | NSMF | STAT5B |
| RIMS4 | DISP3 | AGAP3 | SUZ12P1 |
| GDAP1L1 | RCOR2 | PDXP | EXOSC2 |
| CNRIP1 | SYN2 | RRP1B | NUBP2 |
| RTN1 | CFAP61 | MIR5010 | KRBA1 |
| CELF3 | RPL23AP94 | SKP2 | XPO5 |
| ELAVL4 | ATP6V1G2 | FAM120C | FBRSL1 |
| MFAP4 | TRMT61A | PSKH1 | ERAL1 |
| CHRNA1 | PRKG2 | FARSA | C1orf109 |
| TAGLN3 | FOXO6 | ABCC5 | HNRNPA1P4 |
| CAMKV | FOXO3B | BCL7A | LCMT2 |
| ZDHHC22 | DISP2 | ACTR3B | ENTR1 |
| ASPDH | RPS6KL1 | WDCP | ECSIT |
| PLPP7 | SCAMP5 | PRR3 | ZNF786 |
| ASTN1 | A1BG | ZNF286A | SMARCD1 |
| DACH1 | TMEM198 | UCK2 | ABHD10 |
| NKAIN1 | GSTM5P1 | ODC1 | MAZ |
| MYT1L | NUS1P1 | ATP6V0E2 | DPH5 |
| ESPNL | SHF | NCKAP5L | KAT14 |
| RHBDL3 | MEX3A | SNRPE | EXOSC3 |
| DUSP26 | TMEM249 | DNAAF2 | LIG3 |
| PHF21B | DOC2A | CD320 | UBE2G2 |
| GRIK5 | PPFIA4 | GTPBP3 | GEMIN2 |
| NOS2 | DUX4L37 | SGTA | POGLUT1 |
| SEMA6D | ZBTB18 | PMPCA | TMEM199 |
| EYA2 | ANKRD13B | ARMC6 | C17orf75 |
| RIIAD1 | CENPV | HEATR3 | BORCS8 |
| NKD1 | KATNB1 | CCT7 | EMC4 |
| CRB1 | STXBP1 | APEX1 | NKIRAS2 |

TABLE 11
Down-regulated Genes in the SVV-SCLC Panel
Downregulated Gene in the SVV-SCLC Panel

| | | | |
|---|---|---|---|
| CTSS | SYTL2 | NFKBIZ | POLD4 |
| ANXA1 | SP110 | ANXA4 | AIFM2 |
| CRYBG1 | SPINK1 | PRSS23 | NPC2 |
| ARHGEF35 | TNFRSF14 | SAT1 | CCDC50 |
| HLA-C | PSMB8 | SMAGP | H6PD |
| VWA5A | NPAS2 | PLEKHG6 | TRIM56 |
| ADGRG6 | MICA | MOCOS | PATE2 |
| CTAGE8 | PLEKHN1 | NOTCH2NLA | TMEM87B |
| HOXC11 | OSR1 | ISG20 | HLA-G |
| APOBEC3B | ARHGEF16 | CEACAM1 | HSBP1L1 |
| CXCL8 | HLA-E | RAPGEF3 | RIPK2 |
| CTAGE4 | GIPC2 | CASP8 | RIPK1 |
| HLA-B | ATP2C2 | ZNF860 | MPIG6B |
| SLFN5 | RHBDF1 | OR10AD1 | MAN2B2 |
| NBPF14 | PRR15 | CLEC2D | MYL12A |
| CLDN1 | HFE | GCNT4 | MARCKSL1P1 |
| USP43 | TMCO4 | OCLN | NBPF10 |
| PSMB9 | HES1 | SSH3 | ACBD4 |
| CD9 | OVOL1 | APOBEC3F | LRRK1 |
| LAMP3 | FAM111A | FCHSD1 | TWF1 |
| LRAT | IKBKE | IRF1 | PATE1 |
| TNFRSF10B | PLEK2 | NCEH1 | NPHP3 |
| PLCG2 | UNC93B1 | PSD4 | CMTM6 |
| ARHGEF5 | DENND2D | CDRT4 | BIRC2 |
| TMEM144 | VDR | SAMD4A | GNS |
| IGF2BP2 | GSDMD | BIRC3 | LINC00672 |
| TAP1 | SP100 | ELF1 | LRRFIP1 |
| TNFRSF10A | CCDC68 | RELB | CCDC186 |
| GPR39 | PGGHG | ANO7L1 | ZBTB4 |
| CDCP1 | NLRP14 | CFAP52 | MYL12B |
| HLA-H | HLA-F | GCNT2 | UHMK1 |
| MALL | PRRG2 | ETV7 | TMBIM6 |
| ITGB6 | GBP3 | ERP27 | SLAIN2 |
| MST1R | HLA-K | ADAP2 | POC1B |
| CASP4 | STAT6 | MAP3K6 | FRYL |
| ACPP | IER3 | WBP1L | UEVLD |
| IFIH1 | SH2D4A | PLD1 | CLPTM1 |
| PTGER4 | GRHL2 | PHF11 | SCAF11 |
| TNFRSF10C | PARP9 | RNASEL | KRT18P47 |
| PSORS1C1 | TNFAIP3 | RPL11P3 | PHACTR4 |
| OR2A7 | IL13RA1 | MMP13 | WASHC4 |
| MICB | IFI35 | | |

In some embodiments, the one or more genes comprise all genes in one of Tables 2-7, or all genes in Tables 8-9, or all genes in Tables 10-11.

In some embodiments, the one or more genes comprise all genes in Table 2 (RPL23AP94, SYN2, NSMF, PPFIA4, SELENOO, CHRNA1, GNAO1, TMEM249, SCG3, CCNJL, JPH1, LRFN5, CPLX1, SCAMPI, ASTN1, TRBVB, KCNT2, ATP2B2, CYP7B1, PPP1R17, CENPV, CCDC157, SOX5, BCRP2, FAM118A, TTYH2, ANKRD20A8P, CLDN5, NHLH2, MYT1L, TAP1, PLCG2, ARHGEF35, ARHGEF16, MICB, TMCO4, RAPGEF3, NPC2, MYL12A, ANO7L1, TNFRSF10B, HLA-B, PROM2, USP43, RHBDF1, HLA-C, SYTL2, ETV7, DENND2D, HOXC11, CLEC2D, ARHGEF34P, TNFRSF14, CD226, NBPF14, PSMB9, CD9, ACBD4, HLA-E, TNFAIP3, GRHL2, ANXA1, CTAGE8, IRS4, TMED11P, MPIG6B, VWA5A, ERP27, PRSS22, CTSS, YBX2, STAT6, and PLPP2).

In some embodiments, the one or more genes comprise all genes in Table 3 (RPL23AP94, SYN2, NSMF, PPFIA4, SELENOO, CHRNA1, GNAO1, TMEM249, SCG3, CCNJL, JPH1, LRFN5, CPLX1, SCAMPI, ASTN1, TAP1, PLCG2, ARHGEF35, ARHGEF16, MICB, TMCO4, RAPGEF3, NPC2, MYL12A, ANO7L1, TNFRSF10B, HLA-B, PROM2, USP43, RHBDF1, HLA-C, SYTL2, ETV7, DENND2D, and HOXC11).

In some embodiments, the one or more genes comprise all genes in Table 4.

In some embodiments, the one or more genes comprise all genes in Table 5.

In some embodiments, the one or more genes comprise all genes in Table 6.

In some embodiments, the one or more genes comprise all genes in Table 7.

In some embodiments, the one or more genes comprise all genes in Tables 8-9.

In some embodiments, the one or more genes comprise all genes in Tables 10-11.

In some embodiments, the one or more genes comprise at least one gene encoding a protein with adaptive immunity and/or immune response function.

In some embodiments, the one or more genes comprise at least one gene encoding a protein with adaptive immunity function selected from TAP1, MICB, HLA-B, HLA-C, HLA-E, HLA-H, and CTSS, as shown in Table 12 below. In some embodiments, the one or more genes comprise at least 2, 3, 4, 5, 6, or 7 genes selected from the group consisting of TAP1, MICB, HLA-B, HLA-C, HLA-E, HLA-H, and CTSS.

In some embodiments, the one or more genes comprise at least one gene encoding a protein with immune response function selected from CLEC2D, TNFRSF14, CD226, TNFAIP3, ANXA1, GSDMD, IKBKE, MAPK13, PARP9, TMEM9B, and UNC93B1, as shown in Table 12 below. In some embodiments, the one or more genes comprise at least 2, 3, 4, 5, 6, 7, 8, 9, 10, or 11 genes selected from the group consisting of CLEC2D, TNFRSF14, CD226, TNFAIP3, ANXA1, GSDMD, IKBKE, MAPK13, PARP9, TMEM9B, and UNC93B1.

In some embodiments, the one or more genes comprise at least one gene encoding a protein with cell adhesion/migration function selected from PPFIA4, LRFN5, ASTN1, MYL12A, CLDN5, CD9, SNED1, ITGA4, B4GALT1, and VWA1, as shown in Table 12 below. In some embodiments, the one or more genes comprise at least 2, 3, 4, 5, 6, 7, 8, 9, or 10 genes selected from the group consisting of PPFIA4, LRFN5, ASTN1, MYL12A, CLDN5, CD9, SNED1, ITGA4, B4GALT1, and VWA1.

In some embodiments, the one or more genes comprise at least one gene encoding a protein with cellular RNA processing function selected from YBX2, EXOSC3, TAF1B, and USB1, as shown in Table 12 below. In some embodiments, the one or more genes comprise at least 2, 3, or 4 genes selected from the group consisting of YBX2, EXOSC3, TAF1B, and USB1.

In some embodiments, the one or more genes comprise at least one gene encoding a protein with intracellular transportation function selected from SCG3, CPLX1, SCAMPI, SYTL2, CTAGE8, SLC52A1, HIP1, COPB1, and MYO5B, as shown in Table 12 below. In some embodiments, the one or more genes comprise at least 2, 3, 4, 5, 6, 7, 8, or 9 genes selected from the group consisting of SCG3, CPLX1, SCAMPI, SYTL2, CTAGE8, SLC52A1, HIP1, COPB1, and MYO5B.

In some embodiments, the one or more genes comprise at least one gene encoding a protein with DNA repair function selected from TYR, FAAP20, and FAM111A, as shown in Table 12 below. In some embodiments, the one or more genes comprise at least 2, or 3 genes selected from the group consisting of TYR, FAAP20, and FAM111A.

In some embodiments, the one or more genes comprise at least one gene encoding a protein with G-protein signaling function selected from GNAO1, PLCG2, ARHGEF35, ARHGEF16, RAPGEF3, DENND2D, and ADCY6, as shown in Table 12 below. In some embodiments, the one or more genes comprise at least 2, 3, 4, 5, 6, or 7 genes selected from the group consisting of GNAO1, PLCG2, ARHGEF35, ARHGEF16, RAPGEF3, DENND2D, and ADCY6.

In some embodiments, the one or more genes comprise at least one gene encoding a protein with neurotransmission function selected from SYN2, NSMF, CHRNA1, KCNT2, ATP2B2, TTYH2, CNRIP1, FAM155B, NTNG2, and AGRN, as shown in Table 12 below. In some embodiments, the one or more genes comprise at least 2, 3, 4, 5, 6, 7, 8, 9 or 10 genes selected from the group consisting of SYN2, NSMF, CHRNA1, KCNT2, ATP2B2, TTYH2, CNRIP1, FAM155B, NTNG2, and AGRN.

In some embodiments, the one or more genes comprise at least one gene encoding a transcription factor selected from ETV7, HOXC11, SOX5, NHLH2, MYT1L, GRHL2, STAT6, DACH1, and ARID5A, as shown in Table 12 below. In some embodiments, the one or more genes comprise at least 2, 3, 4, 5, 6, 7, 8, or 9 genes selected from the group consisting of ETV7, HOXC11, SOX5, NHLH2, MYT1L, GRHL2, STAT6, DACH1, and ARID5A.

In some embodiments, the one or more genes comprise at least one gene encoding a protein with ubiquitination function selected from GID4, RNF112, and UBR1, as shown in Table 12 below. In some embodiments, the one or more genes comprise at least 2 or 3 genes selected from the group consisting of GID4, RNF112, and UBR1.

TABLE 12
Summary of Exemplary Gene function

| Gene Symbol | Gene Function | Gene Symbol | Gene Function |
|---|---|---|---|
| TAP1 | Adaptive immunity | CLEC2D | Immune response |
| MICB | Adaptive immunity | TNFRSF14 | Immune response |
| HLA-B | Adaptive immunity | CD226 | Immune response |
| HLA-C | Adaptive immunity | TNFAIP3 | Immune response |
| HLA-E | Adaptive immunity | ANXA1 | Immune response |
| CTSS | Adaptive immunity | GSDMD | Immune response |
| HLA-H | Adaptive immunity | IKBKE | Immune response |
| PPFIA4 | Cell adhesion/migration | MAPK13 | Immune response |
| LRFN5 | Cell adhesion/migration | PARP9 | Immune response |
| ASTN1 | Cell adhesion/migration | TMEM9B | Immune response |
| MYL12A | Cell adhesion/migration | UNC93B1 | immune response |
| CLDN5 | Cell adhesion/migration | SYN2 | Neurotransmission |
| CD9 | Cell adhesion/migration | NSMF | Neurotransmission |
| SNED1 | Cell adhesion/migration | CHRNA1 | Neurotransmission |
| ITGA4 | Cell adhesion/migration | KCNT2 | Neurotransmission |
| B4GALT1 | Cell adhesion/migration | ATP2B2 | Neurotransmission |
| VWA1 | Cell adhesion/migration | TTYH2 | Neurotransmission |
| YBX2 | Cellular RNA processing | CNRIP1 | Neurotransmission |
| EXOSC3 | Cellular RNA processing | FAM155B | Neurotransmission |
| TAF1B | Cellular RNA processing | NTNG2 | Neurotransmission |
| USB1 | Cellular RNA processing | AGRN | Neurotransmission |
| SCG3 | Intracellular transportation | ETV7 | Transcription factor |
| CPLX1 | Intracellular transportation | HOXC11 | Transcription factor |
| SCAMP1 | Intracellular transportation | SOX5 | Transcription factor |
| SYTL2 | Intracellular transportation | NHLH2 | Transcription factor |
| CTAGE8 | Intracellular transportation | MYT1L | Transcription factor |
| SLC52A1 | Intracellular transportation | GRHL2 | Transcription factor |
| HIP1 | Intracellular transportation | STAT1 | Transcription factor |
| COPB1 | Intracellular transportation | STAT6 | Transcription factor |
| MYO5B | Intracellular transportation | DACH1 | Transcription factor |
| TYR | DNA repair | ARID5A | Transcription factor |
| FAAP20 | DNA repair | GID4 | Ubiquitination |
| FAM111A | DNA repair | RNF112 | Ubiquitination |
| GNAO1 | G protein signaling | UBR1 | Ubiquitination |
| PLCG2 | G protein signaling | | |
| ARHGEF35 | G protein signaling | | |
| ARHGEF16 | G protein signaling | | |
| RAPGEF3 | G protein signaling | | |
| DENND2D | G protein signaling | | |
| ADCY6 | G protein signaling | | |

### Exemplary Gene Combinations

In some embodiments, the present disclosure provides methods of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer. In some embodiments, expression levels of the one or more genes are used to predict the sensitivity of the cancer to SVV infection.

In some embodiments, the one or more genes comprise TAP1. In some embodiments, the one or more genes comprise PLCG2. In some embodiments, the one or more genes comprise both TAP1 and PLCG2. Both genes have a frequency of more than 50% in Tables 2-3. In some embodiments, the one or more genes do not comprise ANTXR1. In some embodiments, the one or more genes do not comprise IFI35.

In some embodiments, the one or more genes comprise at least one gene selected from the group consisting of TAP1, PLCG2, ARHGEF35, RPL23AP94, and ARHGEF16. Each of these genes have a frequency of at least 40% in Tables 2-3. In some embodiments, the one or more genes comprise at least 2, 3, 4, or 5 genes selected from the group consisting of TAP1, PLCG2, ARHGEF35, RPL23AP94, and ARHGEF16. In some embodiments, the one or more genes do not comprise ANTXR1. In some embodiments, the one or more genes do not comprise IFI35.

In some embodiments, the one or more genes comprise at least one gene selected from the group consisting of TAP1, PLCG2, ARHGEF35, RPL23AP94, ARHGEF16, MICB, SYN2, TMCO4, RAPGEF3, NSMF, and NPC2. Each of these genes have a frequency of at least 30% in Tables 2-3. In some embodiments, the one or more genes comprise at least 2, 3, 4, 5, 6, 7, 8, 9, 10, or 11 genes selected from the group consisting of TAP1, PLCG2, ARHGEF35, RPL23AP94, ARHGEF16, MICB, SYN2, TMCO4, RAPGEF3, NSMF, NPC2. In some embodiments, the one or more genes do not comprise ANTXR1. In some embodiments, the one or more genes do not comprise IFI35.

In some embodiments, the one or more genes comprise at least one gene selected from the group consisting of TAP1, PLCG2, ARHGEF35, RPL23AP94, ARHGEF16, MICB, SYN2, TMCO4, RAPGEF3, NSMF, NPC2, PPFIA4, SELENOO, MYL12A, ANO7L1, TNFRSF10B, CHRNA1, GNAO1, and HLA-B. Each of these genes have a frequency of at least 20% in Tables 2-3. In some embodiments, the one or more genes comprise at least 2, 3, 4, 5, 6, 7, 8, 9, 10, 11, 12, 13, 14, 15, 16, 17, 18 or 19 genes selected from the group consisting of TAP1, PLCG2, ARHGEF35, RPL23AP94, ARHGEF16, MICB, SYN2, TMCO4, RAPGEF3, NSMF, NPC2, PPFIA4, SELENOO, MYL12A, ANO7L1, TNFRSF10B, CHRNA1, GNAO1, and HLA-B. In some embodiments, the one or more genes do not comprise ANTXR1. In some embodiments, the one or more genes do not comprise IFI35.

In some embodiments, the one or more genes comprise at least one gene selected from the group consisting of TAP1, PLCG2, ARHGEF35, RPL23AP94, ARHGEF16, MICB, SYN2, TMCO4, RAPGEF3, NSMF, NPC2, PPFIA4, SELENOO, MYL12A, ANO7L1, TNFRSF10B, CHRNA1, GNAO1, HLA-B, TMEM249, PROM2, USP43, SCG3, RHBDF1, CCNJL, HLA-C, SYTL2, ETV7, and DENND2D. Each of these genes have a frequency of at least 15% in Tables 2-3. In some embodiments, the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, or 29 genes selected from the group consisting of TAP1, PLCG2, ARHGEF35, RPL23AP94, ARHGEF16, MICB, SYN2, TMCO4, RAPGEF3, NSMF, NPC2, PPFIA4, SELENOO, MYL12A, ANO7L1, TNFRSF10B, CHRNA1, GNAO1, HLA-B, TMEM249, PROM2, USP43, SCG3, RHBDF1, CCNJL, HLA-C, SYTL2, ETV7, and DENND2D. In some embodiments, the one or more genes do not comprise ANTXR1. In some embodiments, the one or more genes do not comprise IFI35.

In some embodiments, the one or more genes comprise one of the gene combinations selected from Table 13 below. In some embodiments, the one or more genes comprise two genes selected from combinations #1 to #55 of Table 13 below. In some embodiments, the one or more genes comprise three genes selected from combinations #56 to #220 of Table 13 below. In some embodiments, the one or more genes comprise four genes selected from combinations #221 to #550 of Table 13 below. In some embodiments, the one or more genes comprise five genes selected from combinations #551 to #1012 of Table 13 below. In some embodiments, the one or more genes do not comprise ANTXR1. In some embodiments, the one or more genes do not comprise IFI35.

TABLE 13
Non-limiting Examples of Gene Combination (C#)

| C# | Genes |
|---|---|
| 1 | RPL23AP94, SYN2 |
| 2 | RPL23AP94, NSMF |
| 3 | RPL23AP94, TAP1 |
| 4 | RPL23AP94, PLCG2 |
| 5 | RPL23AP94, ARHGEF35 |
| 6 | RPL23AP94, ARHGEF16 |
| 7 | RPL23AP94, MICB |
| 8 | RPL23AP94, TMCO4 |
| 9 | RPL23AP94, RAPGEF3 |
| 10 | RPL23AP94, NPC2 |
| 11 | SYN2, NSMF |
| 12 | SYN2, TAP1 |
| 13 | SYN2, PLCG2 |
| 14 | SYN2, ARHGEF35 |
| 15 | SYN2, ARHGEF16 |
| 16 | SYN2, MICB |
| 17 | SYN2, TMCO4 |
| 18 | SYN2, RAPGEF3 |
| 19 | SYN2, NPC2 |
| 20 | NSMF, TAP1 |
| 21 | NSMF, PLCG2 |
| 22 | NSMF, ARHGEF35 |
| 23 | NSMF, ARHGEF16 |
| 24 | NSMF, MICB |
| 25 | NSMF, TMCO4 |
| 26 | NSMF, RAPGEF3 |
| 27 | NSMF, NPC2 |
| 28 | TAP1, PLCG2 |
| 29 | TAP1, ARHGEF35 |
| 30 | TAP1, ARHGEF16 |
| 31 | TAP1, MICB |
| 32 | TAP1, TMCO4 |
| 33 | TAP1, RAPGEF3 |
| 34 | TAP1, NPC2 |
| 35 | PLCG2, ARHGEF35 |
| 36 | PLCG2, ARHGEF16 |
| 37 | PLCG2, MICB |
| 38 | PLCG2, TMCO4 |
| 39 | PLCG2, RAPGEF3 |
| 40 | PLCG2, NPC2 |
| 41 | ARHGEF35, ARHGEF16 |
| 42 | ARHGEF35, MICB |
| 43 | ARHGEF35, TMCO4 |
| 44 | ARHGEF35, RAPGEF3 |
| 45 | ARHGEF35, NPC2 |
| 46 | ARHGEF16, MICB |
| 47 | ARHGEF16, TMCO4 |
| 48 | ARHGEF16, RAPGEF3 |
| 49 | ARHGEF16, NPC2 |
| 50 | MICB, TMCO4 |
| 51 | MICB, RAPGEF3 |
| 52 | MICB, NPC2 |
| 53 | TMCO4, RAPGEF3 |
| 54 | TMCO4, NPC2 |
| 55 | RAPGEF3, NPC2 |
| 56 | RPL23AP94, SYN2, NSMF |
| 57 | RPL23AP94, SYN2, TAP1 |
| 58 | RPL23AP94, SYN2, PLCG2 |
| 59 | RPL23AP94, SYN2, ARHGEF35 |
| 60 | RPL23AP94, SYN2, ARHGEF16 |
| 61 | RPL23AP94, SYN2, MICB |
| 62 | RPL23AP94, SYN2, TMCO4 |
| 63 | RPL23AP94, SYN2, RAPGEF3 |
| 64 | RPL23AP94, SYN2, NPC2 |
| 65 | RPL23AP94, NSMF, TAP1 |
| 66 | RPL23AP94, NSMF, PLCG2 |
| 67 | RPL23AP94, NSMF, ARHGEF35 |
| 68 | RPL23AP94, NSMF, ARHGEF16 |
| 69 | RPL23AP94, NSMF, MICB |
| 70 | RPL23AP94, NSMF, TMCO4 |
| 71 | RPL23AP94, NSMF, RAPGEF3 |
| 72 | RPL23AP94, NSMF, NPC2 |
| 73 | RPL23AP94, TAP1, PLCG2 |
| 74 | RPL23AP94, TAP1, ARHGEF35 |
| 75 | RPL23AP94, TAP1, ARHGEF16 |
| 76 | RPL23AP94, TAP1, MICB |
| 77 | RPL23AP94, TAP1, TMCO4 |
| 78 | RPL23AP94, TAP1, RAPGEF3 |
| 79 | RPL23AP94, TAP1, NPC2 |
| 80 | RPL23AP94, PLCG2, ARHGEF35 |
| 81 | RPL23AP94, PLCG2, ARHGEF16 |
| 82 | RPL23AP94, PLCG2, MICB |
| 83 | RPL23AP94, PLCG2, TMCO4 |
| 84 | RPL23AP94, PLCG2, RAPGEF3 |
| 85 | RPL23AP94, PLCG2, NPC2 |
| 86 | RPL23AP94, ARHGEF35, ARHGEF16 |
| 87 | RPL23AP94, ARHGEF35, MICB |
| 88 | RPL23AP94, ARHGEF35, TMCO4 |
| 89 | RPL23AP94, ARHGEF35, RAPGEF3 |
| 90 | RPL23AP94, ARHGEF35, NPC2 |
| 91 | RPL23AP94, ARHGEF16, MICB |
| 92 | RPL23AP94, ARHGEF16, TMCO4 |
| 93 | RPL23AP94, ARHGEF16, RAPGEF3 |
| 94 | RPL23AP94, ARHGEF16, NPC2 |
| 95 | RPL23AP94, MICB, TMCO4 |
| 96 | RPL23AP94, MICB, RAPGEF3 |
| 97 | RPL23AP94, MICB, NPC2 |
| 98 | RPL23AP94, TMCO4, RAPGEF3 |
| 99 | RPL23AP94, TMCO4, NPC2 |
| 100 | RPL23AP94, RAPGEF3, NPC2 |
| 101 | SYN2, NSMF, TAP1 |
| 102 | SYN2, NSMF, PLCG2 |
| 103 | SYN2, NSMF, ARHGEF35 |
| 104 | SYN2, NSMF, ARHGEF16 |
| 105 | SYN2, NSMF, MICB |
| 106 | SYN2, NSMF, TMCO4 |
| 107 | SYN2, NSMF, RAPGEF3 |
| 108 | SYN2, NSMF, NPC2 |
| 109 | SYN2, TAP1, PLCG2 |
| 110 | SYN2, TAP1, ARHGEF35 |
| 111 | SYN2, TAP1, ARHGEF16 |
| 112 | SYN2, TAP1, MICB |
| 113 | SYN2, TAP1, TMCO4 |
| 114 | SYN2, TAP1, RAPGEF3 |
| 115 | SYN2, TAP1, NPC2 |
| 116 | SYN2, PLCG2, ARHGEF35 |
| 117 | SYN2, PLCG2, ARHGEF16 |
| 118 | SYN2, PLCG2, MICB |
| 119 | SYN2, PLCG2, TMCO4 |
| 120 | SYN2, PLCG2, RAPGEF3 |
| 121 | SYN2, PLCG2, NPC2 |
| 122 | SYN2, ARHGEF35, ARHGEF16 |
| 123 | SYN2, ARHGEF35, MICB |
| 124 | SYN2, ARHGEF35, TMCO4 |
| 125 | SYN2, ARHGEF35, RAPGEF3 |
| 126 | SYN2, ARHGEF35, NPC2 |
| 127 | SYN2, ARHGEF16, MICB |
| 128 | SYN2, ARHGEF16, TMCO4 |
| 129 | SYN2, ARHGEF16, RAPGEF3 |
| 130 | SYN2, ARHGEF16, NPC2 |
| 131 | SYN2, MICB, TMCO4 |
| 132 | SYN2, MICB, RAPGEF3 |
| 133 | SYN2, MICB, NPC2 |
| 134 | SYN2, TMCO4, RAPGEF3 |
| 135 | SYN2, TMCO4, NPC2 |
| 136 | SYN2, RAPGEF3, NPC2 |
| 137 | NSMF, TAP1, PLCG2 |
| 138 | NSMF, TAP1, ARHGEF35 |
| 139 | NSMF, TAP1, ARHGEF16 |
| 140 | NSMF, TAP1, MICB |
| 141 | NSMF, TAP1, TMCO4 |
| 142 | NSMF, TAP1, RAPGEF3 |
| 143 | NSMF, TAP1, NPC2 |
| 144 | NSMF, PLCG2, ARHGEF35 |
| 145 | NSMF, PLCG2, ARHGEF16 |
| 146 | NSMF, PLCG2, MICB |
| 147 | NSMF, PLCG2, TMCO4 |
| 148 | NSMF, PLCG2, RAPGEF3 |
| 149 | NSMF, PLCG2, NPC2 |
| 150 | NSMF, ARHGEF35, ARHGEF16 |
| 151 | NSMF, ARHGEF35, MICB |
| 152 | NSMF, ARHGEF35, TMCO4 |
| 153 | NSMF, ARHGEF35, RAPGEF3 |
| 154 | NSMF, ARHGEF35, NPC2 |
| 155 | NSMF, ARHGEF16, MICB |
| 156 | NSMF, ARHGEF16, TMCO4 |
| 157 | NSMF, ARHGEF16, RAPGEF3 |
| 158 | NSMF, ARHGEF16, NPC2 |
| 159 | NSMF, MICB, TMCO4 |
| 160 | NSMF, MICB, RAPGEF3 |
| 161 | NSMF, MICB, NPC2 |
| 162 | NSMF, TMCO4, RAPGEF3 |
| 163 | NSMF, TMCO4, NPC2 |
| 164 | NSMF, RAPGEF3, NPC2 |
| 165 | TAP1, PLCG2, ARHGEF35 |
| 166 | TAP1, PLCG2, ARHGEF16 |
| 167 | TAP1, PLCG2, MICB |
| 168 | TAP1, PLCG2, TMCO4 |
| 169 | TAP1, PLCG2, RAPGEF3 |
| 170 | TAP1, PLCG2, NPC2 |
| 171 | TAP1, ARHGEF35, ARHGEF16 |
| 172 | TAP1, ARHGEF35, MICB |
| 173 | TAP1, ARHGEF35, TMCO4 |
| 174 | TAP1, ARHGEF35, RAPGEF3 |
| 175 | TAP1, ARHGEF35, NPC2 |
| 176 | TAP1, ARHGEF16, MICB |
| 177 | TAP1, ARHGEF16, TMCO4 |
| 178 | TAP1, ARHGEF16, RAPGEF3 |
| 179 | TAP1, ARHGEF16, NPC2 |
| 180 | TAP1, MICB, TMCO4 |
| 181 | TAP1, MICB, RAPGEF3 |
| 182 | TAP1, MICB, NPC2 |
| 183 | TAP1, TMCO4, RAPGEF3 |
| 184 | TAP1, TMCO4, NPC2 |
| 185 | TAP1, RAPGEF3, NPC2 |
| 186 | PLCG2, ARHGEF35, ARHGEF16 |
| 187 | PLCG2, ARHGEF35, MICB |
| 188 | PLCG2, ARHGEF35, TMCO4 |
| 189 | PLCG2, ARHGEF35, RAPGEF3 |
| 190 | PLCG2, ARHGEF35, NPC2 |
| 191 | PLCG2, ARHGEF16, MICB |
| 192 | PLCG2, ARHGEF16, TMCO4 |
| 193 | PLCG2, ARHGEF16, RAPGEF3 |
| 194 | PLCG2, ARHGEF16, NPC2 |
| 195 | PLCG2, MICB, TMCO4 |
| 196 | PLCG2, MICB, RAPGEF3 |
| 197 | PLCG2, MICB, NPC2 |
| 198 | PLCG2, TMCO4, RAPGEF3 |
| 199 | PLCG2, TMCO4, NPC2 |
| 200 | PLCG2, RAPGEF3, NPC2 |
| 201 | ARHGEF35, ARHGEF16, MICB |
| 202 | ARHGEF35, ARHGEF16, TMCO4 |
| 203 | ARHGEF35, ARHGEF16, RAPGEF3 |
| 204 | ARHGEF35, ARHGEF16, NPC2 |
| 205 | ARHGEF35, MICB, TMCO4 |
| 206 | ARHGEF35, MICB, RAPGEF3 |
| 207 | ARHGEF35, MICB, NPC2 |
| 208 | ARHGEF35, TMCO4, RAPGEF3 |
| 209 | ARHGEF35, TMCO4, NPC2 |
| 210 | ARHGEF35, RAPGEF3, NPC2 |
| 211 | ARHGEF16, MICB, TMCO4 |
| 212 | ARHGEF16, MICB, RAPGEF3 |
| 213 | ARHGEF16, MICB, NPC2 |
| 214 | ARHGEF16, TMCO4, RAPGEF3 |
| 215 | ARHGEF16, TMCO4, NPC2 |
| 216 | ARHGEF16, RAPGEF3, NPC2 |
| 217 | MICB, TMCO4, RAPGEF3 |
| 218 | MICB, TMCO4, NPC2 |
| 219 | MICB, RAPGEF3, NPC2 |
| 220 | TMCO4, RAPGEF3, NPC2 |
| 221 | RPL23AP94, SYN2, NSMF, TAP1 |
| 222 | RPL23AP94, SYN2, NSMF, PLCG2 |
| 223 | RPL23AP94, SYN2, NSMF, ARHGEF35 |
| 224 | RPL23AP94, SYN2, NSMF, ARHGEF16 |
| 225 | RPL23AP94, SYN2, NSMF, MICB |
| 226 | RPL23AP94, SYN2, NSMF, TMCO4 |
| 227 | RPL23AP94, SYN2, NSMF, RAPGEF3 |
| 228 | RPL23AP94, SYN2, NSMF, NPC2 |
| 229 | RPL23AP94, SYN2, TAP1, PLCG2 |
| 230 | RPL23AP94, SYN2, TAP1, ARHGEF35 |
| 231 | RPL23AP94, SYN2, TAP1, ARHGEF16 |
| 232 | RPL23AP94, SYN2, TAP1, MICB |
| 233 | RPL23AP94, SYN2, TAP1, TMCO4 |
| 234 | RPL23AP94, SYN2, TAP1, RAPGEF3 |
| 235 | RPL23AP94, SYN2, TAP1, NPC2 |
| 236 | RPL23AP94, SYN2, PLCG2, ARHGEF35 |
| 237 | RPL23AP94, SYN2, PLCG2, ARHGEF16 |
| 238 | RPL23AP94, SYN2, PLCG2, MICB |
| 239 | RPL23AP94, SYN2, PLCG2, TMCO4 |
| 240 | RPL23AP94, SYN2, PLCG2, RAPGEF3 |
| 241 | RPL23AP94, SYN2, PLCG2, NPC2 |
| 242 | RPL23AP94, SYN2, ARHGEF35, ARHGEF16 |
| 243 | RPL23AP94, SYN2, ARHGEF35, MICB |
| 244 | RPL23AP94, SYN2, ARHGEF35, TMCO4 |
| 245 | RPL23AP94, SYN2, ARHGEF35, RAPGEF3 |
| 246 | RPL23AP94, SYN2, ARHGEF35, NPC2 |
| 247 | RPL23AP94, SYN2, ARHGEF16, MICB |
| 248 | RPL23AP94, SYN2, ARHGEF16, TMCO4 |
| 249 | RPL23AP94, SYN2, ARHGEF16, RAPGEF3 |
| 250 | RPL23AP94, SYN2, ARHGEF16, NPC2 |
| 251 | RPL23AP94, SYN2, MICB, TMCO4 |
| 252 | RPL23AP94, SYN2, MICB, RAPGEF3 |
| 253 | RPL23AP94, SYN2, MICB, NPC2 |
| 254 | RPL23AP94, SYN2, TMCO4, RAPGEF3 |
| 255 | RPL23AP94, SYN2, TMCO4, NPC2 |
| 256 | RPL23AP94, SYN2, RAPGEF3, NPC2 |
| 257 | RPL23AP94, NSMF, TAP1, PLCG2 |
| 258 | RPL23AP94, NSMF, TAP1, ARHGEF35 |
| 259 | RPL23AP94, NSMF, TAP1, ARHGEF16 |
| 260 | RPL23AP94, NSMF, TAP1, MICB |
| 261 | RPL23AP94, NSMF, TAP1, TMCO4 |
| 262 | RPL23AP94, NSMF, TAP1, RAPGEF3 |
| 263 | RPL23AP94, NSMF, TAP1, NPC2 |
| 264 | RPL23AP94, NSMF, PLCG2, ARHGEF35 |
| 265 | RPL23AP94, NSMF, PLCG2, ARHGEF16 |
| 266 | RPL23AP94, NSMF, PLCG2, MICB |
| 267 | RPL23AP94, NSMF, PLCG2, TMCO4 |
| 268 | RPL23AP94, NSMF, PLCG2, RAPGEF3 |
| 269 | RPL23AP94, NSMF, PLCG2, NPC2 |
| 270 | RPL23AP94, NSMF, ARHGEF35, ARHGEF16 |
| 271 | RPL23AP94, NSMF, ARHGEF35, MICB |
| 272 | RPL23AP94, NSMF, ARHGEF35, TMCO4 |
| 273 | RPL23AP94, NSMF, ARHGEF35, RAPGEF3 |
| 274 | RPL23AP94, NSMF, ARHGEF35, NPC2 |
| 275 | RPL23AP94, NSMF, ARHGEF16, MICB |
| 276 | RPL23AP94, NSMF, ARHGEF16, TMCO4 |
| 277 | RPL23AP94, NSMF, ARHGEF16, RAPGEF3 |
| 278 | RPL23AP94, NSMF, ARHGEF16, NPC2 |
| 279 | RPL23AP94, NSMF, MICB, TMCO4 |
| 280 | RPL23AP94, NSMF, MICB, RAPGEF3 |
| 281 | RPL23AP94, NSMF, MICB, NPC2 |
| 282 | RPL23AP94, NSMF, TMCO4, RAPGEF3 |
| 283 | RPL23AP94, NSMF, TMCO4, NPC2 |
| 284 | RPL23AP94, NSMF, RAPGEF3, NPC2 |
| 285 | RPL23AP94, TAP1, PLCG2, ARHGEF35 |
| 286 | RPL23AP94, TAP1, PLCG2, ARHGEF16 |
| 287 | RPL23AP94, TAP1, PLCG2, MICB |
| 288 | RPL23AP94, TAP1, PLCG2, TMCO4 |
| 289 | RPL23AP94, TAP1, PLCG2, RAPGEF3 |
| 290 | RPL23AP94, TAP1, PLCG2, NPC2 |
| 291 | RPL23AP94, TAP1, ARHGEF35, ARHGEF16 |
| 292 | RPL23AP94, TAP1, ARHGEF35, MICB |
| 293 | RPL23AP94, TAP1, ARHGEF35, TMCO4 |
| 294 | RPL23AP94, TAP1, ARHGEF35, RAPGEF3 |
| 295 | RPL23AP94, TAP1, ARHGEF35, NPC2 |
| 296 | RPL23AP94, TAP1, ARHGEF16, MICB |
| 297 | RPL23AP94, TAP1, ARHGEF16, TMCO4 |
| 298 | RPL23AP94, TAP1, ARHGEF16, RAPGEF3 |
| 299 | RPL23AP94, TAP1, ARHGEF16, NPC2 |
| 300 | RPL23AP94, TAP1, MICB, TMCO4 |
| 301 | RPL23AP94, TAP1, MICB, RAPGEF3 |
| 302 | RPL23AP94, TAP1, MICB, NPC2 |
| 303 | RPL23AP94, TAP1, TMCO4, RAPGEF3 |
| 304 | RPL23AP94, TAP1, TMCO4, NPC2 |
| 305 | RPL23AP94, TAP1, RAPGEF3, NPC2 |
| 306 | RPL23AP94, PLCG2, ARHGEF35, ARHGEF16 |
| 307 | RPL23AP94, PLCG2, ARHGEF35, MICB |
| 308 | RPL23AP94, PLCG2, ARHGEF35, TMCO4 |
| 309 | RPL23AP94, PLCG2, ARHGEF35, RAPGEF3 |
| 310 | RPL23AP94, PLCG2, ARHGEF35, NPC2 |
| 311 | RPL23AP94, PLCG2, ARHGEF16, MICB |
| 312 | RPL23AP94, PLCG2, ARHGEF16, TMCO4 |
| 313 | RPL23AP94, PLCG2, ARHGEF16, RAPGEF3 |
| 314 | RPL23AP94, PLCG2, ARHGEF16, NPC2 |
| 315 | RPL23AP94, PLCG2, MICB, TMCO4 |
| 316 | RPL23AP94, PLCG2, MICB, RAPGEF3 |
| 317 | RPL23AP94, PLCG2, MICB, NPC2 |
| 318 | RPL23AP94, PLCG2, TMCO4, RAPGEF3 |
| 319 | RPL23AP94, PLCG2, TMCO4, NPC2 |
| 320 | RPL23AP94, PLCG2, RAPGEF3, NPC2 |
| 321 | RPL23AP94, ARHGEF35, ARHGEF16, MICB |
| 322 | RPL23AP94, ARHGEF35, ARHGEF16, TMCO4 |
| 323 | RPL23AP94, ARHGEF35, ARHGEF16, RAPGEF3 |
| 324 | RPL23AP94, ARHGEF35, ARHGEF16, NPC2 |
| 325 | RPL23AP94, ARHGEF35, MICB, TMCO4 |
| 326 | RPL23AP94, ARHGEF35, MICB, RAPGEF3 |
| 327 | RPL23AP94, ARHGEF35, MICB, NPC2 |
| 328 | RPL23AP94, ARHGEF35, TMCO4, RAPGEF3 |
| 329 | RPL23AP94, ARHGEF35, TMCO4, NPC2 |
| 330 | RPL23AP94, ARHGEF35, RAPGEF3, NPC2 |
| 331 | RPL23AP94, ARHGEF16, MICB, TMCO4 |
| 332 | RPL23AP94, ARHGEF16, MICB, RAPGEF3 |
| 333 | RPL23AP94, ARHGEF16, MICB, NPC2 |
| 334 | RPL23AP94, ARHGEF16, TMCO4, RAPGEF3 |
| 335 | RPL23AP94, ARHGEF16, TMCO4, NPC2 |
| 336 | RPL23AP94, ARHGEF16, RAPGEF3, NPC2 |
| 337 | RPL23AP94, MICB, TMCO4, RAPGEF3 |
| 338 | RPL23AP94, MICB, TMCO4, NPC2 |
| 339 | RPL23AP94, MICB, RAPGEF3, NPC2 |
| 340 | RPL23AP94, TMCO4, RAPGEF3, NPC2 |
| 341 | SYN2, NSMF, TAP1, PLCG2 |
| 342 | SYN2, NSMF, TAP1, ARHGEF35 |
| 343 | SYN2, NSMF, TAP1, ARHGEF16 |
| 344 | SYN2, NSMF, TAP1, MICB |
| 345 | SYN2, NSMF, TAP1, TMCO4 |
| 346 | SYN2, NSMF, TAP1, RAPGEF3 |
| 347 | SYN2, NSMF, TAP1, NPC2 |
| 348 | SYN2, NSMF, PLCG2, ARHGEF35 |
| 349 | SYN2, NSMF, PLCG2, ARHGEF16 |
| 350 | SYN2, NSMF, PLCG2, MICB |
| 351 | SYN2, NSMF, PLCG2, TMCO4 |
| 352 | SYN2, NSMF, PLCG2, RAPGEF3 |
| 353 | SYN2, NSMF, PLCG2, NPC2 |
| 354 | SYN2, NSMF, ARHGEF35, ARHGEF16 |
| 355 | SYN2, NSMF, ARHGEF35, MICB |
| 356 | SYN2, NSMF, ARHGEF35, TMCO4 |
| 357 | SYN2, NSMF, ARHGEF35, RAPGEF3 |
| 358 | SYN2, NSMF, ARHGEF35, NPC2 |
| 359 | SYN2, NSMF, ARHGEF16, MICB |
| 360 | SYN2, NSMF, ARHGEF16, TMCO4 |
| 361 | SYN2, NSMF, ARHGEF16, RAPGEF3 |
| 362 | SYN2, NSMF, ARHGEF16, NPC2 |
| 363 | SYN2, NSMF, MICB, TMCO4 |
| 364 | SYN2, NSMF, MICB, RAPGEF3 |
| 365 | SYN2, NSMF, MICB, NPC2 |
| 366 | SYN2, NSMF, TMCO4, RAPGEF3 |
| 367 | SYN2, NSMF, TMCO4, NPC2 |
| 368 | SYN2, NSMF, RAPGEF3, NPC2 |
| 369 | SYN2, TAP1, PLCG2, ARHGEF35 |
| 370 | SYN2, TAP1, PLCG2, ARHGEF16 |
| 371 | SYN2, TAP1, PLCG2, MICB |
| 372 | SYN2, TAP1, PLCG2, TMCO4 |
| 373 | SYN2, TAP1, PLCG2, RAPGEF3 |
| 374 | SYN2, TAP1, PLCG2, NPC2 |
| 375 | SYN2, TAP1, ARHGEF35, ARHGEF16 |
| 376 | SYN2, TAP1, ARHGEF35, MICB |
| 377 | SYN2, TAP1, ARHGEF35, TMCO4 |
| 378 | SYN2, TAP1, ARHGEF35, RAPGEF3 |
| 379 | SYN2, TAP1, ARHGEF35, NPC2 |
| 380 | SYN2, TAP1, ARHGEF16, MICB |
| 381 | SYN2, TAP1, ARHGEF16, TMCO4 |
| 382 | SYN2, TAP1, ARHGEF16, RAPGEF3 |
| 383 | SYN2, TAP1, ARHGEF16, NPC2 |
| 384 | SYN2, TAP1, MICB, TMCO4 |
| 385 | SYN2, TAP1, MICB, RAPGEF3 |
| 386 | SYN2, TAP1, MICB, NPC2 |
| 387 | SYN2, TAP1, TMCO4, RAPGEF3 |
| 388 | SYN2, TAP1, TMCO4, NPC2 |
| 389 | SYN2, TAP1, RAPGEF3, NPC2 |
| 390 | SYN2, PLCG2, ARHGEF35, ARHGEF16 |
| 391 | SYN2, PLCG2, ARHGEF35, MICB |
| 392 | SYN2, PLCG2, ARHGEF35, TMCO4 |
| 393 | SYN2, PLCG2, ARHGEF35, RAPGEF3 |
| 394 | SYN2, PLCG2, ARHGEF35, NPC2 |
| 395 | SYN2, PLCG2, ARHGEF16, MICB |
| 396 | SYN2, PLCG2, ARHGEF16, TMCO4 |
| 397 | SYN2, PLCG2, ARHGEF16, RAPGEF3 |
| 398 | SYN2, PLCG2, ARHGEF16, NPC2 |
| 399 | SYN2, PLCG2, MICB, TMCO4 |
| 400 | SYN2, PLCG2, MICB, RAPGEF3 |
| 401 | SYN2, PLCG2, MICB, NPC2 |
| 402 | SYN2, PLCG2, TMCO4, RAPGEF3 |
| 403 | SYN2, PLCG2, TMCO4, NPC2 |
| 404 | SYN2, PLCG2, RAPGEF3, NPC2 |
| 405 | SYN2, ARHGEF35, ARHGEF16, MICB |
| 406 | SYN2, ARHGEF35, ARHGEF16, TMCO4 |
| 407 | SYN2, ARHGEF35, ARHGEF16, RAPGEF3 |
| 408 | SYN2, ARHGEF35, ARHGEF16, NPC2 |
| 409 | SYN2, ARHGEF35, MICB, TMCO4 |
| 410 | SYN2, ARHGEF35, MICB, RAPGEF3 |
| 411 | SYN2, ARHGEF35, MICB, NPC2 |
| 412 | SYN2, ARHGEF35, TMCO4, RAPGEF3 |
| 413 | SYN2, ARHGEF35, TMCO4, NPC2 |
| 414 | SYN2, ARHGEF35, RAPGEF3, NPC2 |
| 415 | SYN2, ARHGEF16, MICB, TMCO4 |
| 416 | SYN2, ARHGEF16, MICB, RAPGEF3 |
| 417 | SYN2, ARHGEF16, MICB, NPC2 |
| 418 | SYN2, ARHGEF16, TMCO4, RAPGEF3 |
| 419 | SYN2, ARHGEF16, TMCO4, NPC2 |
| 420 | SYN2, ARHGEF16, RAPGEF3, NPC2 |
| 421 | SYN2, MICB, TMCO4, RAPGEF3 |
| 422 | SYN2, MICB, TMCO4, NPC2 |
| 423 | SYN2, MICB, RAPGEF3, NPC2 |
| 424 | SYN2, TMCO4, RAPGEF3, NPC2 |
| 425 | NSMF, TAP1, PLCG2, ARHGEF35 |
| 426 | NSMF, TAP1, PLCG2, ARHGEF16 |
| 427 | NSMF, TAP1, PLCG2, MICB |
| 428 | NSMF, TAP1, PLCG2, TMCO4 |
| 429 | NSMF, TAP1, PLCG2, RAPGEF3 |
| 430 | NSMF, TAP1, PLCG2, NPC2 |
| 431 | NSMF, TAP1, ARHGEF35, ARHGEF16 |
| 432 | NSMF, TAP1, ARHGEF35, MICB |
| 433 | NSMF, TAP1, ARHGEF35, TMCO4 |
| 434 | NSMF, TAP1, ARHGEF35, RAPGEF3 |
| 435 | NSMF, TAP1, ARHGEF35, NPC2 |
| 436 | NSMF, TAP1, ARHGEF16, MICB |
| 437 | NSMF, TAP1, ARHGEF16, TMCO4 |
| 438 | NSMF, TAP1, ARHGEF16, RAPGEF3 |
| 439 | NSMF, TAP1, ARHGEF16, NPC2 |
| 440 | NSMF, TAP1, MICB, TMCO4 |
| 441 | NSMF, TAP1, MICB, RAPGEF3 |
| 442 | NSMF, TAP1, MICB, NPC2 |
| 443 | NSMF, TAP1, TMCO4, RAPGEF3 |
| 444 | NSMF, TAP1, TMCO4, NPC2 |
| 445 | NSMF, TAP1, RAPGEF3, NPC2 |
| 446 | NSMF, PLCG2, ARHGEF35, ARHGEF16 |
| 447 | NSMF, PLCG2, ARHGEF35, MICB |
| 448 | NSMF, PLCG2, ARHGEF35, TMCO4 |
| 449 | NSMF, PLCG2, ARHGEF35, RAPGEF3 |
| 450 | NSMF, PLCG2, ARHGEF35, NPC2 |
| 451 | NSMF, PLCG2, ARHGEF16, MICB |
| 452 | NSMF, PLCG2, ARHGEF16, TMCO4 |
| 453 | NSMF, PLCG2, ARHGEF16, RAPGEF3 |
| 454 | NSMF, PLCG2, ARHGEF16, NPC2 |
| 455 | NSMF, PLCG2, MICB, TMCO4 |
| 456 | NSMF, PLCG2, MICB, RAPGEF3 |
| 457 | NSMF, PLCG2, MICB, NPC2 |
| 458 | NSMF, PLCG2, TMCO4, RAPGEF3 |
| 459 | NSMF, PLCG2, TMCO4, NPC2 |
| 460 | NSMF, PLCG2, RAPGEF3, NPC2 |
| 461 | NSMF, ARHGEF35, ARHGEF16, MICB |
| 462 | NSMF, ARHGEF35, ARHGEF16, TMCO4 |
| 463 | NSMF, ARHGEF35, ARHGEF16, RAPGEF3 |
| 464 | NSMF, ARHGEF35, ARHGEF16, NPC2 |
| 465 | NSMF, ARHGEF35, MICB, TMCO4 |
| 466 | NSMF, ARHGEF35, MICB, RAPGEF3 |
| 467 | NSMF, ARHGEF35, MICB, NPC2 |
| 468 | NSMF, ARHGEF35, TMCO4, RAPGEF3 |
| 469 | NSMF, ARHGEF35, TMCO4, NPC2 |
| 470 | NSMF, ARHGEF35, RAPGEF3, NPC2 |
| 471 | NSMF, ARHGEF16, MICB, TMCO4 |
| 472 | NSMF, ARHGEF16, MICB, RAPGEF3 |
| 473 | NSMF, ARHGEF16, MICB, NPC2 |
| 474 | NSMF, ARHGEF16, TMCO4, RAPGEF3 |
| 475 | NSMF, ARHGEF16, TMCO4, NPC2 |
| 476 | NSMF, ARHGEF16, RAPGEF3, NPC2 |
| 477 | NSMF, MICB, TMCO4, RAPGEF3 |
| 478 | NSMF, MICB, TMCO4, NPC2 |
| 479 | NSMF, MICB, RAPGEF3, NPC2 |
| 480 | NSMF, TMCO4, RAPGEF3, NPC2 |
| 481 | TAP1, PLCG2, ARHGEF35, ARHGEF16 |
| 482 | TAP1, PLCG2, ARHGEF35, MICB |
| 483 | TAP1, PLCG2, ARHGEF35, TMCO4 |
| 484 | TAP1, PLCG2, ARHGEF35, RAPGEF3 |
| 485 | TAP1, PLCG2, ARHGEF35, NPC2 |
| 486 | TAP1, PLCG2, ARHGEF16, MICB |
| 487 | TAP1, PLCG2, ARHGEF16, TMCO4 |
| 488 | TAP1, PLCG2, ARHGEF16, RAPGEF3 |
| 489 | TAP1, PLCG2, ARHGEF16, NPC2 |
| 490 | TAP1, PLCG2, MICB, TMCO4 |
| 491 | TAP1, PLCG2, MICB, RAPGEF3 |
| 492 | TAP1, PLCG2, MICB, NPC2 |
| 493 | TAP1, PLCG2, TMCO4, RAPGEF3 |
| 494 | TAP1, PLCG2, TMCO4, NPC2 |
| 495 | TAP1, PLCG2, RAPGEF3, NPC2 |
| 496 | TAP1, ARHGEF35, ARHGEF16, MICB |
| 497 | TAP1, ARHGEF35, ARHGEF16, TMCO4 |
| 498 | TAP1, ARHGEF35, ARHGEF16, RAPGEF3 |
| 499 | TAP1, ARHGEF35, ARHGEF16, NPC2 |
| 500 | TAP1, ARHGEF35, MICB, TMCO4 |
| 501 | TAP1, ARHGEF35, MICB, RAPGEF3 |
| 502 | TAP1, ARHGEF35, MICB, NPC2 |
| 503 | TAP1, ARHGEF35, TMCO4, RAPGEF3 |
| 504 | TAP1, ARHGEF35, TMCO4, NPC2 |
| 505 | TAP1, ARHGEF35, RAPGEF3, NPC2 |
| 506 | TAP1, ARHGEF16, MICB, TMCO4 |
| 507 | TAP1, ARHGEF16, MICB, RAPGEF3 |
| 508 | TAP1, ARHGEF16, MICB, NPC2 |
| 509 | TAP1, ARHGEF16, TMCO4, RAPGEF3 |
| 510 | TAP1, ARHGEF16, TMCO4, NPC2 |
| 511 | TAP1, ARHGEF16, RAPGEF3, NPC2 |
| 512 | TAP1, MICB, TMCO4, RAPGEF3 |
| 513 | TAP1, MICB, TMCO4, NPC2 |
| 514 | TAP1, MICB, RAPGEF3, NPC2 |
| 515 | TAP1, TMCO4, RAPGEF3, NPC2 |
| 516 | PLCG2, ARHGEF35, ARHGEF16, MICB |
| 517 | PLCG2, ARHGEF35, ARHGEF16, TMCO4 |
| 518 | PLCG2, ARHGEF35, ARHGEF16, RAPGEF3 |
| 519 | PLCG2, ARHGEF35, ARHGEF16, NPC2 |
| 520 | PLCG2, ARHGEF35, MICB, TMCO4 |
| 521 | PLCG2, ARHGEF35, MICB, RAPGEF3 |
| 522 | PLCG2, ARHGEF35, MICB, NPC2 |
| 523 | PLCG2, ARHGEF35, TMCO4, RAPGEF3 |
| 524 | PLCG2, ARHGEF35, TMCO4, NPC2 |
| 525 | PLCG2, ARHGEF35, RAPGEF3, NPC2 |
| 526 | PLCG2, ARHGEF16, MICB, TMCO4 |
| 527 | PLCG2, ARHGEF16, MICB, RAPGEF3 |
| 528 | PLCG2, ARHGEF16, MICB, NPC2 |
| 529 | PLCG2, ARHGEF16, TMCO4, RAPGEF3 |
| 530 | PLCG2, ARHGEF16, TMCO4, NPC2 |
| 531 | PLCG2, ARHGEF16, RAPGEF3, NPC2 |
| 532 | PLCG2, MICB, TMCO4, RAPGEF3 |
| 533 | PLCG2, MICB, TMCO4, NPC2 |
| 534 | PLCG2, MICB, RAPGEF3, NPC2 |
| 535 | PLCG2, TMCO4, RAPGEF3, NPC2 |
| 536 | ARHGEF35, ARHGEF16, MICB, TMCO4 |
| 537 | ARHGEF35, ARHGEF16, MICB, RAPGEF3 |
| 538 | ARHGEF35, ARHGEF16, MICB, NPC2 |
| 539 | ARHGEF35, ARHGEF16, TMCO4, RAPGEF3 |
| 540 | ARHGEF35, ARHGEF16, TMCO4, NPC2 |
| 541 | ARHGEF35, ARHGEF16, RAPGEF3, NPC2 |
| 542 | ARHGEF35, MICB, TMCO4, RAPGEF3 |
| 543 | ARHGEF35, MICB, TMCO4, NPC2 |
| 544 | ARHGEF35, MICB, RAPGEF3, NPC2 |
| 545 | ARHGEF35, TMCO4, RAPGEF3, NPC2 |
| 546 | ARHGEF16, MICB, TMCO4, RAPGEF3 |
| 547 | ARHGEF16, MICB, TMCO4, NPC2 |
| 548 | ARHGEF16, MICB, RAPGEF3, NPC2 |
| 549 | ARHGEF16, TMCO4, RAPGEF3, NPC2 |
| 550 | MICB, TMCO4, RAPGEF3, NPC2 |
| 551 | RPL23AP94, SYN2, NSMF, TAP1, PLCG2 |
| 552 | RPL23AP94, SYN2, NSMF, TAP1, ARHGEF35 |
| 553 | RPL23AP94, SYN2, NSMF, TAP1, ARHGEF16 |
| 554 | RPL23AP94, SYN2, NSMF, TAP1, MICB |
| 555 | RPL23AP94, SYN2, NSMF, TAP1, TMCO4 |
| 556 | RPL23AP94, SYN2, NSMF, TAP1, RAPGEF3 |
| 557 | RPL23AP94, SYN2, NSMF, TAP1, NPC2 |
| 558 | RPL23AP94, SYN2, NSMF, PLCG2, ARHGEF35 |
| 559 | RPL23AP94, SYN2, NSMF, PLCG2, ARHGEF16 |
| 560 | RPL23AP94, SYN2, NSMF, PLCG2, MICB |
| 561 | RPL23AP94, SYN2, NSMF, PLCG2, TMCO4 |
| 562 | RPL23AP94, SYN2, NSMF, PLCG2, RAPGEF3 |
| 563 | RPL23AP94, SYN2, NSMF, PLCG2, NPC2 |
| 564 | RPL23AP94, SYN2, NSMF, ARHGEF35, ARHGEF16 |
| 565 | RPL23AP94, SYN2, NSMF, ARHGEF35, MICB |
| 566 | RPL23AP94, SYN2, NSMF, ARHGEF35, TMCO4 |
| 567 | RPL23AP94, SYN2, NSMF, ARHGEF35, RAPGEF3 |
| 568 | RPL23AP94, SYN2, NSMF, ARHGEF35, NPC2 |
| 569 | RPL23AP94, SYN2, NSMF, ARHGEF16, MICB |
| 570 | RPL23AP94, SYN2, NSMF, ARHGEF16, TMCO4 |
| 571 | RPL23AP94, SYN2, NSMF, ARHGEF16, RAPGEF3 |
| 572 | RPL23AP94, SYN2, NSMF, ARHGEF16, NPC2 |
| 573 | RPL23AP94, SYN2, NSMF, MICB, TMCO4 |
| 574 | RPL23AP94, SYN2, NSMF, MICB, RAPGEF3 |
| 575 | RPL23AP94, SYN2, NSMF, MICB, NPC2 |
| 576 | RPL23AP94, SYN2, NSMF, TMCO4, RAPGEF3 |
| 577 | RPL23AP94, SYN2, NSMF, TMCO4, NPC2 |
| 578 | RPL23AP94, SYN2, NSMF, RAPGEF3, NPC2 |
| 579 | RPL23AP94, SYN2, TAP1, PLCG2, ARHGEF35 |
| 580 | RPL23AP94, SYN2, TAP1, PLCG2, ARHGEF16 |
| 581 | RPL23AP94, SYN2, TAP1, PLCG2, MICB |
| 582 | RPL23AP94, SYN2, TAP1, PLCG2, TMCO4 |
| 583 | RPL23AP94, SYN2, TAP1, PLCG2, RAPGEF3 |
| 584 | RPL23AP94, SYN2, TAP1, PLCG2, NPC2 |
| 585 | RPL23AP94, SYN2, TAP1, ARHGEF35, ARHGEF16 |
| 586 | RPL23AP94, SYN2, TAP1, ARHGEF35, MICB |
| 587 | RPL23AP94, SYN2, TAP1, ARHGEF35, TMCO4 |
| 588 | RPL23AP94, SYN2, TAP1, ARHGEF35, RAPGEF3 |
| 589 | RPL23AP94, SYN2, TAP1, ARHGEF35, NPC2 |
| 590 | RPL23AP94, SYN2, TAP1, ARHGEF16, MICB |
| 591 | RPL23AP94, SYN2, TAP1, ARHGEF16, TMCO4 |
| 592 | RPL23AP94, SYN2, TAP1, ARHGEF16, RAPGEF3 |
| 593 | RPL23AP94, SYN2, TAPI, ARHGEF16, NPC2 |
| 594 | RPL23AP94, SYN2, TAP1, MICB, TMCO4 |
| 595 | RPL23AP94, SYN2, TAP1, MICB, RAPGEF3 |
| 596 | RPL23AP94, SYN2, TAP1, MICB, NPC2 |
| 597 | RPL23AP94, SYN2, TAP1, TMCO4, RAPGEF3 |
| 598 | RPL23AP94, SYN2, TAP1, TMCO4, NPC2 |
| 599 | RPL23AP94, SYN2, TAP1, RAPGEF3, NPC2 |
| 600 | RPL23AP94, SYN2, PLCG2, ARHGEF35, ARHGEF16 |
| 601 | RPL23AP94, SYN2, PLCG2, ARHGEF35, MICB |
| 602 | RPL23AP94, SYN2, PLCG2, ARHGEF35, TMCO4 |
| 603 | RPL23AP94, SYN2, PLCG2, ARHGEF35, RAPGEF3 |
| 604 | RPL23AP94, SYN2, PLCG2, ARHGEF35, NPC2 |
| 605 | RPL23AP94, SYN2, PLCG2, ARHGEF16, MICB |
| 606 | RPL23AP94, SYN2, PLCG2, ARHGEF16, TMCO4 |
| 607 | RPL23AP94, SYN2, PLCG2, ARHGEF16, RAPGEF3 |
| 608 | RPL23AP94, SYN2, PLCG2, ARHGEF16, NPC2 |
| 609 | RPL23AP94, SYN2, PLCG2, MICB, TMCO4 |
| 610 | RPL23AP94, SYN2, PLCG2, MICB, RAPGEF3 |
| 611 | RPL23AP94, SYN2, PLCG2, MICB, NPC2 |
| 612 | RPL23AP94, SYN2, PLCG2, TMCO4, RAPGEF3 |
| 613 | RPL23AP94, SYN2, PLCG2, TMCO4, NPC2 |
| 614 | RPL23AP94, SYN2, PLCG2, RAPGEF3, NPC2 |
| 615 | RPL23AP94, SYN2, ARHGEF35, ARHGEF16, MICB |
| 616 | RPL23AP94, SYN2, ARHGEF35, ARHGEF16, TMCO4 |
| 617 | RPL23AP94, SYN2, ARHGEF35, ARHGEF16, RAPGEF3 |
| 618 | RPL23AP94, SYN2, ARHGEF35, ARHGEF16, NPC2 |
| 619 | RPL23AP94, SYN2, ARHGEF35, MICB, TMCO4 |
| 620 | RPL23AP94, SYN2, ARHGEF35, MICB, RAPGEF3 |
| 621 | RPL23AP94, SYN2, ARHGEF35, MICB, NPC2 |
| 622 | RPL23AP94, SYN2, ARHGEF35, TMCO4, RAPGEF3 |
| 623 | RPL23AP94, SYN2, ARHGEF35, TMCO4, NPC2 |
| 624 | RPL23AP94, SYN2, ARHGEF35, RAPGEF3, NPC2 |
| 625 | RPL23AP94, SYN2, ARHGEF16, MICB, TMCO4 |
| 626 | RPL23AP94, SYN2, ARHGEF16, MICB, RAPGEF3 |
| 627 | RPL23AP94, SYN2, ARHGEF16, MICB, NPC2 |
| 628 | RPL23AP94, SYN2, ARHGEF16, TMCO4, RAPGEF3 |
| 629 | RPL23AP94, SYN2, ARHGEF16, TMCO4, NPC2 |
| 630 | RPL23AP94, SYN2, ARHGEF16, RAPGEF3, NPC2 |
| 631 | RPL23AP94, SYN2, MICB, TMCO4, RAPGEF3 |
| 632 | RPL23AP94, SYN2, MICB, TMCO4, NPC2 |
| 633 | RPL23AP94, SYN2, MICB, RAPGEF3, NPC2 |
| 634 | RPL23AP94, SYN2, TMCO4, RAPGEF3, NPC2 |
| 635 | RPL23AP94, NSMF, TAP1, PLCG2, ARHGEF35 |
| 636 | RPL23AP94, NSMF, TAP1, PLCG2, ARHGEF16 |
| 637 | RPL23AP94, NSMF, TAP1, PLCG2, MICB |
| 638 | RPL23AP94, NSMF, TAP1, PLCG2, TMCO4 |
| 639 | RPL23AP94, NSMF, TAP1, PLCG2, RAPGEF3 |
| 640 | RPL23AP94, NSMF, TAP1, PLCG2, NPC2 |
| 641 | RPL23AP94, NSMF, TAP1, ARHGEF35, ARHGEF16 |
| 642 | RPL23AP94, NSMF, TAP1, ARHGEF35, MICB |
| 643 | RPL23AP94, NSMF, TAP1, ARHGEF35, TMCO4 |
| 644 | RPL23AP94, NSMF, TAP1, ARHGEF35, RAPGEF3 |
| 645 | RPL23AP94, NSMF, TAP1, ARHGEF35, NPC2 |
| 646 | RPL23AP94, NSMF, TAP1, ARHGEF16, MICB |
| 647 | RPL23AP94, NSMF, TAP1, ARHGEF16, TMCO4 |
| 648 | RPL23AP94, NSMF, TAP1, ARHGEF16, RAPGEF3 |
| 649 | RPL23AP94, NSMF, TAP1, ARHGEF16, NPC2 |
| 650 | RPL23AP94, NSMF, TAP1, MICB, TMCO4 |
| 651 | RPL23AP94, NSMF, TAP1, MICB, RAPGEF3 |
| 652 | RPL23AP94, NSMF, TAP1, MICB, NPC2 |
| 653 | RPL23AP94, NSMF, TAP1, TMCO4, RAPGEF3 |
| 654 | RPL23AP94, NSMF, TAP1, TMCO4, NPC2 |
| 655 | RPL23AP94, NSMF, TAP1, RAPGEF3, NPC2 |
| 656 | RPL23AP94, NSMF, PLCG2, ARHGEF35, ARHGEF16 |
| 657 | RPL23AP94, NSMF, PLCG2, ARHGEF35, MICB |
| 658 | RPL23AP94, NSMF, PLCG2, ARHGEF35, TMCO4 |
| 659 | RPL23AP94, NSMF, PLCG2, ARHGEF35, RAPGEF3 |
| 660 | RPL23AP94, NSMF, PLCG2, ARHGEF35, NPC2 |
| 661 | RPL23AP94, NSMF, PLCG2, ARHGEF16, MICB |
| 662 | RPL23AP94, NSMF, PLCG2, ARHGEF16, TMCO4 |
| 663 | RPL23AP94, NSMF, PLCG2, ARHGEF16, RAPGEF3 |
| 664 | RPL23AP94, NSMF, PLCG2, ARHGEF16, NPC2 |
| 665 | RPL23AP94, NSMF, PLCG2, MICB, TMCO4 |
| 666 | RPL23AP94, NSMF, PLCG2, MICB, RAPGEF3 |
| 667 | RPL23AP94, NSMF, PLCG2, MICB, NPC2 |
| 668 | RPL23AP94, NSMF, PLCG2, TMCO4, RAPGEF3 |
| 669 | RPL23AP94, NSMF, PLCG2, TMCO4, NPC2 |
| 670 | RPL23AP94, NSMF, PLCG2, RAPGEF3, NPC2 |
| 671 | RPL23AP94, NSMF, ARHGEF35, ARHGEF16, MICB |
| 672 | RPL23AP94, NSMF, ARHGEF35, ARHGEF16, TMCO4 |
| 673 | RPL23AP94, NSMF, ARHGEF35, ARHGEF16, RAPGEF3 |
| 674 | RPL23AP94, NSMF, ARHGEF35, ARHGEF16, NPC2 |
| 675 | RPL23AP94, NSMF, ARHGEF35, MICB, TMCO4 |
| 676 | RPL23AP94, NSMF, ARHGEF35, MICB, RAPGEF3 |
| 677 | RPL23AP94, NSMF, ARHGEF35, MICB, NPC2 |
| 678 | RPL23AP94, NSMF, ARHGEF35, TMCO4, RAPGEF3 |
| 679 | RPL23AP94, NSMF, ARHGEF35, TMCO4, NPC2 |
| 680 | RPL23AP94, NSMF, ARHGEF35, RAPGEF3, NPC2 |
| 681 | RPL23AP94, NSMF, ARHGEF16, MICB, TMCO4 |
| 682 | RPL23AP94, NSMF, ARHGEF16, MICB, RAPGEF3 |
| 683 | RPL23AP94, NSMF, ARHGEF16, MICB, NPC2 |
| 684 | RPL23AP94, NSMF, ARHGEF16, TMCO4, RAPGEF3 |
| 685 | RPL23AP94, NSMF, ARHGEF16, TMCO4, NPC2 |
| 686 | RPL23AP94, NSMF, ARHGEF16, RAPGEF3, NPC2 |
| 687 | RPL23AP94, NSMF, MICB, TMCO4, RAPGEF3 |
| 688 | RPL23AP94, NSMF, MICB, TMCO4, NPC2 |
| 689 | RPL23AP94, NSMF, MICB, RAPGEF3, NPC2 |
| 690 | RPL23AP94, NSMF, TMCO4, RAPGEF3, NPC2 |
| 691 | RPL23AP94, TAP1, PLCG2, ARHGEF35, ARHGEF16 |
| 692 | RPL23AP94, TAP1, PLCG2, ARHGEF35, MICB |
| 693 | RPL23AP94, TAP1, PLCG2, ARHGEF35, TMCO4 |
| 694 | RPL23AP94, TAP1, PLCG2, ARHGEF35, RAPGEF3 |
| 695 | RPL23AP94, TAP1, PLCG2, ARHGEF35, NPC2 |
| 696 | RPL23AP94, TAP1, PLCG2, ARHGEF16, MICB |
| 697 | RPL23AP94, TAP1, PLCG2, ARHGEF16, TMCO4 |
| 698 | RPL23AP94, TAP1, PLCG2, ARHGEF16, RAPGEF3 |
| 699 | RPL23AP94, TAP1, PLCG2, ARHGEF16, NPC2 |
| 700 | RPL23AP94, TAP1, PLCG2, MICB, TMCO4 |
| 701 | RPL23AP94, TAP1, PLCG2, MICB, RAPGEF3 |
| 702 | RPL23AP94, TAP1, PLCG2, MICB, NPC2 |
| 703 | RPL23AP94, TAP1, PLCG2, TMCO4, RAPGEF3 |
| 704 | RPL23AP94, TAP1, PLCG2, TMCO4, NPC2 |
| 705 | RPL23AP94, TAP1, PLCG2, RAPGEF3, NPC2 |
| 706 | RPL23AP94, TAP1, ARHGEF35, ARHGEF16, MICB |
| 707 | RPL23AP94, TAP1, ARHGEF35, ARHGEF16, TMCO4 |
| 708 | RPL23AP94, TAP1, ARHGEF35, ARHGEF16, RAPGEF3 |
| 709 | RPL23AP94, TAP1, ARHGEF35, ARHGEF16, NPC2 |
| 710 | RPL23AP94, TAP1, ARHGEF35, MICB, TMCO4 |
| 711 | RPL23AP94, TAP1, ARHGEF35, MICB, RAPGEF3 |
| 712 | RPL23AP94, TAP1, ARHGEF35, MICB, NPC2 |
| 713 | RPL23AP94, TAP1, ARHGEF35, TMCO4, RAPGEF3 |
| 714 | RPL23AP94, TAP1, ARHGEF35, TMCO4, NPC2 |
| 715 | RPL23AP94, TAP1, ARHGEF35, RAPGEF3, NPC2 |
| 716 | RPL23AP94, TAP1, ARHGEF16, MICB, TMCO4 |
| 717 | RPL23AP94, TAP1, ARHGEF16, MICB, RAPGEF3 |
| 718 | RPL23AP94, TAP1, ARHGEF16, MICB, NPC2 |
| 719 | RPL23AP94, TAP1, ARHGEF16, TMCO4, RAPGEF3 |
| 720 | RPL23AP94, TAP1, ARHGEF16, TMCO4, NPC2 |
| 721 | RPL23AP94, TAP1, ARHGEF16, RAPGEF3, NPC2 |
| 722 | RPL23AP94, TAP1, MICB, TMCO4, RAPGEF3 |
| 723 | RPL23AP94, TAP1, MICB, TMCO4, NPC2 |
| 724 | RPL23AP94, TAP1, MICB, RAPGEF3, NPC2 |
| 725 | RPL23AP94, TAP1, TMCO4, RAPGEF3, NPC2 |
| 726 | RPL23AP94, PLCG2, ARHGEF35, ARHGEF16, MICB |
| 727 | RPL23AP94, PLCG2, ARHGEF35, ARHGEF16, TMCO4 |
| 728 | RPL23AP94, PLCG2, ARHGEF35, ARHGEF16, RAPGEF3 |
| 729 | RPL23AP94, PLCG2, ARHGEF35, ARHGEF16, NPC2 |
| 730 | RPL23AP94, PLCG2, ARHGEF35, MICB, TMCO4 |
| 731 | RPL23AP94, PLCG2, ARHGEF35, MICB, RAPGEF3 |
| 732 | RPL23AP94, PLCG2, ARHGEF35, MICB, NPC2 |
| 733 | RPL23AP94, PLCG2, ARHGEF35, TMCO4, RAPGEF3 |
| 734 | RPL23AP94, PLCG2, ARHGEF35, TMCO4, NPC2 |
| 735 | RPL23AP94, PLCG2, ARHGEF35, RAPGEF3, NPC2 |
| 736 | RPL23AP94, PLCG2, ARHGEF16, MICB, TMCO4 |
| 737 | RPL23AP94, PLCG2, ARHGEF16, MICB, RAPGEF3 |
| 738 | RPL23AP94, PLCG2, ARHGEF16, MICB, NPC2 |
| 739 | RPL23AP94, PLCG2, ARHGEF16, TMCO4, RAPGEF3 |
| 740 | RPL23AP94, PLCG2, ARHGEF16, TMCO4, NPC2 |
| 741 | RPL23AP94, PLCG2, ARHGEF16, RAPGEF3, NPC2 |
| 742 | RPL23AP94, PLCG2, MICB, TMCO4, RAPGEF3 |
| 743 | RPL23AP94, PLCG2, MICB, TMCO4, NPC2 |
| 744 | RPL23AP94, PLCG2, MICB, RAPGEF3, NPC2 |
| 745 | RPL23AP94, PLCG2, TMCO4, RAPGEF3, NPC2 |
| 746 | RPL23AP94, ARHGEF35, ARHGEF16, MICB, TMCO4 |
| 747 | RPL23AP94, ARHGEF35, ARHGEF16, MICB, RAPGEF3 |
| 748 | RPL23AP94, ARHGEF35, ARHGEF16, MICB, NPC2 |
| 749 | RPL23AP94, ARHGEF35, ARHGEF16, TMCO4, RAPGEF3 |
| 750 | RPL23AP94, ARHGEF35, ARHGEF16, TMCO4, NPC2 |
| 751 | RPL23AP94, ARHGEF35, ARHGEF16, RAPGEF3, NPC2 |
| 752 | RPL23AP94, ARHGEF35, MICB, TMCO4, RAPGEF3 |
| 753 | RPL23AP94, ARHGEF35, MICB, TMCO4, NPC2 |
| 754 | RPL23AP94, ARHGEF35, MICB, RAPGEF3, NPC2 |
| 755 | RPL23AP94, ARHGEF35, TMCO4, RAPGEF3, NPC2 |
| 756 | RPL23AP94, ARHGEF16, MICB, TMCO4, RAPGEF3 |
| 757 | RPL23AP94, ARHGEF16, MICB, TMCO4, NPC2 |
| 758 | RPL23AP94, ARHGEF16, MICB, RAPGEF3, NPC2 |
| 759 | RPL23AP94, ARHGEF16, TMCO4, RAPGEF3, NPC2 |
| 760 | RPL23AP94, MICB, TMCO4, RAPGEF3, NPC2 |
| 761 | SYN2, NSMF, TAP1, PLCG2, ARHGEF35 |
| 762 | SYN2, NSMF, TAP1, PLCG2, ARHGEF16 |
| 763 | SYN2, NSMF, TAP1, PLCG2, MICB |
| 764 | SYN2, NSMF, TAP1, PLCG2, TMCO4 |
| 765 | SYN2, NSMF, TAP1, PLCG2, RAPGEF3 |
| 766 | SYN2, NSMF, TAP1, PLCG2, NPC2 |
| 767 | SYN2, NSMF, TAP1, ARHGEF35, ARHGEF16 |
| 768 | SYN2, NSMF, TAP1, ARHGEF35, MICB |
| 769 | SYN2, NSMF, TAP1, ARHGEF35, TMCO4 |
| 770 | SYN2, NSMF, TAP1, ARHGEF35, RAPGEF3 |
| 771 | SYN2, NSMF, TAP1, ARHGEF35, NPC2 |
| 772 | SYN2, NSMF, TAP1, ARHGEF16, MICB |
| 773 | SYN2, NSMF, TAP1, ARHGEF16, TMCO4 |
| 774 | SYN2, NSMF, TAP1, ARHGEF16, RAPGEF3 |
| 775 | SYN2, NSMF, TAP1, ARHGEF16, NPC2 |
| 776 | SYN2, NSMF, TAP1, MICB, TMCO4 |
| 777 | SYN2, NSMF, TAP1, MICB, RAPGEF3 |
| 778 | SYN2, NSMF, TAP1, MICB, NPC2 |
| 779 | SYN2, NSMF, TAP1, TMCO4, RAPGEF3 |
| 780 | SYN2, NSMF, TAP1, TMCO4, NPC2 |
| 781 | SYN2, NSMF, TAP1, RAPGEF3, NPC2 |
| 782 | SYN2, NSMF, PLCG2, ARHGEF35, ARHGEF16 |
| 783 | SYN2, NSMF, PLCG2, ARHGEF35, MICB |
| 784 | SYN2, NSMF, PLCG2, ARHGEF35, TMCO4 |
| 785 | SYN2, NSMF, PLCG2, ARHGEF35, RAPGEF3 |
| 786 | SYN2, NSMF, PLCG2, ARHGEF35, NPC2 |
| 787 | SYN2, NSMF, PLCG2, ARHGEF16, MICB |
| 788 | SYN2, NSMF, PLCG2, ARHGEF16, TMCO4 |
| 789 | SYN2, NSMF, PLCG2, ARHGEF16, RAPGEF3 |
| 790 | SYN2, NSMF, PLCG2, ARHGEF16, NPC2 |
| 791 | SYN2, NSMF, PLCG2, MICB, TMCO4 |
| 792 | SYN2, NSMF, PLCG2, MICB, RAPGEF3 |
| 793 | SYN2, NSMF, PLCG2, MICB, NPC2 |
| 794 | SYN2, NSMF, PLCG2, TMCO4, RAPGEF3 |
| 795 | SYN2, NSMF, PLCG2, TMCO4, NPC2 |
| 796 | SYN2, NSMF, PLCG2, RAPGEF3, NPC2 |
| 797 | SYN2, NSMF, ARHGEF35, ARHGEF16, MICB |
| 798 | SYN2, NSMF, ARHGEF35, ARHGEF16, TMCO4 |
| 799 | SYN2, NSMF, ARHGEF35, ARHGEF16, RAPGEF3 |
| 800 | SYN2, NSMF, ARHGEF35, ARHGEF16, NPC2 |
| 801 | SYN2, NSMF, ARHGEF35, MICB, TMCO4 |
| 802 | SYN2, NSMF, ARHGEF35, MICB, RAPGEF3 |
| 803 | SYN2, NSMF, ARHGEF35, MICB, NPC2 |
| 804 | SYN2, NSMF, ARHGEF35, TMCO4, RAPGEF3 |
| 805 | SYN2, NSMF, ARHGEF35, TMCO4, NPC2 |
| 806 | SYN2, NSMF, ARHGEF35, RAPGEF3, NPC2 |
| 807 | SYN2, NSMF, ARHGEF16, MICB, TMCO4 |
| 808 | SYN2, NSMF, ARHGEF16, MICB, RAPGEF3 |
| 809 | SYN2, NSMF, ARHGEF16, MICB, NPC2 |
| 810 | SYN2, NSMF, ARHGEF16, TMCO4, RAPGEF3 |
| 811 | SYN2, NSMF, ARHGEF16, TMCO4, NPC2 |
| 812 | SYN2, NSMF, ARHGEF16, RAPGEF3, NPC2 |
| 813 | SYN2, NSMF, MICB, TMCO4, RAPGEF3 |
| 814 | SYN2, NSMF, MICB, TMCO4, NPC2 |
| 815 | SYN2, NSMF, MICB, RAPGEF3, NPC2 |
| 816 | SYN2, NSMF, TMCO4, RAPGEF3, NPC2 |
| 817 | SYN2, TAP1, PLCG2, ARHGEF35, ARHGEF16 |
| 818 | SYN2, TAP1, PLCG2, ARHGEF35, MICB |
| 819 | SYN2, TAP1, PLCG2, ARHGEF35, TMCO4 |
| 820 | SYN2, TAP1, PLCG2, ARHGEF35, RAPGEF3 |
| 821 | SYN2, TAP1, PLCG2, ARHGEF35, NPC2 |
| 822 | SYN2, TAP1, PLCG2, ARHGEF16, MICB |
| 823 | SYN2, TAP1, PLCG2, ARHGEF16, TMCO4 |
| 824 | SYN2, TAP1, PLCG2, ARHGEF16, RAPGEF3 |
| 825 | SYN2, TAP1, PLCG2, ARHGEF16, NPC2 |
| 826 | SYN2, TAP1, PLCG2, MICB, TMCO4 |
| 827 | SYN2, TAP1, PLCG2, MICB, RAPGEF3 |
| 828 | SYN2, TAP1, PLCG2, MICB, NPC2 |
| 829 | SYN2, TAP1, PLCG2, TMCO4, RAPGEF3 |
| 830 | SYN2, TAP1, PLCG2, TMCO4, NPC2 |
| 831 | SYN2, TAP1, PLCG2, RAPGEF3, NPC2 |
| 832 | SYN2, TAP1, ARHGEF35, ARHGEF16, MICB |
| 833 | SYN2, TAP1, ARHGEF35, ARHGEF16, TMCO4 |
| 834 | SYN2, TAP1, ARHGEF35, ARHGEF16, RAPGEF3 |
| 835 | SYN2, TAP1, ARHGEF35, ARHGEF16, NPC2 |
| 836 | SYN2, TAP1, ARHGEF35, MICB, TMCO4 |
| 837 | SYN2, TAP1, ARHGEF35, MICB, RAPGEF3 |
| 838 | SYN2, TAP1, ARHGEF35, MICB, NPC2 |
| 839 | SYN2, TAP1, ARHGEF35, TMCO4, RAPGEF3 |
| 840 | SYN2, TAP1, ARHGEF35, TMCO4, NPC2 |
| 841 | SYN2, TAP1, ARHGEF35, RAPGEF3, NPC2 |
| 842 | SYN2, TAP1, ARHGEF16, MICB, TMCO4 |
| 843 | SYN2, TAP1, ARHGEF16, MICB, RAPGEF3 |
| 844 | SYN2, TAP1, ARHGEF16, MICB, NPC2 |
| 845 | SYN2, TAP1, ARHGEF16, TMCO4, RAPGEF3 |
| 846 | SYN2, TAP1, ARHGEF16, TMCO4, NPC2 |
| 847 | SYN2, TAP1, ARHGEF16, RAPGEF3, NPC2 |
| 848 | SYN2, TAP1, MICB, TMCO4, RAPGEF3 |
| 849 | SYN2, TAP1, MICB, TMCO4, NPC2 |
| 850 | SYN2, TAP1, MICB, RAPGEF3, NPC2 |
| 851 | SYN2, TAP1, TMCO4, RAPGEF3, NPC2 |
| 852 | SYN2, PLCG2, ARHGEF35, ARHGEF16, MICB |
| 853 | SYN2, PLCG2, ARHGEF35, ARHGEF16, TMCO4 |
| 854 | SYN2, PLCG2, ARHGEF35, ARHGEF16, RAPGEF3 |
| 855 | SYN2, PLCG2, ARHGEF35, ARHGEF16, NPC2 |
| 856 | SYN2, PLCG2, ARHGEF35, MICB, TMCO4 |
| 857 | SYN2, PLCG2, ARHGEF35, MICB, RAPGEF3 |
| 858 | SYN2, PLCG2, ARHGEF35, MICB, NPC2 |
| 859 | SYN2, PLCG2, ARHGEF35, TMCO4, RAPGEF3 |
| 860 | SYN2, PLCG2, ARHGEF35, TMCO4, NPC2 |
| 861 | SYN2, PLCG2, ARHGEF35, RAPGEF3, NPC2 |
| 862 | SYN2, PLCG2, ARHGEF16, MICB, TMCO4 |
| 863 | SYN2, PLCG2, ARHGEF16, MICB, RAPGEF3 |
| 864 | SYN2, PLCG2, ARHGEF16, MICB, NPC2 |
| 865 | SYN2, PLCG2, ARHGEF16, TMCO4, RAPGEF3 |
| 866 | SYN2, PLCG2, ARHGEF16, TMCO4, NPC2 |
| 867 | SYN2, PLCG2, ARHGEF16, RAPGEF3, NPC2 |
| 868 | SYN2, PLCG2, MICB, TMCO4, RAPGEF3 |
| 869 | SYN2, PLCG2, MICB, TMCO4, NPC2 |
| 870 | SYN2, PLCG2, MICB, RAPGEF3, NPC2 |
| 871 | SYN2, PLCG2, TMCO4, RAPGEF3, NPC2 |
| 872 | SYN2, ARHGEF35, ARHGEF16, MICB, TMCO4 |
| 873 | SYN2, ARHGEF35, ARHGEF16, MICB, RAPGEF3 |
| 874 | SYN2, ARHGEF35, ARHGEF16, MICB, NPC2 |
| 875 | SYN2, ARHGEF35, ARHGEF16, TMCO4, RAPGEF3 |
| 876 | SYN2, ARHGEF35, ARHGEF16, TMCO4, NPC2 |
| 877 | SYN2, ARHGEF35, ARHGEF16, RAPGEF3, NPC2 |
| 878 | SYN2, ARHGEF35, MICB, TMCO4, RAPGEF3 |
| 879 | SYN2, ARHGEF35, MICB, TMCO4, NPC2 |
| 880 | SYN2, ARHGEF35, MICB, RAPGEF3, NPC2 |
| 881 | SYN2, ARHGEF35, TMCO4, RAPGEF3, NPC2 |
| 882 | SYN2, ARHGEF16, MICB, TMCO4, RAPGEF3 |
| 883 | SYN2, ARHGEF16, MICB, TMCO4, NPC2 |
| 884 | SYN2, ARHGEF16, MICB, RAPGEF3, NPC2 |
| 885 | SYN2, ARHGEF16, TMCO4, RAPGEF3, NPC2 |
| 886 | SYN2, MICB, TMCO4, RAPGEF3, NPC2 |
| 887 | NSMF, TAP1, PLCG2, ARHGEF35, ARHGEF16 |
| 888 | NSMF, TAP1, PLCG2, ARHGEF35, MICB |
| 889 | NSMF, TAP1, PLCG2, ARHGEF35, TMCO4 |
| 890 | NSMF, TAP1, PLCG2, ARHGEF35, RAPGEF3 |
| 891 | NSMF, TAP1, PLCG2, ARHGEF35, NPC2 |
| 892 | NSMF, TAP1, PLCG2, ARHGEF16, MICB |
| 893 | NSMF, TAP1, PLCG2, ARHGEF16, TMCO4 |
| 894 | NSMF, TAP1, PLCG2, ARHGEF16, RAPGEF3 |
| 895 | NSMF, TAP1, PLCG2, ARHGEF16, NPC2 |
| 896 | NSMF, TAP1, PLCG2, MICB, TMCO4 |
| 897 | NSMF, TAP1, PLCG2, MICB, RAPGEF3 |
| 898 | NSMF, TAP1, PLCG2, MICB, NPC2 |
| 899 | NSMF, TAP1, PLCG2, TMCO4, RAPGEF3 |
| 900 | NSMF, TAP1, PLCG2, TMCO4, NPC2 |
| 901 | NSMF, TAP1, PLCG2, RAPGEF3, NPC2 |
| 902 | NSMF, TAP1, ARHGEF35, ARHGEF16, MICB |
| 903 | NSMF, TAP1, ARHGEF35, ARHGEF16, TMCO4 |
| 904 | NSMF, TAP1, ARHGEF35, ARHGEF16, RAPGEF3 |
| 905 | NSMF, TAP1, ARHGEF35, ARHGEF16, NPC2 |
| 906 | NSMF, TAP1, ARHGEF35, MICB, TMCO4 |
| 907 | NSMF, TAP1, ARHGEF35, MICB, RAPGEF3 |
| 908 | NSMF, TAP1, ARHGEF35, MICB, NPC2 |
| 909 | NSMF, TAP1, ARHGEF35, TMCO4, RAPGEF3 |
| 910 | NSMF, TAP1, ARHGEF35, TMCO4, NPC2 |
| 911 | NSMF, TAP1, ARHGEF35, RAPGEF3, NPC2 |
| 912 | NSMF, TAP1, ARHGEF16, MICB, TMCO4 |
| 913 | NSMF, TAP1, ARHGEF16, MICB, RAPGEF3 |
| 914 | NSMF, TAP1, ARHGEF16, MICB, NPC2 |
| 915 | NSMF, TAP1, ARHGEF16, TMCO4, RAPGEF3 |
| 916 | NSMF, TAP1, ARHGEF16, TMCO4, NPC2 |
| 917 | NSMF, TAP1, ARHGEF16, RAPGEF3, NPC2 |
| 918 | NSMF, TAP1, MICB, TMCO4, RAPGEF3 |
| 919 | NSMF, TAP1, MICB, TMCO4, NPC2 |
| 920 | NSMF, TAP1, MICB, RAPGEF3, NPC2 |
| 921 | NSMF, TAP1, TMCO4, RAPGEF3, NPC2 |
| 922 | NSMF, PLCG2, ARHGEF35, ARHGEF16, MICB |
| 923 | NSMF, PLCG2, ARHGEF35, ARHGEF16, TMCO4 |
| 924 | NSMF, PLCG2, ARHGEF35, ARHGEF16, RAPGEF3 |
| 925 | NSMF, PLCG2, ARHGEF35, ARHGEF16, NPC2 |
| 926 | NSMF, PLCG2, ARHGEF35, MICB, TMCO4 |
| 927 | NSMF, PLCG2, ARHGEF35, MICB, RAPGEF3 |
| 928 | NSMF, PLCG2, ARHGEF35, MICB, NPC2 |
| 929 | NSMF, PLCG2, ARHGEF35, TMCO4, RAPGEF3 |
| 930 | NSMF, PLCG2, ARHGEF35, TMCO4, NPC2 |
| 931 | NSMF, PLCG2, ARHGEF35, RAPGEF3, NPC2 |
| 932 | NSMF, PLCG2, ARHGEF16, MICB, TMCO4 |
| 933 | NSMF, PLCG2, ARHGEF16, MICB, RAPGEF3 |
| 934 | NSMF, PLCG2, ARHGEF16, MICB, NPC2 |
| 935 | NSMF, PLCG2, ARHGEF16, TMCO4, RAPGEF3 |
| 936 | NSMF, PLCG2, ARHGEF16, TMCO4, NPC2 |
| 937 | NSMF, PLCG2, ARHGEF16, RAPGEF3, NPC2 |
| 938 | NSMF, PLCG2, MICB, TMCO4, RAPGEF3 |
| 939 | NSMF, PLCG2, MICB, TMCO4, NPC2 |
| 940 | NSMF, PLCG2, MICB, RAPGEF3, NPC2 |
| 941 | NSMF, PLCG2, TMCO4, RAPGEF3, NPC2 |
| 942 | NSMF, ARHGEF35, ARHGEF16, MICB, TMCO4 |
| 943 | NSMF, ARHGEF35, ARHGEF16, MICB, RAPGEF3 |
| 944 | NSMF, ARHGEF35, ARHGEF16, MICB, NPC2 |
| 945 | NSMF, ARHGEF35, ARHGEF16, TMCO4, RAPGEF3 |
| 946 | NSMF, ARHGEF35, ARHGEF16, TMCO4, NPC2 |
| 947 | NSMF, ARHGEF35, ARHGEF16, RAPGEF3, NPC2 |
| 948 | NSMF, ARHGEF35, MICB, TMCO4, RAPGEF3 |
| 949 | NSMF, ARHGEF35, MICB, TMCO4, NPC2 |
| 950 | NSMF, ARHGEF35, MICB, RAPGEF3, NPC2 |
| 951 | NSMF, ARHGEF35, TMCO4, RAPGEF3, NPC2 |
| 952 | NSMF, ARHGEF16, MICB, TMCO4, RAPGEF3 |
| 953 | NSMF, ARHGEF16, MICB, TMCO4, NPC2 |
| 954 | NSMF, ARHGEF16, MICB, RAPGEF3, NPC2 |
| 955 | NSMF, ARHGEF16, TMCO4, RAPGEF3, NPC2 |
| 956 | NSMF, MICB, TMCO4, RAPGEF3, NPC2 |
| 957 | TAP1, PLCG2, ARHGEF35, ARHGEF16, MICB |
| 958 | TAP1, PLCG2, ARHGEF35, ARHGEF16, TMCO4 |
| 959 | TAP1, PLCG2, ARHGEF35, ARHGEF16, RAPGEF3 |
| 960 | TAP1, PLCG2, ARHGEF35, ARHGEF16, NPC2 |
| 961 | TAP1, PLCG2, ARHGEF35, MICB, TMCO4 |
| 962 | TAP1, PLCG2, ARHGEF35, MICB, RAPGEF3 |
| 963 | TAP1, PLCG2, ARHGEF35, MICB, NPC2 |
| 964 | TAP1, PLCG2, ARHGEF35, TMCO4, RAPGEF3 |
| 965 | TAP1, PLCG2, ARHGEF35, TMCO4, NPC2 |
| 966 | TAP1, PLCG2, ARHGEF35, RAPGEF3, NPC2 |
| 967 | TAP1, PLCG2, ARHGEF16, MICB, TMCO4 |
| 968 | TAP1, PLCG2, ARHGEF16, MICB, RAPGEF3 |
| 969 | TAP1, PLCG2, ARHGEF16, MICB, NPC2 |
| 970 | TAP1, PLCG2, ARHGEF16, TMCO4, RAPGEF3 |
| 971 | TAP1, PLCG2, ARHGEF16, TMCO4, NPC2 |
| 972 | TAP1, PLCG2, ARHGEF16, RAPGEF3, NPC2 |
| 973 | TAP1, PLCG2, MICB, TMCO4, RAPGEF3 |
| 974 | TAP1, PLCG2, MICB, TMCO4, NPC2 |
| 975 | TAP1, PLCG2, MICB, RAPGEF3, NPC2 |
| 976 | TAP1, PLCG2, TMCO4, RAPGEF3, NPC2 |
| 977 | TAP1, ARHGEF35, ARHGEF16, MICB, TMCO4 |
| 978 | TAP1, ARHGEF35, ARHGEF16, MICB, RAPGEF3 |
| 979 | TAP1, ARHGEF35, ARHGEF16, MICB, NPC2 |
| 980 | TAP1, ARHGEF35, ARHGEF16, TMCO4, RAPGEF3 |
| 981 | TAP1, ARHGEF35, ARHGEF16, TMCO4, NPC2 |
| 982 | TAP1, ARHGEF35, ARHGEF16, RAPGEF3, NPC2 |
| 983 | TAP1, ARHGEF35, MICB, TMCO4, RAPGEF3 |
| 984 | TAP1, ARHGEF35, MICB, TMCO4, NPC2 |
| 985 | TAP1, ARHGEF35, MICB, RAPGEF3, NPC2 |
| 986 | TAP1, ARHGEF35, TMCO4, RAPGEF3, NPC2 |
| 987 | TAP1, ARHGEF16, MICB, TMCO4, RAPGEF3 |
| 988 | TAP1, ARHGEF16, MICB, TMCO4, NPC2 |
| 989 | TAP1, ARHGEF16, MICB, RAPGEF3, NPC2 |
| 990 | TAP1, ARHGEF16, TMCO4, RAPGEF3, NPC2 |
| 991 | TAP1, MICB, TMCO4, RAPGEF3, NPC2 |
| 992 | PLCG2, ARHGEF35, ARHGEF16, MICB, TMCO4 |
| 993 | PLCG2, ARHGEF35, ARHGEF16, MICB, RAPGEF3 |
| 994 | PLCG2, ARHGEF35, ARHGEF16, MICB, NPC2 |
| 995 | PLCG2, ARHGEF35, ARHGEF16, TMCO4, RAPGEF3 |
| 996 | PLCG2, ARHGEF35, ARHGEF16, TMCO4, NPC2 |
| 997 | PLCG2, ARHGEF35, ARHGEF16, RAPGEF3, NPC2 |
| 998 | PLCG2, ARHGEF35, MICB, TMCO4, RAPGEF3 |
| 999 | PLCG2, ARHGEF35, MICB, TMCO4, NPC2 |
| 1000 | PLCG2, ARHGEF35, MICB, RAPGEF3, NPC2 |
| 1001 | PLCG2, ARHGEF35, TMCO4, RAPGEF3, NPC2 |
| 1002 | PLCG2, ARHGEF16, MICB, TMCO4, RAPGEF3 |
| 1003 | PLCG2, ARHGEF16, MICB, TMCO4, NPC2 |
| 1004 | PLCG2, ARHGEF16, MICB, RAPGEF3, NPC2 |
| 1005 | PLCG2, ARHGEF16, TMCO4, RAPGEF3, NPC2 |
| 1006 | PLCG2, MICB, TMCO4, RAPGEF3, NPC2 |
| 1007 | ARHGEF35, ARHGEF16, MICB, TMCO4, RAPGEF3 |
| 1008 | ARHGEF35, ARHGEF16, MICB, TMCO4, NPC2 |
| 1009 | ARHGEF35, ARHGEF16, MICB, RAPGEF3, NPC2 |
| 1010 | ARHGEF35, ARHGEF16, TMCO4, RAPGEF3, NPC2 |
| 1011 | ARHGEF35, MICB, TMCO4, RAPGEF3, NPC2 |
| 1012 | ARHGEF16, MICB, TMCO4, RAPGEF3, NPC2 |

In some embodiments, the one or more genes comprise at least one gene selected from the group consisting of HLA-C, STAT6, TMCO4, CNRIP1, DCAF13, PRDM8, DACH1, and IKBKE. In some embodiments, the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE. In some embodiments, the one or more genes do not comprise ANTXR1. In some embodiments, the one or more genes do not comprise IFI35.

In some embodiments, the one or more genes comprise at least one gene selected from the group consisting of HLA-C, STAT6, TMCO4, and CNRIP1. In some embodiments, the one or more genes comprise at least 2 genes selected from the group consisting of HLA-C, STAT6, TMCO4, and CNRIP1. In some embodiments, the one or more genes comprise at least 3 genes selected from the group consisting of HLA-C, STAT6, TMCO4, and CNRIP1. In some embodiments, the one or more genes comprise all of HLA-C, STAT6, TMCO4, and CNRIP1. In some embodiments, the one or more genes do not comprise ANTXR1. In some embodiments, the one or more genes do not comprise IFI35.

In some embodiments, the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7. In some embodiments, the one or more genes comprise at least 2, 3, 4, 5, 6, 7, 8, 9, 10, 11, 12, 13, 14, 15, 16, 17, 18, 19, 20, or 21 genes selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7. In some embodiments, the one or more genes do not comprise ANTXR1. In some embodiments, the one or more genes do not comprise IFI35.

In some embodiments, the one or more genes comprise at least one gene selected from the group consisting of HLA-C, CNRIP1, JPH1, TMCO4, STAT6, DENND2D, and ETV7. In some embodiments, the one or more genes comprise at least 2, 3, 4, 5, 6, or 7 genes selected from the group consisting of HLA-C, CNRIP1, JPH1, TMCO4, STAT6, DENND2D, and ETV7. In some embodiments, the one or more genes do not comprise ANTXR1. In some embodiments, the one or more genes do not comprise IFI35.

In some embodiments, the one or more genes comprise at least one gene selected from the group consisting of HLA-C, CNRIP1, JPH1, and TMCO4. In some embodiments, the one or more genes comprise HLA-C. In some embodiments, the one or more genes comprise at least 2 genes selected from the group consisting of HLA-C, CNRIP1, JPH1, and TMCO4. In some embodiments, the one or more genes comprise at least 3 genes selected from the group consisting of HLA-C, CNRIP1, JPH1, and TMCO4. In some embodiments, the one or more genes comprise all of HLA-C, CNRIP1, JPH1, and TMCO4. In some embodiments, the one or more genes do not comprise ANTXR1. In some embodiments, the one or more genes do not comprise IFI35.

In some embodiments, the one or more genes comprise one of the gene combinations selected from Table 14 below. In some embodiments, the one or more genes comprise two genes selected from combinations #1 to #55 of Table 14 below. In some embodiments, the one or more genes comprise three genes selected from combinations #56 to #220 of Table 14 below. In some embodiments, the one or more genes comprise four genes selected from combinations #221 to #550 of Table 14 below. In some embodiments, the one or more genes comprise five genes selected from combinations #551 to #1012 of Table 14 below. In some embodiments, the one or more genes do not comprise ANTXR1. In some embodiments, the one or more genes do not comprise IFI35.

TABLE 14
Non-limiting Examples of Gene Combination (C#)

| C# | Genes |
|---|---|
| 1 | HLA-C, CNRIP1 |
| 2 | HLA-C, JPH1 |
| 3 | HLA-C, TMCO4 |
| 4 | HLA-C, STAT6 |
| 5 | HLA-C, DENND2D |
| 6 | HLA-C, ETV7 |
| 7 | HLA-C, DCAF13 |
| 8 | HLA-C, PRDM8 |
| 9 | HLA-C, DACH1 |
| 10 | HLA-C, IKBKE |
| 11 | CNRIP1, JPH1 |
| 12 | CNRIP1, TMCO4 |
| 13 | CNRIP1, STAT6 |
| 14 | CNRIP1, DENND2D |
| 15 | CNRIP1, ETV7 |
| 16 | CNRIP1, DCAF13 |
| 17 | CNRIP1, PRDM8 |
| 18 | CNRIP1, DACH1 |
| 19 | CNRIP1, IKBKE |
| 20 | JPH1, TMCO4 |
| 21 | JPH1, STAT6 |
| 22 | JPH1, DENND2D |
| 23 | JPH1, ETV7 |
| 24 | JPH1, DCAF13 |
| 25 | JPH1, PRDM8 |
| 26 | JPH1, DACH1 |
| 27 | JPH1, IKBKE |
| 28 | TMCO4, STAT6 |
| 29 | TMCO4, DENND2D |
| 30 | TMCO4, ETV7 |
| 31 | TMCO4, DCAF13 |
| 32 | TMCO4, PRDM8 |
| 33 | TMCO4, DACH1 |
| 34 | TMCO4, IKBKE |
| 35 | STAT6, DENND2D |
| 36 | STAT6, ETV7 |
| 37 | STAT6, DCAF13 |
| 38 | STAT6, PRDM8 |
| 39 | STAT6, DACH1 |
| 40 | STAT6, IKBKE |
| 41 | DENND2D, ETV7 |
| 42 | DENND2D, DCAF13 |
| 43 | DENND2D, PRDM8 |
| 44 | DENND2D, DACH1 |
| 45 | DENND2D, IKBKE |
| 46 | ETV7, DCAF13 |
| 47 | ETV7, PRDM8 |
| 48 | ETV7, DACH1 |
| 49 | ETV7, IKBKE |
| 50 | DCAF13, PRDM8 |
| 51 | DCAF13, DACH1 |
| 52 | DCAF13, IKBKE |
| 53 | PRDM8, DACH1 |
| 54 | PRDM8, IKBKE |
| 55 | DACH1, IKBKE |
| 56 | HLA-C, CNRIP1, JPH1 |
| 57 | HLA-C, CNRIP1, TMCO4 |
| 58 | HLA-C, CNRIP1, STAT6 |
| 59 | HLA-C, CNRIP1, DENND2D |
| 60 | HLA-C, CNRIP1, ETV7 |
| 61 | HLA-C, CNRIP1, DCAF13 |
| 62 | HLA-C, CNRIP1, PRDM8 |
| 63 | HLA-C, CNRIP1, DACH1 |
| 64 | HLA-C, CNRIP1, IKBKE |
| 65 | HLA-C, JPH1, TMCO4 |
| 66 | HLA-C, JPH1, STAT6 |
| 67 | HLA-C, JPH1, DENND2D |
| 68 | HLA-C, JPH1, ETV7 |
| 69 | HLA-C, JPH1, DCAF13 |
| 70 | HLA-C, JPH1, PRDM8 |
| 71 | HLA-C, JPH1, DACH1 |
| 72 | HLA-C, JPH1, IKBKE |
| 73 | HLA-C, TMCO4, STAT6 |
| 74 | HLA-C, TMCO4, DENND2D |
| 75 | HLA-C, TMCO4, ETV7 |
| 76 | HLA-C, TMCO4, DCAF13 |
| 77 | HLA-C, TMCO4, PRDM8 |
| 78 | HLA-C, TMCO4, DACH1 |
| 79 | HLA-C, TMCO4, IKBKE |
| 80 | HLA-C, STAT6, DENND2D |
| 81 | HLA-C, STAT6, ETV7 |
| 82 | HLA-C, STAT6, DCAF13 |
| 83 | HLA-C, STAT6, PRDM8 |
| 84 | HLA-C, STAT6, DACH1 |
| 85 | HLA-C, STAT6, IKBKE |
| 86 | HLA-C, DENND2D, ETV7 |
| 87 | HLA-C, DENND2D, DCAF13 |
| 88 | HLA-C, DENND2D, PRDM8 |
| 89 | HLA-C, DENND2D, DACH1 |
| 90 | HLA-C, DENND2D, IKBKE |
| 91 | HLA-C, ETV7, DCAF13 |
| 92 | HLA-C, ETV7, PRDM8 |
| 93 | HLA-C, ETV7, DACH1 |
| 94 | HLA-C, ETV7, IKBKE |
| 95 | HLA-C, DCAF13, PRDM8 |
| 96 | HLA-C, DCAF13, DACH1 |
| 97 | HLA-C, DCAF13, IKBKE |
| 98 | HLA-C, PRDM8, DACH1 |
| 99 | HLA-C, PRDM8, IKBKE |
| 100 | HLA-C, DACH1, IKBKE |
| 101 | CNRIP1, JPH1, TMCO4 |
| 102 | CNRIP1, JPH1, STAT6 |
| 103 | CNRIP1, JPH1, DENND2D |
| 104 | CNRIP1, JPH1, ETV7 |
| 105 | CNRIP1, JPH1, DCAF13 |
| 106 | CNRIP1, JPH1, PRDM8 |
| 107 | CNRIP1, JPH1, DACH1 |
| 108 | CNRIP1, JPH1, IKBKE |
| 109 | CNRIP1, TMCO4, STAT6 |
| 110 | CNRIP1, TMCO4, DENND2D |
| 111 | CNRIP1, TMCO4, ETV7 |
| 112 | CNRIP1, TMCO4, DCAF13 |
| 113 | CNRIP1, TMCO4, PRDM8 |
| 114 | CNRIP1, TMCO4, DACH1 |
| 115 | CNRIP1, TMCO4, IKBKE |
| 116 | CNRIP1, STAT6, DENND2D |
| 117 | CNRIP1, STAT6, ETV7 |
| 118 | CNRIP1, STAT6, DCAF13 |
| 119 | CNRIP1, STAT6, PRDM8 |
| 120 | CNRIP1, STAT6, DACH1 |
| 121 | CNRIP1, STAT6, IKBKE |
| 122 | CNRIP1, DENND2D, ETV7 |
| 123 | CNRIP1, DENND2D, DCAF13 |
| 124 | CNRIP1, DENND2D, PRDM8 |
| 125 | CNRIP1, DENND2D, DACH1 |
| 126 | CNRIP1, DENND2D, IKBKE |
| 127 | CNRIP1, ETV7, DCAF13 |
| 128 | CNRIP1, ETV7, PRDM8 |
| 129 | CNRIP1, ETV7, DACH1 |
| 130 | CNRIP1, ETV7, IKBKE |
| 131 | CNRIP1, DCAF13, PRDM8 |
| 132 | CNRIP1, DCAF13, DACH1 |
| 133 | CNRIP1, DCAF13, IKBKE |
| 134 | CNRIP1, PRDM8, DACH1 |
| 135 | CNRIP1, PRDM8, IKBKE |
| 136 | CNRIP1, DACH1, IKBKE |
| 137 | JPH1, TMCO4, STAT6 |
| 138 | JPH1, TMCO4, DENND2D |
| 139 | JPH1, TMCO4, ETV7 |
| 140 | JPH1, TMCO4, DCAF13 |
| 141 | JPH1, TMCO4, PRDM8 |
| 142 | JPH1, TMCO4, DACH1 |
| 143 | JPH1, TMCO4, IKBKE |
| 144 | JPH1, STAT6, DENND2D |
| 145 | JPH1, STAT6, ETV7 |
| 146 | JPH1, STAT6, DCAF13 |
| 147 | JPH1, STAT6, PRDM8 |
| 148 | JPH1, STAT6, DACH1 |
| 149 | JPH1, STAT6, IKBKE |
| 150 | JPH1, DENND2D, ETV7 |
| 151 | JPH1, DENND2D, DCAF13 |
| 152 | JPH1, DENND2D, PRDM8 |
| 153 | JPH1, DENND2D, DACH1 |
| 154 | JPH1, DENND2D, IKBKE |
| 155 | JPH1, ETV7, DCAF13 |
| 156 | JPH1, ETV7, PRDM8 |
| 157 | JPH1, ETV7, DACH1 |
| 158 | JPH1, ETV7, IKBKE |
| 159 | JPH1, DCAF13, PRDM8 |
| 160 | JPH1, DCAF13, DACH1 |
| 161 | JPH1, DCAF13, IKBKE |
| 162 | JPH1, PRDM8, DACH1 |
| 163 | JPH1, PRDM8, IKBKE |
| 164 | JPH1, DACH1, IKBKE |
| 165 | TMCO4, STAT6, DENND2D |
| 166 | TMCO4, STAT6, ETV7 |
| 167 | TMCO4, STAT6, DCAF13 |
| 168 | TMCO4, STAT6, PRDM8 |
| 169 | TMCO4, STAT6, DACH1 |
| 170 | TMCO4, STAT6, IKBKE |
| 171 | TMCO4, DENND2D, ETV7 |
| 172 | TMCO4, DENND2D, DCAF13 |
| 173 | TMCO4, DENND2D, PRDM8 |
| 174 | TMCO4, DENND2D, DACH1 |
| 175 | TMCO4, DENND2D, IKBKE |
| 176 | TMCO4, ETV7, DCAF13 |
| 177 | TMCO4, ETV7, PRDM8 |
| 178 | TMCO4, ETV7, DACH1 |
| 179 | TMCO4, ETV7, IKBKE |
| 180 | TMCO4, DCAF13, PRDM8 |
| 181 | TMCO4, DCAF13, DACH1 |
| 182 | TMCO4, DCAF13, IKBKE |
| 183 | TMCO4, PRDM8, DACH1 |
| 184 | TMCO4, PRDM8, IKBKE |
| 185 | TMCO4, DACH1, IKBKE |
| 186 | STAT6, DENND2D, ETV7 |
| 187 | STAT6, DENND2D, DCAF13 |
| 188 | STAT6, DENND2D, PRDM8 |
| 189 | STAT6, DENND2D, DACH1 |
| 190 | STAT6, DENND2D, IKBKE |
| 191 | STAT6, ETV7, DCAF13 |
| 192 | STAT6, ETV7, PRDM8 |
| 193 | STAT6, ETV7, DACH1 |
| 194 | STAT6, ETV7, IKBKE |
| 195 | STAT6, DCAF13, PRDM8 |
| 196 | STAT6, DCAF13, DACH1 |
| 197 | STAT6, DCAF13, IKBKE |
| 198 | STAT6, PRDM8, DACH1 |
| 199 | STAT6, PRDM8, IKBKE |
| 200 | STAT6, DACH1, IKBKE |
| 201 | DENND2D, ETV7, DCAF13 |
| 202 | DENND2D, ETV7, PRDM8 |
| 203 | DENND2D, ETV7, DACH1 |
| 204 | DENND2D, ETV7, IKBKE |
| 205 | DENND2D, DCAF13, PRDM8 |
| 206 | DENND2D, DCAF13, DACH1 |
| 207 | DENND2D, DCAF13, IKBKE |
| 208 | DENND2D, PRDM8, DACH1 |
| 209 | DENND2D, PRDM8, IKBKE |
| 210 | DENND2D, DACH1, IKBKE |
| 211 | ETV7, DCAF13, PRDM8 |
| 212 | ETV7, DCAF13, DACH1 |
| 213 | ETV7, DCAF13, IKBKE |
| 214 | ETV7, PRDM8, DACH1 |
| 215 | ETV7, PRDM8, IKBKE |
| 216 | ETV7, DACH1, IKBKE |
| 217 | DCAF13, PRDM8, DACH1 |
| 218 | DCAF13, PRDM8, IKBKE |
| 219 | DCAF13, DACH1, IKBKE |
| 220 | PRDM8, DACH1, IKBKE |
| 221 | HLA-C, CNRIP1, JPH1, TMCO4 |
| 222 | HLA-C, CNRIP1, JPH1, STAT6 |
| 223 | HLA-C, CNRIP1, JPH1, DENND2D |
| 224 | HLA-C, CNRIP1, JPH1, ETV7 |
| 225 | HLA-C, CNRIP1, JPH1, DCAF13 |
| 226 | HLA-C, CNRIP1, JPH1, PRDM8 |
| 227 | HLA-C, CNRIP1, JPH1, DACH1 |
| 228 | HLA-C, CNRIP1, JPH1, IKBKE |
| 229 | HLA-C, CNRIP1, TMCO4, STAT6 |
| 230 | HLA-C, CNRIP1, TMCO4, DENND2D |
| 231 | HLA-C, CNRIP1, TMCO4, ETV7 |
| 232 | HLA-C, CNRIP1, TMCO4, DCAF13 |
| 233 | HLA-C, CNRIP1, TMCO4, PRDM8 |
| 234 | HLA-C, CNRIP1, TMCO4, DACH1 |
| 235 | HLA-C, CNRIP1, TMCO4, IKBKE |
| 236 | HLA-C, CNRIP1, STAT6, DENND2D |
| 237 | HLA-C, CNRIP1, STAT6, ETV7 |
| 238 | HLA-C, CNRIP1, STAT6, DCAF13 |
| 239 | HLA-C, CNRIP1, STAT6, PRDM8 |
| 240 | HLA-C, CNRIP1, STAT6, DACH1 |
| 241 | HLA-C, CNRIP1, STAT6, IKBKE |
| 242 | HLA-C, CNRIP1, DENND2D, ETV7 |
| 243 | HLA-C, CNRIP1, DENND2D, DCAF13 |
| 244 | HLA-C, CNRIP1, DENND2D, PRDM8 |
| 245 | HLA-C, CNRIP1, DENND2D, DACH1 |
| 246 | HLA-C, CNRIP1, DENND2D, IKBKE |
| 247 | HLA-C, CNRIP1, ETV7, DCAF13 |
| 248 | HLA-C, CNRIP1, ETV7, PRDM8 |
| 249 | HLA-C, CNRIP1, ETV7, DACH1 |
| 250 | HLA-C, CNRIP1, ETV7, IKBKE |
| 251 | HLA-C, CNRIP1, DCAF13, PRDM8 |
| 252 | HLA-C, CNRIP1, DCAF13, DACH1 |
| 253 | HLA-C, CNRIP1, DCAF13, IKBKE |
| 254 | HLA-C, CNRIP1, PRDM8, DACH1 |
| 255 | HLA-C, CNRIP1, PRDM8, IKBKE |
| 256 | HLA-C, CNRIP1, DACH1, IKBKE |
| 257 | HLA-C, JPH1, TMCO4, STAT6 |
| 258 | HLA-C, JPH1, TMCO4, DENND2D |
| 259 | HLA-C, JPH1, TMCO4, ETV7 |
| 260 | HLA-C, JPH1, TMCO4, DCAF13 |
| 261 | HLA-C, JPH1, TMCO4, PRDM8 |
| 262 | HLA-C, JPH1, TMCO4, DACH1 |
| 263 | HLA-C, JPH1, TMCO4, IKBKE |
| 264 | HLA-C, JPH1, STAT6, DENND2D |
| 265 | HLA-C, JPH1, STAT6, ETV7 |
| 266 | HLA-C, JPH1, STAT6, DCAF13 |
| 267 | HLA-C, JPH1, STAT6, PRDM8 |
| 268 | HLA-C, JPH1, STAT6, DACH1 |
| 269 | HLA-C, JPH1, STAT6, IKBKE |
| 270 | HLA-C, JPH1, DENND2D, ETV7 |
| 271 | HLA-C, JPH1, DENND2D, DCAF13 |
| 272 | HLA-C, JPH1, DENND2D, PRDM8 |
| 273 | HLA-C, JPH1, DENND2D, DACH1 |
| 274 | HLA-C, JPH1, DENND2D, IKBKE |
| 275 | HLA-C, JPH1, ETV7, DCAF13 |
| 276 | HLA-C, JPH1, ETV7, PRDM8 |
| 277 | HLA-C, JPH1, ETV7, DACH1 |
| 278 | HLA-C, JPH1, ETV7, IKBKE |
| 279 | HLA-C, JPH1, DCAF13, PRDM8 |
| 280 | HLA-C, JPH1, DCAF13, DACH1 |
| 281 | HLA-C, JPH1, DCAF13, IKBKE |
| 282 | HLA-C, JPH1, PRDM8, DACH1 |
| 283 | HLA-C, JPH1, PRDM8, IKBKE |
| 284 | HLA-C, JPH1, DACH1, IKBKE |
| 285 | HLA-C, TMCO4, STAT6, DENND2D |
| 286 | HLA-C, TMCO4, STAT6, ETV7 |
| 287 | HLA-C, TMCO4, STAT6, DCAF13 |
| 288 | HLA-C, TMCO4, STAT6, PRDM8 |
| 289 | HLA-C, TMCO4, STAT6, DACH1 |
| 290 | HLA-C, TMCO4, STAT6, IKBKE |
| 291 | HLA-C, TMCO4, DENND2D, ETV7 |
| 292 | HLA-C, TMCO4, DENND2D, DCAF13 |
| 293 | HLA-C, TMCO4, DENND2D, PRDM8 |
| 294 | HLA-C, TMCO4, DENND2D, DACH1 |
| 295 | HLA-C, TMCO4, DENND2D, IKBKE |
| 296 | HLA-C, TMCO4, ETV7, DCAF13 |
| 297 | HLA-C, TMCO4, ETV7, PRDM8 |
| 298 | HLA-C, TMCO4, ETV7, DACH1 |
| 299 | HLA-C, TMCO4, ETV7, IKBKE |
| 300 | HLA-C, TMCO4, DCAF13, PRDM8 |
| 301 | HLA-C, TMCO4, DCAF13, DACH1 |
| 302 | HLA-C, TMCO4, DCAF13, IKBKE |
| 303 | HLA-C, TMCO4, PRDM8, DACH1 |
| 304 | HLA-C, TMCO4, PRDM8, IKBKE |
| 305 | HLA-C, TMCO4, DACH1, IKBKE |
| 306 | HLA-C, STAT6, DENND2D, ETV7 |
| 307 | HLA-C, STAT6, DENND2D, DCAF13 |
| 308 | HLA-C, STAT6, DENND2D, PRDM8 |
| 309 | HLA-C, STAT6, DENND2D, DACH1 |
| 310 | HLA-C, STAT6, DENND2D, IKBKE |
| 311 | HLA-C, STAT6, ETV7, DCAF13 |
| 312 | HLA-C, STAT6, ETV7, PRDM8 |
| 313 | HLA-C, STAT6, ETV7, DACH1 |
| 314 | HLA-C, STAT6, ETV7, IKBKE |
| 315 | HLA-C, STAT6, DCAF13, PRDM8 |
| 316 | HLA-C, STAT6, DCAF13, DACH1 |
| 317 | HLA-C, STAT6, DCAF13, IKBKE |
| 318 | HLA-C, STAT6, PRDM8, DACH1 |
| 319 | HLA-C, STAT6, PRDM8, IKBKE |
| 320 | HLA-C, STAT6, DACH1, IKBKE |
| 321 | HLA-C, DENND2D, ETV7, DCAF13 |
| 322 | HLA-C, DENND2D, ETV7, PRDM8 |
| 323 | HLA-C, DENND2D, ETV7, DACH1 |
| 324 | HLA-C, DENND2D, ETV7, IKBKE |
| 325 | HLA-C, DENND2D, DCAF13, PRDM8 |
| 326 | HLA-C, DENND2D, DCAF13, DACH1 |
| 327 | HLA-C, DENND2D, DCAF13, IKBKE |
| 328 | HLA-C, DENND2D, PRDM8, DACH1 |
| 329 | HLA-C, DENND2D, PRDM8, IKBKE |
| 330 | HLA-C, DENND2D, DACH1, IKBKE |
| 331 | HLA-C, ETV7, DCAF13, PRDM8 |
| 332 | HLA-C, ETV7, DCAF13, DACH1 |
| 333 | HLA-C, ETV7, DCAF13, IKBKE |
| 334 | HLA-C, ETV7, PRDM8, DACH1 |
| 335 | HLA-C, ETV7, PRDM8, IKBKE |
| 336 | HLA-C, ETV7, DACH1, IKBKE |
| 337 | HLA-C, DCAF13, PRDM8, DACH1 |
| 338 | HLA-C, DCAF13, PRDM8, IKBKE |
| 339 | HLA-C, DCAF13, DACH1, IKBKE |
| 340 | HLA-C, PRDM8, DACH1, IKBKE |
| 341 | CNRIP1, JPH1, TMCO4, STAT6 |
| 342 | CNRIP1, JPH1, TMCO4, DENND2D |
| 343 | CNRIP1, JPH1, TMCO4, ETV7 |
| 344 | CNRIP1, JPH1, TMCO4, DCAF13 |
| 345 | CNRIP1, JPH1, TMCO4, PRDM8 |
| 346 | CNRIP1, JPH1, TMCO4, DACH1 |
| 347 | CNRIP1, JPH1, TMCO4, IKBKE |
| 348 | CNRIP1, JPH1, STAT6, DENND2D |
| 349 | CNRIP1, JPH1, STAT6, ETV7 |
| 350 | CNRIP1, JPH1, STAT6, DCAF13 |
| 351 | CNRIP1, JPH1, STAT6, PRDM8 |
| 352 | CNRIP1, JPH1, STAT6, DACH1 |
| 353 | CNRIP1, JPH1, STAT6, IKBKE |
| 354 | CNRIP1, JPH1, DENND2D, ETV7 |
| 355 | CNRIP1, JPH1, DENND2D, DCAF13 |
| 356 | CNRIP1, JPH1, DENND2D, PRDM8 |
| 357 | CNRIP1, JPH1, DENND2D, DACH1 |
| 358 | CNRIP1, JPH1, DENND2D, IKBKE |
| 359 | CNRIP1, JPH1, ETV7, DCAF13 |
| 360 | CNRIP1, JPH1, ETV7, PRDM8 |
| 361 | CNRIP1, JPH1, ETV7, DACH1 |
| 362 | CNRIP1, JPH1, ETV7, IKBKE |
| 363 | CNRIP1, JPH1, DCAF13, PRDM8 |
| 364 | CNRIP1, JPH1, DCAF13, DACH1 |
| 365 | CNRIP1, JPH1, DCAF13, IKBKE |
| 366 | CNRIP1, JPH1, PRDM8, DACH1 |
| 367 | CNRIP1, JPH1, PRDM8, IKBKE |
| 368 | CNRIP1, JPH1, DACH1, IKBKE |
| 369 | CNRIP1, TMCO4, STAT6, DENND2D |
| 370 | CNRIP1, TMCO4, STAT6, ETV7 |
| 371 | CNRIP1, TMCO4, STAT6, DCAF13 |
| 372 | CNRIP1, TMC04, STAT6, PRDM8 |
| 373 | CNRIP1, TMCO4, STAT6, DACH1 |
| 374 | CNRIP1, TMCO4, STAT6, IKBKE |
| 375 | CNRIP1, TMCO4, DENND2D, ETV7 |
| 376 | CNRIP1, TMCO4, DENND2D, DCAF13 |
| 377 | CNRIP1, TMCO4, DENND2D, PRDM8 |
| 378 | CNRIP1, TMCO4, DENND2D, DACH1 |
| 379 | CNRIP1, TMCO4, DENND2D, IKBKE |
| 380 | CNRIP1, TMCO4, ETV7, DCAF13 |
| 381 | CNRIP1, TMCO4, ETV7, PRDM8 |
| 382 | CNRIP1, TMCO4, ETV7, DACH1 |
| 383 | CNRIP1, TMCO4, ETV7, IKBKE |
| 384 | CNRIP1, TMCO4, DCAF13, PRDM8 |
| 385 | CNRIP1, TMCO4, DCAF13, DACH1 |
| 386 | CNRIP1, TMCO4, DCAF13, IKBKE |
| 387 | CNRIP1, TMCO4, PRDM8, DACH1 |
| 388 | CNRIP1, TMCO4, PRDM8, IKBKE |
| 389 | CNRIP1, TMCO4, DACH1, IKBKE |
| 390 | CNRIP1, STAT6, DENND2D, ETV7 |
| 391 | CNRIP1, STAT6, DENND2D, DCAF13 |
| 392 | CNRIP1, STAT6, DENND2D, PRDM8 |
| 393 | CNRIP1, STAT6, DENND2D, DACH1 |
| 394 | CNRIP1, STAT6, DENND2D, IKBKE |
| 395 | CNRIP1, STAT6, ETV7, DCAF13 |
| 396 | CNRIP1, STAT6, ETV7, PRDM8 |
| 397 | CNRIP1, STAT6, ETV7, DACH1 |
| 398 | CNRIP1, STAT6, ETV7, IKBKE |
| 399 | CNRIP1, STAT6, DCAF13, PRDM8 |
| 400 | CNRIP1, STAT6, DCAF13, DACH1 |
| 401 | CNRIP1, STAT6, DCAF13, IKBKE |
| 402 | CNRIP1, STAT6, PRDM8, DACH1 |
| 403 | CNRIP1, STAT6, PRDM8, IKBKE |
| 404 | CNRIP1, STAT6, DACH1, IKBKE |
| 405 | CNRIP1, DENND2D, ETV7, DCAF13 |
| 406 | CNRIP1, DENND2D, ETV7, PRDM8 |
| 407 | CNRIP1, DENND2D, ETV7, DACH1 |
| 408 | CNRIP1, DENND2D, ETV7, IKBKE |
| 409 | CNRIP1, DENND2D, DCAF13, PRDM8 |
| 410 | CNRIP1, DENND2D, DCAF13, DACH1 |
| 411 | CNRIP1, DENND2D, DCAF13, IKBKE |
| 412 | CNRIP1, DENND2D, PRDM8, DACH1 |
| 413 | CNRIP1, DENND2D, PRDM8, IKBKE |
| 414 | CNRIP1, DENND2D, DACH1, IKBKE |
| 415 | CNRIP1, ETV7, DCAF13, PRDM8 |
| 416 | CNRIP1, ETV7, DCAF13, DACH1 |
| 417 | CNRIP1, ETV7, DCAF13, IKBKE |
| 418 | CNRIP1, ETV7, PRDM8, DACH1 |
| 419 | CNRIP1, ETV7, PRDM8, IKBKE |
| 420 | CNRIP1, ETV7, DACH1, IKBKE |
| 421 | CNRIP1, DCAF13, PRDM8, DACH1 |
| 422 | CNRIP1, DCAF13, PRDM8, IKBKE |
| 423 | CNRIP1, DCAF13, DACH1, IKBKE |
| 424 | CNRIP1, PRDM8, DACH1, IKBKE |
| 425 | JPH1, TMCO4, STAT6, DENND2D |
| 426 | JPH1, TMCO4, STAT6, ETV7 |
| 427 | JPH1, TMCO4, STAT6, DCAF13 |
| 428 | JPH1, TMCO4, STAT6, PRDM8 |
| 429 | JPH1, TMCO4, STAT6, DACH1 |
| 430 | JPH1, TMCO4, STAT6, IKBKE |
| 431 | JPH1, TMCO4, DENND2D, ETV7 |
| 432 | JPH1, TMCO4, DENND2D, DCAF13 |
| 433 | JPH1, TMCO4, DENND2D, PRDM8 |
| 434 | JPH1, TMC04, DENND2D, DACH1 |
| 435 | JPH1, TMCO4, DENND2D, IKBKE |
| 436 | JPH1, TMCO4, ETV7, DCAF13 |
| 437 | JPH1, TMCO4, ETV7, PRDM8 |
| 438 | JPH1, TMCO4, ETV7, DACH1 |
| 439 | JPH1, TMCO4, ETV7, IKBKE |
| 440 | JPH1, TMCO4, DCAF13, PRDM8 |
| 441 | JPH1, TMCO4, DCAF13, DACH1 |
| 442 | JPH1, TMCO4, DCAF13, IKBKE |
| 443 | JPH1, TMCO4, PRDM8, DACH1 |
| 444 | JPH1, TMCO4, PRDM8, IKBKE |
| 445 | JPH1, TMCO4, DACH1, IKBKE |
| 446 | JPH1, STAT6, DENND2D, ETV7 |
| 447 | JPH1, STAT6, DENND2D, DCAF13 |
| 448 | JPH1, STAT6, DENND2D, PRDM8 |
| 449 | JPH1, STAT6, DENND2D, DACH1 |
| 450 | JPH1, STAT6, DENND2D, IKBKE |
| 451 | JPH1, STAT6, ETV7, DCAF13 |
| 452 | JPH1, STAT6, ETV7, PRDM8 |
| 453 | JPH1, STAT6, ETV7, DACH1 |
| 454 | JPH1, STAT6, ETV7, IKBKE |
| 455 | JPH1, STAT6, DCAF13, PRDM8 |
| 456 | JPH1, STAT6, DCAF13, DACH1 |
| 457 | JPH1, STAT6, DCAF13, IKBKE |
| 458 | JPH1, STAT6, PRDM8, DACH1 |
| 459 | JPH1, STAT6, PRDM8, IKBKE |
| 460 | JPH1, STAT6, DACH1, IKBKE |
| 461 | JPH1, DENND2D, ETV7, DCAF13 |
| 462 | JPH1, DENND2D, ETV7, PRDM8 |
| 463 | JPH1, DENND2D, ETV7, DACH1 |
| 464 | JPH1, DENND2D, ETV7, IKBKE |
| 465 | JPH1, DENND2D, DCAF13, PRDM8 |
| 466 | JPH1, DENND2D, DCAF13, DACH1 |
| 467 | JPH1, DENND2D, DCAF13, IKBKE |
| 468 | JPH1, DENND2D, PRDM8, DACH1 |
| 469 | JPH1, DENND2D, PRDM8, IKBKE |
| 470 | JPH1, DENND2D, DACH1, IKBKE |
| 471 | JPH1, ETV7, DCAF13, PRDM8 |
| 472 | JPH1, ETV7, DCAF13, DACH1 |
| 473 | JPH1, ETV7, DCAF13, IKBKE |
| 474 | JPH1, ETV7, PRDM8, DACH1 |
| 475 | JPH1, ETV7, PRDM8, IKBKE |
| 476 | JPH1, ETV7, DACH1, IKBKE |
| 477 | JPH1, DCAF13, PRDM8, DACH1 |
| 478 | JPH1, DCAF13, PRDM8, IKBKE |
| 479 | JPH1, DCAF13, DACH1, IKBKE |
| 480 | JPH1, PRDM8, DACH1, IKBKE |
| 481 | TMCO4, STAT6, DENND2D, ETV7 |
| 482 | TMCO4, STAT6, DENND2D, DCAF13 |
| 483 | TMCO4, STAT6, DENND2D, PRDM8 |
| 484 | TMCO4, STAT6, DENND2D, DACH1 |
| 485 | TMCO4, STAT6, DENND2D, IKBKE |
| 486 | TMCO4, STAT6, ETV7, DCAF13 |
| 487 | TMCO4, STAT6, ETV7, PRDM8 |
| 488 | TMCO4, STAT6, ETV7, DACH1 |
| 489 | TMCO4, STAT6, ETV7, IKBKE |
| 490 | TMCO4, STAT6, DCAF13, PRDM8 |
| 491 | TMCO4, STAT6, DCAF13, DACH1 |
| 492 | TMCO4, STAT6, DCAF13, IKBKE |
| 493 | TMCO4, STAT6, PRDM8, DACH1 |
| 494 | TMCO4, STAT6, PRDM8, IKBKE |
| 495 | TMCO4, STAT6, DACH1, IKBKE |
| 496 | TMCO4, DENND2D, ETV7, DCAF13 |
| 497 | TMCO4, DENND2D, ETV7, PRDM8 |
| 498 | TMCO4, DENND2D, ETV7, DACH1 |
| 499 | TMCO4, DENND2D, ETV7, IKBKE |
| 500 | TMCO4, DENND2D, DCAF13, PRDM8 |
| 501 | TMCO4, DENND2D, DCAF13, DACH1 |
| 502 | TMCO4, DENND2D, DCAF13, IKBKE |
| 503 | TMCO4, DENND2D, PRDM8, DACH1 |
| 504 | TMCO4, DENND2D, PRDM8, IKBKE |
| 505 | TMCO4, DENND2D, DACH1, IKBKE |
| 506 | TMCO4, ETV7, DCAF13, PRDM8 |
| 507 | TMCO4, ETV7, DCAF13, DACH1 |
| 508 | TMCO4, ETV7, DCAF13, IKBKE |
| 509 | TMCO4, ETV7, PRDM8, DACH1 |
| 510 | TMCO4, ETV7, PRDM8, IKBKE |
| 511 | TMCO4, ETV7, DACH1, IKBKE |
| 512 | TMCO4, DCAF13, PRDM8, DACH1 |
| 513 | TMCO4, DCAF13, PRDM8, IKBKE |
| 514 | TMCO4, DCAF13, DACH1, IKBKE |
| 515 | TMCO4, PRDM8, DACH1, IKBKE |
| 516 | STAT6, DENND2D, ETV7, DCAF13 |
| 517 | STAT6, DENND2D, ETV7, PRDM8 |
| 518 | STAT6, DENND2D, ETV7, DACH1 |
| 519 | STAT6, DENND2D, ETV7, IKBKE |
| 520 | STAT6, DENND2D, DCAF13, PRDM8 |
| 521 | STAT6, DENND2D, DCAF13, DACH1 |
| 522 | STAT6, DENND2D, DCAF13, IKBKE |
| 523 | STAT6, DENND2D, PRDM8, DACH1 |
| 524 | STAT6, DENND2D, PRDM8, IKBKE |
| 525 | STAT6, DENND2D, DACH1, IKBKE |
| 526 | STAT6, ETV7, DCAF13, PRDM8 |
| 527 | STAT6, ETV7, DCAF13, DACH1 |
| 528 | STAT6, ETV7, DCAF13, IKBKE |
| 529 | STAT6, ETV7, PRDM8, DACH1 |
| 530 | STAT6, ETV7, PRDM8, IKBKE |
| 531 | STAT6, ETV7, DACH1, IKBKE |
| 532 | STAT6, DCAF13, PRDM8, DACH1 |
| 533 | STAT6, DCAF13, PRDM8, IKBKE |
| 534 | STAT6, DCAF13, DACH1, IKBKE |
| 535 | STAT6, PRDM8, DACH1, IKBKE |
| 536 | DENND2D, ETV7, DCAF13, PRDM8 |
| 537 | DENND2D, ETV7, DCAF13, DACH1 |
| 538 | DENND2D, ETV7, DCAF13, IKBKE |
| 539 | DENND2D, ETV7, PRDM8, DACH1 |
| 540 | DENND2D, ETV7, PRDM8, IKBKE |
| 541 | DENND2D, ETV7, DACH1, IKBKE |
| 542 | DENND2D, DCAF13, PRDM8, DACH1 |
| 543 | DENND2D, DCAF13, PRDM8, IKBKE |
| 544 | DENND2D, DCAF13, DACH1, IKBKE |
| 545 | DENND2D, PRDM8, DACH1, IKBKE |
| 546 | ETV7, DCAF13, PRDM8, DACH1 |
| 547 | ETV7, DCAF13, PRDM8, IKBKE |
| 548 | ETV7, DCAF13, DACH1, IKBKE |
| 549 | ETV7, PRDM8, DACH1, IKBKE |
| 550 | DCAF13, PRDM8, DACH1, IKBKE |
| 551 | HLA-C, CNRIP1, JPH1, TMCO4, STAT6 |
| 552 | HLA-C, CNRIP1, JPH1, TMCO4, DENND2D |
| 553 | HLA-C, CNRIP1, JPH1, TMCO4, ETV7 |
| 554 | HLA-C, CNRIP1, JPH1, TMCO4, DCAF13 |
| 555 | HLA-C, CNRIP1, JPH1, TMCO4, PRDM8 |
| 556 | HLA-C, CNRIP1, JPH1, TMCO4, DACH1 |
| 557 | HLA-C, CNRIP1, JPH1, TMCO4, IKBKE |
| 558 | HLA-C, CNRIP1, JPH1, STAT6, DENND2D |
| 559 | HLA-C, CNRIP1, JPH1, STAT6, ETV7 |
| 560 | HLA-C, CNRIP1, JPH1, STAT6, DCAF13 |
| 561 | HLA-C, CNRIP1, JPH1, STAT6, PRDM8 |
| 562 | HLA-C, CNRIP1, JPH1, STAT6, DACH1 |
| 563 | HLA-C, CNRIP1, JPH1, STAT6, IKBKE |
| 564 | HLA-C, CNRIP1, JPH1, DENND2D, ETV7 |
| 565 | HLA-C, CNRIP1, JPH1, DENND2D, DCAF13 |
| 566 | HLA-C, CNRIP1, JPH1, DENND2D, PRDM8 |
| 567 | HLA-C, CNRIP1, JPH1, DENND2D, DACH1 |
| 568 | HLA-C, CNRIP1, JPH1, DENND2D, IKBKE |
| 569 | HLA-C, CNRIP1, JPH1, ETV7, DCAF13 |
| 570 | HLA-C, CNRIP1, JPH1, ETV7, PRDM8 |
| 571 | HLA-C, CNRIP1, JPH1, ETV7, DACH1 |
| 572 | HLA-C, CNRIP1, JPH1, ETV7, IKBKE |
| 573 | HLA-C, CNRIP1, JPH1, DCAF13, PRDM8 |
| 574 | HLA-C, CNRIP1, JPH1, DCAF13, DACH1 |
| 575 | HLA-C, CNRIP1, JPH1, DCAF13, IKBKE |
| 576 | HLA-C, CNRIP1, JPH1, PRDM8, DACH1 |
| 577 | HLA-C, CNRIP1, JPH1, PRDM8, IKBKE |
| 578 | HLA-C, CNRIP1, JPH1, DACH1, IKBKE |
| 579 | HLA-C, CNRIP1, TMCO4, STAT6, DENND2D |
| 580 | HLA-C, CNRIP1, TMCO4, STAT6, ETV7 |
| 581 | HLA-C, CNRIP1, TMCO4, STAT6, DCAF13 |
| 582 | HLA-C, CNRIP1, TMCO4, STAT6, PRDM8 |
| 583 | HLA-C, CNRIP1, TMCO4, STAT6, DACH1 |
| 584 | HLA-C, CNRIP1, TMCO4, STAT6, IKBKE |
| 585 | HLA-C, CNRIP1, TMCO4, DENND2D, ETV7 |
| 586 | HLA-C, CNRIP1, TMCO4, DENND2D, DCAF13 |
| 587 | HLA-C, CNRIP1, TMCO4, DENND2D, PRDM8 |
| 588 | HLA-C, CNRIP1, TMCO4, DENND2D, DACH1 |
| 589 | HLA-C, CNRIP1, TMCO4, DENND2D, IKBKE |
| 590 | HLA-C, CNRIP1, TMCO4, ETV7, DCAF13 |
| 591 | HLA-C, CNRIP1, TMCO4, ETV7, PRDM8 |
| 592 | HLA-C, CNRIP1, TMCO4, ETV7, DACH1 |
| 593 | HLA-C, CNRIP1, TMCO4, ETV7, IKBKE |
| 594 | HLA-C, CNRIP1, TMCO4, DCAF13, PRDM8 |
| 595 | HLA-C, CNRIP1, TMCO4, DCAF13, DACH1 |
| 596 | HLA-C, CNRIP1, TMCO4, DCAF13, IKBKE |
| 597 | HLA-C, CNRIP1, TMCO4, PRDM8, DACH1 |
| 598 | HLA-C, CNRIP1, TMCO4, PRDM8, IKBKE |
| 599 | HLA-C, CNRIP1, TMCO4, DACH1, IKBKE |
| 600 | HLA-C, CNRIP1, STAT6, DENND2D, ETV7 |
| 601 | HLA-C, CNRIP1, STAT6, DENND2D, DCAF13 |
| 602 | HLA-C, CNRIP1, STAT6, DENND2D, PRDM8 |
| 603 | HLA-C, CNRIP1, STAT6, DENND2D, DACH1 |
| 604 | HLA-C, CNRIP1, STAT6, DENND2D, IKBKE |
| 605 | HLA-C, CNRIP1, STAT6, ETV7, DCAF13 |
| 606 | HLA-C, CNRIP1, STAT6, ETV7, PRDM8 |
| 607 | HLA-C, CNRIP1, STAT6, ETV7, DACH1 |
| 608 | HLA-C, CNRIP1, STAT6, ETV7, IKBKE |
| 609 | HLA-C, CNRIP1, STAT6, DCAF13, PRDM8 |
| 610 | HLA-C, CNRIP1, STAT6, DCAF13, DACH1 |
| 611 | HLA-C, CNRIP1, STAT6, DCAF13, IKBKE |
| 612 | HLA-C, CNRIP1, STAT6, PRDM8, DACH1 |
| 613 | HLA-C, CNRIP1, STAT6, PRDM8, IKBKE |
| 614 | HLA-C, CNRIP1, STAT6, DACH1, IKBKE |
| 615 | HLA-C, CNRIP1, DENND2D, ETV7, DCAF13 |
| 616 | HLA-C, CNRIP1, DENND2D, ETV7, PRDM8 |
| 617 | HLA-C, CNRIP1, DENND2D, ETV7, DACH1 |
| 618 | HLA-C, CNRIP1, DENND2D, ETV7, IKBKE |
| 619 | HLA-C, CNRIP1, DENND2D, DCAF13, PRDM8 |
| 620 | HLA-C, CNRIP1, DENND2D, DCAF13, DACH1 |
| 621 | HLA-C, CNRIP1, DENND2D, DCAF13, IKBKE |
| 622 | HLA-C, CNRIP1, DENND2D, PRDM8, DACH1 |
| 623 | HLA-C, CNRIP1, DENND2D, PRDM8, IKBKE |
| 624 | HLA-C, CNRIP1, DENND2D, DACH1, IKBKE |
| 625 | HLA-C, CNRIP1, ETV7, DCAF13, PRDM8 |
| 626 | HLA-C, CNRIP1, ETV7, DCAF13, DACH1 |
| 627 | HLA-C, CNRIP1, ETV7, DCAF13, IKBKE |
| 628 | HLA-C, CNRIP1, ETV7, PRDM8, DACH1 |
| 629 | HLA-C, CNRIP1, ETV7, PRDM8, IKBKE |
| 630 | HLA-C, CNRIP1, ETV7, DACH1, IKBKE |
| 631 | HLA-C, CNRIP1, DCAF13, PRDM8, DACH1 |
| 632 | HLA-C, CNRIP1, DCAF13, PRDM8, IKBKE |
| 633 | HLA-C, CNRIP1, DCAF13, DACH1, IKBKE |
| 634 | HLA-C, CNRIP1, PRDM8, DACH1, IKBKE |
| 635 | HLA-C, JPH1, TMCO4, STAT6, DENND2D |
| 636 | HLA-C, JPH1, TMCO4, STAT6, ETV7 |
| 637 | HLA-C, JPH1, TMCO4, STAT6, DCAF13 |
| 638 | HLA-C, JPH1, TMCO4, STAT6, PRDM8 |
| 639 | HLA-C, JPH1, TMCO4, STAT6, DACH1 |
| 640 | HLA-C, JPH1, TMCO4, STAT6, IKBKE |
| 641 | HLA-C, JPH1, TMCO4, DENND2D, ETV7 |
| 642 | HLA-C, JPH1, TMCO4, DENND2D, DCAF13 |
| 643 | HLA-C, JPH1, TMCO4, DENND2D, PRDM8 |
| 644 | HLA-C, JPH1, TMCO4, DENND2D, DACH1 |
| 645 | HLA-C, JPH1, TMCO4, DENND2D, IKBKE |
| 646 | HLA-C, JPH1, TMCO4, ETV7, DCAF13 |
| 647 | HLA-C, JPH1, TMCO4, ETV7, PRDM8 |
| 648 | HLA-C, JPH1, TMCO4, ETV7, DACH1 |
| 649 | HLA-C, JPH1, TMCO4, ETV7, IKBKE |
| 650 | HLA-C, JPH1, TMCO4, DCAF13, PRDM8 |
| 651 | HLA-C, JPH1, TMCO4, DCAF13, DACH1 |
| 652 | HLA-C, JPH1, TMCO4, DCAF13, IKBKE |
| 653 | HLA-C, JPH1, TMCO4, PRDM8, DACH1 |
| 654 | HLA-C, JPH1, TMCO4, PRDM8, IKBKE |
| 655 | HLA-C, JPH1, TMCO4, DACH1, IKBKE |
| 656 | HLA-C, JPH1, STAT6, DENND2D, ETV7 |
| 657 | HLA-C, JPH1, STAT6, DENND2D, DCAF13 |
| 658 | HLA-C, JPH1, STAT6, DENND2D, PRDM8 |
| 659 | HLA-C, JPH1, STAT6, DENND2D, DACH1 |
| 660 | HLA-C, JPH1, STAT6, DENND2D, IKBKE |
| 661 | HLA-C, JPH1, STAT6, ETV7, DCAF13 |
| 662 | HLA-C, JPH1, STAT6, ETV7, PRDM8 |
| 663 | HLA-C, JPH1, STAT6, ETV7, DACH1 |
| 664 | HLA-C, JPH1, STAT6, ETV7, IKBKE |
| 665 | HLA-C, JPH1, STAT6, DCAF13, PRDM8 |
| 666 | HLA-C, JPH1, STAT6, DCAF13, DACH1 |
| 667 | HLA-C, JPH1, STAT6, DCAF13, IKBKE |
| 668 | HLA-C, JPH1, STAT6, PRDM8, DACH1 |
| 669 | HLA-C, JPH1, STAT6, PRDM8, IKBKE |
| 670 | HLA-C, JPH1, STAT6, DACH1, IKBKE |
| 671 | HLA-C, JPH1, DENND2D, ETV7, DCAF13 |
| 672 | HLA-C, JPH1, DENND2D, ETV7, PRDM8 |
| 673 | HLA-C, JPH1, DENND2D, ETV7, DACH1 |
| 674 | HLA-C, JPH1, DENND2D, ETV7, IKBKE |
| 675 | HLA-C, JPH1, DENND2D, DCAF13, PRDM8 |
| 676 | HLA-C, JPH1, DENND2D, DCAF13, DACH1 |
| 677 | HLA-C, JPH1, DENND2D, DCAF13, IKBKE |
| 678 | HLA-C, JPH1, DENND2D, PRDM8, DACH1 |
| 679 | HLA-C, JPH1, DENND2D, PRDM8, IKBKE |
| 680 | HLA-C, JPH1, DENND2D, DACH1, IKBKE |
| 681 | HLA-C, JPH1, ETV7, DCAF13, PRDM8 |
| 682 | HLA-C, JPH1, ETV7, DCAF13, DACH1 |
| 683 | HLA-C, JPH1, ETV7, DCAF13, IKBKE |
| 684 | HLA-C, JPH1, ETV7, PRDM8, DACH1 |
| 685 | HLA-C, JPH1, ETV7, PRDM8, IKBKE |
| 686 | HLA-C, JPH1, ETV7, DACH1, IKBKE |
| 687 | HLA-C, JPH1, DCAF13, PRDM8, DACH1 |
| 688 | HLA-C, JPH1, DCAF13, PRDM8, IKBKE |
| 689 | HLA-C, JPH1, DCAF13, DACH1, IKBKE |
| 690 | HLA-C, JPH1, PRDM8, DACH1, IKBKE |
| 691 | HLA-C, TMCO4, STAT6, DENND2D, ETV7 |
| 692 | HLA-C, TMCO4, STAT6, DENND2D, DCAF13 |
| 693 | HLA-C, TMCO4, STAT6, DENND2D, PRDM8 |
| 694 | HLA-C, TMCO4, STAT6, DENND2D, DACH1 |
| 695 | HLA-C, TMCO4, STAT6, DENND2D, IKBKE |
| 696 | HLA-C, TMCO4, STAT6, ETV7, DCAF13 |
| 697 | HLA-C, TMCO4, STAT6, ETV7, PRDM8 |
| 698 | HLA-C, TMCO4, STAT6, ETV7, DACH1 |
| 699 | HLA-C, TMCO4, STAT6, ETV7, IKBKE |
| 700 | HLA-C, TMCO4, STAT6, DCAF13, PRDM8 |
| 701 | HLA-C, TMCO4, STAT6, DCAF13, DACH1 |
| 702 | HLA-C, TMCO4, STAT6, DCAF13, IKBKE |
| 703 | HLA-C, TMCO4, STAT6, PRDM8, DACH1 |
| 704 | HLA-C, TMCO4, STAT6, PRDM8, IKBKE |
| 705 | HLA-C, TMCO4, STAT6, DACH1, IKBKE |
| 706 | HLA-C, TMCO4, DENND2D, ETV7, DCAF13 |
| 707 | HLA-C, TMCO4, DENND2D, ETV7, PRDM8 |
| 708 | HLA-C, TMCO4, DENND2D, ETV7, DACH1 |
| 709 | HLA-C, TMCO4, DENND2D, ETV7, IKBKE |
| 710 | HLA-C, TMCO4, DENND2D, DCAF13, PRDM8 |
| 711 | HLA-C, TMCO4, DENND2D, DCAF13, DACH1 |
| 712 | HLA-C, TMCO4, DENND2D, DCAF13, IKBKE |
| 713 | HLA-C, TMCO4, DENND2D, PRDM8, DACH1 |
| 714 | HLA-C, TMCO4, DENND2D, PRDM8, IKBKE |
| 715 | HLA-C, TMCO4, DENND2D, DACH1, IKBKE |
| 716 | HLA-C, TMCO4, ETV7, DCAF13, PRDM8 |
| 717 | HLA-C, TMCO4, ETV7, DCAF13, DACH1 |
| 718 | HLA-C, TMCO4, ETV7, DCAF13, IKBKE |
| 719 | HLA-C, TMCO4, ETV7, PRDM8, DACH1 |
| 720 | HLA-C, TMCO4, ETV7, PRDM8, IKBKE |
| 721 | HLA-C, TMCO4, ETV7, DACH1, IKBKE |
| 722 | HLA-C, TMCO4, DCAF13, PRDM8, DACH1 |
| 723 | HLA-C, TMCO4, DCAF13, PRDM8, IKBKE |
| 724 | HLA-C, TMCO4, DCAF13, DACH1, IKBKE |
| 725 | HLA-C, TMCO4, PRDM8, DACH1, IKBKE |
| 726 | HLA-C, STAT6, DENND2D, ETV7, DCAF13 |
| 727 | HLA-C, STAT6, DENND2D, ETV7, PRDM8 |
| 728 | HLA-C, STAT6, DENND2D, ETV7, DACH1 |
| 729 | HLA-C, STAT6, DENND2D, ETV7, IKBKE |
| 730 | HLA-C, STAT6, DENND2D, DCAF13, PRDM8 |
| 731 | HLA-C, STAT6, DENND2D, DCAF13, DACH1 |
| 732 | HLA-C, STAT6, DENND2D, DCAF13, IKBKE |
| 733 | HLA-C, STAT6, DENND2D, PRDM8, DACH1 |
| 734 | HLA-C, STAT6, DENND2D, PRDM8, IKBKE |
| 735 | HLA-C, STAT6, DENND2D, DACH1, IKBKE |
| 736 | HLA-C, STAT6, ETV7, DCAF13, PRDM8 |
| 737 | HLA-C, STAT6, ETV7, DCAF13, DACH1 |
| 738 | HLA-C, STAT6, ETV7, DCAF13, IKBKE |
| 739 | HLA-C, STAT6, ETV7, PRDM8, DACH1 |
| 740 | HLA-C, STAT6, ETV7, PRDM8, IKBKE |
| 741 | HLA-C, STAT6, ETV7, DACH1, IKBKE |
| 742 | HLA-C, STAT6, DCAF13, PRDM8, DACH1 |
| 743 | HLA-C, STAT6, DCAF13, PRDM8, IKBKE |
| 744 | HLA-C, STAT6, DCAF13, DACH1, IKBKE |
| 745 | HLA-C, STAT6, PRDM8, DACH1, IKBKE |
| 746 | HLA-C, DENND2D, ETV7, DCAF13, PRDM8 |
| 747 | HLA-C, DENND2D, ETV7, DCAF13, DACH1 |
| 748 | HLA-C, DENND2D, ETV7, DCAF13, IKBKE |
| 749 | HLA-C, DENND2D, ETV7, PRDM8, DACH1 |
| 750 | HLA-C, DENND2D, ETV7, PRDM8, IKBKE |
| 751 | HLA-C, DENND2D, ETV7, DACH1, IKBKE |
| 752 | HLA-C, DENND2D, DCAF13, PRDM8, DACH1 |
| 753 | HLA-C, DENND2D, DCAF13, PRDM8, IKBKE |
| 754 | HLA-C, DENND2D, DCAF13, DACH1, IKBKE |
| 755 | HLA-C, DENND2D, PRDM8, DACH1, IKBKE |
| 756 | HLA-C, ETV7, DCAF13, PRDM8, DACH1 |
| 757 | HLA-C, ETV7, DCAF13, PRDM8, IKBKE |
| 758 | HLA-C, ETV7, DCAF13, DACH1, IKBKE |
| 759 | HLA-C, ETV7, PRDM8, DACH1, IKBKE |
| 760 | HLA-C, DCAF13, PRDM8, DACH1, IKBKE |
| 761 | CNRIP1, JPH1, TMCO4, STAT6, DENND2D |
| 762 | CNRIP1, JPH1, TMCO4, STAT6, ETV7 |
| 763 | CNRIP1, JPH1, TMCO4, STAT6, DCAF13 |
| 764 | CNRIP1, JPH1, TMCO4, STAT6, PRDM8 |
| 765 | CNRIP1, JPH1, TMCO4, STAT6, DACH1 |
| 766 | CNRIP1, JPH1, TMCO4, STAT6, IKBKE |
| 767 | CNRIP1, JPH1, TMCO4, DENND2D, ETV7 |
| 768 | CNRIP1, JPH1, TMCO4, DENND2D, DCAF13 |
| 769 | CNRIP1, JPH1, TMCO4, DENND2D, PRDM8 |
| 770 | CNRIP1, JPH1, TMCO4, DENND2D, DACH1 |
| 771 | CNRIP1, JPH1, TMCO4, DENND2D, IKBKE |
| 772 | CNRIP1, JPH1, TMCO4, ETV7, DCAF13 |
| 773 | CNRIP1, JPH1, TMCO4, ETV7, PRDM8 |
| 774 | CNRIP1, JPH1, TMCO4, ETV7, DACH1 |
| 775 | CNRIP1, JPH1, TMCO4, ETV7, IKBKE |
| 776 | CNRIP1, JPH1, TMCO4, DCAF13, PRDM8 |
| 777 | CNRIP1, JPH1, TMCO4, DCAF13, DACH1 |
| 778 | CNRIP1, JPH1, TMCO4, DCAF13, IKBKE |
| 779 | CNRIP1, JPH1, TMCO4, PRDM8, DACH1 |
| 780 | CNRIP1, JPH1, TMCO4, PRDM8, IKBKE |
| 781 | CNRIP1, JPH1, TMCO4, DACH1, IKBKE |
| 782 | CNRIP1, JPH1, STAT6, DENND2D, ETV7 |
| 783 | CNRIP1, JPH1, STAT6, DENND2D, DCAF13 |
| 784 | CNRIP1, JPH1, STAT6, DENND2D, PRDM8 |
| 785 | CNRIP1, JPH1, STAT6, DENND2D, DACH1 |
| 786 | CNRIP1, JPH1, STAT6, DENND2D, IKBKE |
| 787 | CNRIP1, JPH1, STAT6, ETV7, DCAF13 |
| 788 | CNRIP1, JPH1, STAT6, ETV7, PRDM8 |
| 789 | CNRIP1, JPH1, STAT6, ETV7, DACH1 |
| 790 | CNRIP1, JPH1, STAT6, ETV7, IKBKE |
| 791 | CNRIP1, JPH1, STAT6, DCAF13, PRDM8 |
| 792 | CNRIP1, JPH1, STAT6, DCAF13, DACH1 |
| 793 | CNRIP1, JPH1, STAT6, DCAF13, IKBKE |
| 794 | CNRIP1, JPH1, STAT6, PRDM8, DACH1 |
| 795 | CNRIP1, JPH1, STAT6, PRDM8, IKBKE |
| 796 | CNRIP1, JPH1, STAT6, DACH1, IKBKE |
| 797 | CNRIP1, JPH1, DENND2D, ETV7, DCAF13 |
| 798 | CNRIP1, JPH1, DENND2D, ETV7, PRDM8 |
| 799 | CNRIP1, JPH1, DENND2D, ETV7, DACH1 |
| 800 | CNRIP1, JPH1, DENND2D, ETV7, IKBKE |
| 801 | CNRIP1, JPH1, DENND2D, DCAF13, PRDM8 |
| 802 | CNRIP1, JPH1, DENND2D, DCAF13, DACH1 |
| 803 | CNRIP1, JPH1, DENND2D, DCAF13, IKBKE |
| 804 | CNRIP1, JPH1, DENND2D, PRDM8, DACH1 |
| 805 | CNRIP1, JPH1, DENND2D, PRDM8, IKBKE |
| 806 | CNRIP1, JPH1, DENND2D, DACH1, IKBKE |
| 807 | CNRIP1, JPH1, ETV7, DCAF13, PRDM8 |
| 808 | CNRIP1, JPH1, ETV7, DCAF13, DACH1 |
| 809 | CNRIP1, JPH1, ETV7, DCAF13, IKBKE |
| 810 | CNRIP1, JPH1, ETV7, PRDM8, DACH1 |
| 811 | CNRIP1, JPH1, ETV7, PRDM8, IKBKE |
| 812 | CNRIP1, JPH1, ETV7, DACH1, IKBKE |
| 813 | CNRIP1, JPH1, DCAF13, PRDM8, DACH1 |
| 814 | CNRIP1, JPH1, DCAF13, PRDM8, IKBKE |
| 815 | CNRIP1, JPH1, DCAF13, DACH1, IKBKE |
| 816 | CNRIP1, JPH1, PRDM8, DACH1, IKBKE |
| 817 | CNRIP1, TMCO4, STAT6, DENND2D, ETV7 |
| 818 | CNRIP1, TMCO4, STAT6, DENND2D, DCAF13 |
| 819 | CNRIP1, TMCO4, STAT6, DENND2D, PRDM8 |
| 820 | CNRIP1, TMCO4, STAT6, DENND2D, DACH1 |
| 821 | CNRIP1, TMCO4, STAT6, DENND2D, IKBKE |
| 822 | CNRIP1, TMCO4, STAT6, ETV7, DCAF13 |
| 823 | CNRIP1, TMCO4, STAT6, ETV7, PRDM8 |
| 824 | CNRIP1, TMCO4, STAT6, ETV7, DACH1 |
| 825 | CNRIP1, TMCO4, STAT6, ETV7, IKBKE |
| 826 | CNRIP1, TMCO4, STAT6, DCAF13, PRDM8 |
| 827 | CNRIP1, TMCO4, STAT6, DCAF13, DACH1 |
| 828 | CNRIP1, TMCO4, STAT6, DCAF13, IKBKE |
| 829 | CNRIP1, TMCO4, STAT6, PRDM8, DACH1 |
| 830 | CNRIP1, TMCO4, STAT6, PRDM8, IKBKE |
| 831 | CNRIP1, TMCO4, STAT6, DACH1, IKBKE |
| 832 | CNRIP1, TMCO4, DENND2D, ETV7, DCAF13 |
| 833 | CNRIP1, TMCO4, DENND2D, ETV7, PRDM8 |
| 834 | CNRIP1, TMCO4, DENND2D, ETV7, DACH1 |
| 835 | CNRIP1, TMCO4, DENND2D, ETV7, IKBKE |
| 836 | CNRIP1, TMCO4, DENND2D, DCAF13, PRDM8 |
| 837 | CNRIP1, TMCO4, DENND2D, DCAF13, DACH1 |
| 838 | CNRIP1, TMCO4, DENND2D, DCAF13, IKBKE |
| 839 | CNRIP1, TMCO4, DENND2D, PRDM8, DACH1 |
| 840 | CNRIP1, TMCO4, DENND2D, PRDM8, IKBKE |
| 841 | CNRIP1, TMCO4, DENND2D, DACH1, IKBKE |
| 842 | CNRIP1, TMCO4, ETV7, DCAF13, PRDM8 |
| 843 | CNRIP1, TMCO4, ETV7, DCAF13, DACH1 |
| 844 | CNRIP1, TMCO4, ETV7, DCAF13, IKBKE |
| 845 | CNRIP1, TMCO4, ETV7, PRDM8, DACH1 |
| 846 | CNRIP1, TMCO4, ETV7, PRDM8, IKBKE |
| 847 | CNRIP1, TMCO4, ETV7, DACH1, IKBKE |
| 848 | CNRIP1, TMCO4, DCAF13, PRDM8, DACH1 |
| 849 | CNRIP1, TMCO4, DCAF13, PRDM8, IKBKE |
| 850 | CNRIP1, TMCO4, DCAF13, DACH1, IKBKE |
| 851 | CNRIP1, TMCO4, PRDM8, DACH1, IKBKE |
| 852 | CNRIP1, STAT6, DENND2D, ETV7, DCAF13 |
| 853 | CNRIP1, STAT6, DENND2D, ETV7, PRDM8 |
| 854 | CNRIP1, STAT6, DENND2D, ETV7, DACH1 |
| 855 | CNRIP1, STAT6, DENND2D, ETV7, IKBKE |
| 856 | CNRIP1, STAT6, DENND2D, DCAF13, PRDM8 |
| 857 | CNRIP1, STAT6, DENND2D, DCAF13, DACH1 |
| 858 | CNRIP1, STAT6, DENND2D, DCAF13, IKBKE |
| 859 | CNRIP1, STAT6, DENND2D, PRDM8, DACH1 |
| 860 | CNRIP1, STAT6, DENND2D, PRDM8, IKBKE |
| 861 | CNRIP1, STAT6, DENND2D, DACH1, IKBKE |
| 862 | CNRIP1, STAT6, ETV7, DCAF13, PRDM8 |
| 863 | CNRIP1, STAT6, ETV7, DCAF13, DACH1 |
| 864 | CNRIP1, STAT6, ETV7, DCAF13, IKBKE |
| 865 | CNRIP1, STAT6, ETV7, PRDM8, DACH1 |
| 866 | CNRIP1, STAT6, ETV7, PRDM8, IKBKE |
| 867 | CNRIP1, STAT6, ETV7, DACH1, IKBKE |
| 868 | CNRIP1, STAT6, DCAF13, PRDM8, DACH1 |
| 869 | CNRIP1, STAT6, DCAF13, PRDM8, IKBKE |
| 870 | CNRIP1, STAT6, DCAF13, DACH1, IKBKE |
| 871 | CNRIP1, STAT6, PRDM8, DACH1, IKBKE |
| 872 | CNRIP1, DENND2D, ETV7, DCAF13, PRDM8 |
| 873 | CNRIP1, DENND2D, ETV7, DCAF13, DACH1 |
| 874 | CNRIP1, DENND2D, ETV7, DCAF13, IKBKE |
| 875 | CNRIP1, DENND2D, ETV7, PRDM8, DACH1 |
| 876 | CNRIP1, DENND2D, ETV7, PRDM8, IKBKE |
| 877 | CNRIP1, DENND2D, ETV7, DACH1, IKBKE |
| 878 | CNRIP1, DENND2D, DCAF13, PRDM8, DACH1 |
| 879 | CNRIP1, DENND2D, DCAF13, PRDM8, IKBKE |
| 880 | CNRIP1, DENND2D, DCAF13, DACH1, IKBKE |
| 881 | CNRIP1, DENND2D, PRDM8, DACH1, IKBKE |
| 882 | CNRIP1, ETV7, DCAF13, PRDM8, DACH1 |
| 883 | CNRIP1, ETV7, DCAF13, PRDM8, IKBKE |
| 884 | CNRIP1, ETV7, DCAF13, DACH1, IKBKE |
| 885 | CNRIP1, ETV7, PRDM8, DACH1, IKBKE |
| 886 | CNRIP1, DCAF13, PRDM8, DACH1, IKBKE |
| 887 | JPH1, TMCO4, STAT6, DENND2D, ETV7 |
| 888 | JPH1, TMCO4, STAT6, DENND2D, DCAF13 |
| 889 | JPH1, TMCO4, STAT6, DENND2D, PRDM8 |
| 890 | JPH1, TMCO4, STAT6, DENND2D, DACH1 |
| 891 | JPH1, TMCO4, STAT6, DENND2D, IKBKE |
| 892 | JPH1, TMCO4, STAT6, ETV7, DCAF13 |
| 893 | JPH1, TMCO4, STAT6, ETV7, PRDM8 |
| 894 | JPH1, TMCO4, STAT6, ETV7, DACH1 |
| 895 | JPH1, TMCO4, STAT6, ETV7, IKBKE |
| 896 | JPH1, TMCO4, STAT6, DCAF13, PRDM8 |
| 897 | JPH1, TMCO4, STAT6, DCAF13, DACH1 |
| 898 | JPH1, TMCO4, STAT6, DCAF13, IKBKE |
| 899 | JPH1, TMCO4, STAT6, PRDM8, DACH1 |
| 900 | JPH1, TMCO4, STAT6, PRDM8, IKBKE |
| 901 | JPH1, TMCO4, STAT6, DACH1, IKBKE |
| 902 | JPH1, TMCO4, DENND2D, ETV7, DCAF13 |
| 903 | JPH1, TMC04, DENND2D, ETV7, PRDM8 |
| 904 | JPH1, TMCO4, DENND2D, ETV7, DACH1 |
| 905 | JPH1, TMCO4, DENND2D, ETV7, IKBKE |
| 906 | JPH1, TMCO4, DENND2D, DCAF13, PRDM8 |
| 907 | JPH1, TMCO4, DENND2D, DCAF13, DACH1 |
| 908 | JPH1, TMCO4, DENND2D, DCAF13, IKBKE |
| 909 | JPH1, TMCO4, DENND2D, PRDM8, DACH1 |
| 910 | JPH1, TMCO4, DENND2D, PRDM8, IKBKE |
| 911 | JPH1, TMCO4, DENND2D, DACH1, IKBKE |
| 912 | JPH1, TMCO4, ETV7, DCAF13, PRDM8 |
| 913 | JPH1, TMCO4, ETV7, DCAF13, DACH1 |
| 914 | JPH1, TMCO4, ETV7, DCAF13, IKBKE |
| 915 | JPH1, TMCO4, ETV7, PRDM8, DACH1 |
| 916 | JPH1, TMCO4, ETV7, PRDM8, IKBKE |
| 917 | JPH1, TMCO4, ETV7, DACH1, IKBKE |
| 918 | JPH1, TMCO4, DCAF13, PRDM8, DACH1 |
| 919 | JPH1, TMCO4, DCAF13, PRDM8, IKBKE |
| 920 | JPH1, TMCO4, DCAF13, DACH1, IKBKE |
| 921 | JPH1, TMCO4, PRDM8, DACH1, IKBKE |
| 922 | JPH1, STAT6, DENND2D, ETV7, DCAF13 |
| 923 | JPH1, STAT6, DENND2D, ETV7, PRDM8 |
| 924 | JPH1, STAT6, DENND2D, ETV7, DACH1 |
| 925 | JPH1, STAT6, DENND2D, ETV7, IKBKE |
| 926 | JPH1, STAT6, DENND2D, DCAF13, PRDM8 |
| 927 | JPH1, STAT6, DENND2D, DCAF13, DACH1 |
| 928 | JPH1, STAT6, DENND2D, DCAF13, IKBKE |
| 929 | JPH1, STAT6, DENND2D, PRDM8, DACH1 |
| 930 | JPH1, STAT6, DENND2D, PRDM8, IKBKE |
| 931 | JPH1, STAT6, DENND2D, DACH1, IKBKE |
| 932 | JPH1, STAT6, ETV7, DCAF13, PRDM8 |
| 933 | JPH1, STAT6, ETV7, DCAF13, DACH1 |
| 934 | JPH1, STAT6, ETV7, DCAF13, IKBKE |
| 935 | JPH1, STAT6, ETV7, PRDM8, DACH1 |
| 936 | JPH1, STAT6, ETV7, PRDM8, IKBKE |
| 937 | JPH1, STAT6, ETV7, DACH1, IKBKE |
| 938 | JPH1, STAT6, DCAF13, PRDM8, DACH1 |
| 939 | JPH1, STAT6, DCAF13, PRDM8, IKBKE |
| 940 | JPH1, STAT6, DCAF13, DACH1, IKBKE |
| 941 | JPH1, STAT6, PRDM8, DACH1, IKBKE |
| 942 | JPH1, DENND2D, ETV7, DCAF13, PRDM8 |
| 943 | JPH1, DENND2D, ETV7, DCAF13, DACH1 |
| 944 | JPH1, DENND2D, ETV7, DCAF13, IKBKE |
| 945 | JPH1, DENND2D, ETV7, PRDM8, DACH1 |
| 946 | JPH1, DENND2D, ETV7, PRDM8, IKBKE |
| 947 | JPH1, DENND2D, ETV7, DACH1, IKBKE |
| 948 | JPH1, DENND2D, DCAF13, PRDM8, DACH1 |
| 949 | JPH1, DENND2D, DCAF13, PRDM8, IKBKE |
| 950 | JPH1, DENND2D, DCAF13, DACH1, IKBKE |
| 951 | JPH1, DENND2D, PRDM8, DACH1, IKBKE |
| 952 | JPH1, ETV7, DCAF13, PRDM8, DACH1 |
| 953 | JPH1, ETV7, DCAF13, PRDM8, IKBKE |
| 954 | JPH1, ETV7, DCAF13, DACH1, IKBKE |
| 955 | JPH1, ETV7, PRDM8, DACH1, IKBKE |
| 956 | JPH1, DCAF13, PRDM8, DACH1, IKBKE |
| 957 | TMCO4, STAT6, DENND2D, ETV7, DCAF13 |
| 958 | TMCO4, STAT6, DENND2D, ETV7, PRDM8 |
| 959 | TMCO4, STAT6, DENND2D, ETV7, DACH1 |
| 960 | TMCO4, STAT6, DENND2D, ETV7, IKBKE |
| 961 | TMCO4, STAT6, DENND2D, DCAF13, PRDM8 |
| 962 | TMCO4, STAT6, DENND2D, DCAF13, DACH1 |
| 963 | TMCO4, STAT6, DENND2D, DCAF13, IKBKE |
| 964 | TMCO4, STAT6, DENND2D, PRDM8, DACH1 |
| 965 | TMCO4, STAT6, DENND2D, PRDM8, IKBKE |
| 966 | TMCO4, STAT6, DENND2D, DACH1, IKBKE |
| 967 | TMCO4, STAT6, ETV7, DCAF13, PRDM8 |
| 968 | TMCO4, STAT6, ETV7, DCAF13, DACH1 |
| 969 | TMCO4, STAT6, ETV7, DCAF13, IKBKE |
| 970 | TMCO4, STAT6, ETV7, PRDM8, DACH1 |
| 971 | TMCO4, STAT6, ETV7, PRDM8, IKBKE |
| 972 | TMCO4, STAT6, ETV7, DACH1, IKBKE |
| 973 | TMCO4, STAT6, DCAF13, PRDM8, DACH1 |
| 974 | TMCO4, STAT6, DCAF13, PRDM8, IKBKE |
| 975 | TMCO4, STAT6, DCAF13, DACH1, IKBKE |
| 976 | TMCO4, STAT6, PRDM8, DACH1, IKBKE |
| 977 | TMCO4, DENND2D, ETV7, DCAF13, PRDM8 |
| 978 | TMCO4, DENND2D, ETV7, DCAF13, DACH1 |
| 979 | TMCO4, DENND2D, ETV7, DCAF13, IKBKE |
| 980 | TMCO4, DENND2D, ETV7, PRDM8, DACH1 |
| 981 | TMCO4, DENND2D, ETV7, PRDM8, IKBKE |
| 982 | TMCO4, DENND2D, ETV7, DACH1, IKBKE |
| 983 | TMCO4, DENND2D, DCAF13, PRDM8, DACH1 |
| 984 | TMCO4, DENND2D, DCAF13, PRDM8, IKBKE |
| 985 | TMCO4, DENND2D, DCAF13, DACH1, IKBKE |
| 986 | TMCO4, DENND2D, PRDM8, DACH1, IKBKE |
| 987 | TMCO4, ETV7, DCAF13, PRDM8, DACH1 |
| 988 | TMCO4, ETV7, DCAF13, PRDM8, IKBKE |
| 989 | TMCO4, ETV7, DCAF13, DACH1, IKBKE |
| 990 | TMCO4, ETV7, PRDM8, DACH1, IKBKE |
| 991 | TMCO4, DCAF13, PRDM8, DACH1, IKBKE |
| 992 | STAT6, DENND2D, ETV7, DCAF13, PRDM8 |
| 993 | STAT6, DENND2D, ETV7, DCAF13, DACH1 |
| 994 | STAT6, DENND2D, ETV7, DCAF13, IKBKE |
| 995 | STAT6, DENND2D, ETV7, PRDM8, DACH1 |
| 996 | STAT6, DENND2D, ETV7, PRDM8, IKBKE |
| 997 | STAT6, DENND2D, ETV7, DACH1, IKBKE |
| 998 | STAT6, DENND2D, DCAF13, PRDM8, DACH1 |
| 999 | STAT6, DENND2D, DCAF13, PRDM8, IKBKE |
| 1000 | STAT6, DENND2D, DCAF13, DACH1, IKBKE |
| 1001 | STAT6, DENND2D, PRDM8, DACH1, IKBKE |
| 1002 | STAT6, ETV7, DCAF13, PRDM8, DACH1 |
| 1003 | STAT6, ETV7, DCAF13, PRDM8, IKBKE |
| 1004 | STAT6, ETV7, DCAF13, DACH1, IKBKE |
| 1005 | STAT6, ETV7, PRDM8, DACH1, IKBKE |
| 1006 | STAT6, DCAF13, PRDM8, DACH1, IKBKE |
| 1007 | DENND2D, ETV7, DCAF13, PRDM8, DACH1 |
| 1008 | DENND2D, ETV7, DCAF13, PRDM8, IKBKE |
| 1009 | DENND2D, ETV7, DCAF13, DACH1, IKBKE |
| 1010 | DENND2D, ETV7, PRDM8, DACH1, IKBKE |
| 1011 | DENND2D, DCAF13, PRDM8, DACH1, IKBKE |
| 1012 | ETV7, DCAF13, PRDM8, DACH1, IKBKE |

In some embodiments, the elevated expression of the one or more genes on the left side of Tables 2-7, or in Tables 8 and 10 (“upregulated genes”) is indicative of increased sensitivity to SVV infection. In some embodiments, the expression level of the one or more genes is at least 5%, at least 10%, at least 20%, at least 30%, at least 40%, at least 50%, at least 60%, at least 70%, at least 80%, at least 90%, at least 100%, at least 2-fold, at least 3-fold, at least 5-fold, at least 10-fold, at least 50-fold, or at least 100-fold higher than the reference gene expression level, including all ranges and subranges therebetween.

In some embodiments, the reduced expression of the one or more genes on the right side of Tables 2-7, or in Tables 9 and 11 (“downregulated genes”) is indicative of increased sensitivity to SVV infection. In some embodiments, the expression level of the one or more genes is at least 5%, at least 10%, at least 20%, at least 30%, at least 40%, at least 50%, at least 60%, at least 70%, at least 80%, at least 90%, at least 100%, at least 2-fold, at least 3-fold, at least 5-fold, at least 10-fold, at least 50-fold, or at least 100-fold lower than the reference gene expression level, including all ranges and subranges therebetween.

In some embodiments, the expression level of the one or more genes is mRNA expression level. In some embodiments, the expression level of the one or more genes is protein expression level.

### Samples

In some embodiments, the present disclosure describes obtaining a sample of the subject. In some embodiments, the subject has a cancer. In some embodiments, the sample is used for determining the expression level of the one or more genes in the cancer.

The sample may be of any biological tissue or fluid. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

In some embodiments, the sample is obtained from the subject prior to, during and/or after receiving a treatment. In some embodiments, the sample is obtained from the patient prior to the treatment. In some embodiments, the sample is obtained from the patient during the treatment, the sample is obtained from the patient after the treatment.

In some embodiments, the sample is a tissue biopsy that is embedded in paraffin wax. In some embodiments, the sample is a tissue biopsy that is fixed by Formalin. In some embodiments, the buffered formalin fixative in which biopsy specimens are processed is an aqueous solution containing 37% formaldehyde and 10-15% methyl alcohol. In some embodiments, the sample is a frozen tissue sample. The biopsy can be from any organ or tissue, for example, skin, liver, lung, heart, colon, kidney, bone marrow, teeth, lymph node, hair, spleen, brain, breast, or other organs. In a specific embodiment, the sample used in the methods described herein comprises a tumor biopsy. Any biopsy technique known by those skilled in the art can be used for isolating a sample from a subject, for instance, open biopsy, close biopsy, core biopsy, incisional biopsy, excisional biopsy, or fine needle aspiration biopsy.

In some embodiments, the sample is a bodily fluid obtained from the subject, such as blood or fractions thereof (i.e., serum, plasma), urine, saliva, sputum, or cerebrospinal fluid (CSF). In some embodiments, the sample contains cellular as well as extracellular sources of nucleic acid for use in the methods provided herein. The extracellular sources can be cell-free DNA and/or exosomes. In some embodiment, the sample can be a cell pellet or a wash. In some embodiments, the bodily fluid is blood (e.g., peripheral whole blood, peripheral blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids, including cerebrospinal fluid surrounding the brain and the spinal cord, synovial fluid surrounding bone joints, intracellular fluid, or vitreous fluids in the eyeball. In some embodiments, the sample is a blood sample.

In some embodiments, the sample comprises a plurality of cells. In some embodiments, the sample comprises stem cells, blood cells (e.g., peripheral blood mononuclear cells), lymphocytes, B cells, T cells, monocytes, granulocytes, immune cells, or tumor or cancer cells. In some embodiments, the sample comprises circulating tumor cells (CTCs).

In some embodiments, the sample comprises cell-free RNA (cfRNA).

In some embodiments, the sample comprises cells from a cell line. In some embodiments, the sample is a cell line sample.

In some embodiments, the sample is further processed before the detection of the expression levels of the genes described herein. For example, mRNA in a cell or tissue sample can be separated from other components of the sample. The sample can be concentrated and/or purified to isolate mRNA.

General methods for mRNA extraction from a sample are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™. Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes).

### Methods of Determining RNA Expression Level

Various methods of detecting or quantitating mRNA levels are known in the art. Exemplary methods include but are not limited to northern blots, ribonuclease protection assays, PCR-based methods, sequencing methods, and the like. The mRNA sequence can be used to prepare a probe that is at least partially complementary. The probe can then be used to detect the mRNA sequence in a sample, using any suitable assay, such as PCR-based methods, Northern blotting, a dipstick assay, and the like. In some embodiments, mRNA expression in a sample is quantified by northern blotting and in situ hybridization, RNAse protection assays, nCounter® Analysis, or PCR-based methods such as RT-PCR. Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label.

mRNA level may be determined by hybridization methods using corresponding probes. Hybridization is typically performed under stringent hybridization conditions. Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art. In some embodiments, mRNA from the sample is hybridized to a synthetic DNA probe. In some embodiments, the probe comprises a detection moiety (e.g., detectable label, capture sequence, barcode reporting sequence).

In some embodiments, Real-Time Reverse Transcription-PCR (RT-qPCR) can be used for both the detection and quantification of mRNA.

In some embodiments, the mRNA expression level can be measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™).

In some embodiments, the mRNA expression level is measured using a microarray and/or gene chip. In certain embodiments, the amount of one, two, three or more RNA transcripts is determined by RT-PCR.

In some embodiments, NanoString (e.g., nCounter® miRNA Expression Assays provided by NanoString® Technologies) is used for analyzing the mRNA expression level.

In some embodiments, the present disclosure can use RNA-seq by Expected Maximization (RSEM) to quantify gene expression levels from TCGA RNA-seq data.

In some embodiments, once the mRNA is obtained from a sample, it is converted to complementary DNA (cDNA) in a hybridization reaction. In some embodiments, the cDNA is a non-natural molecule. Conversion of the mRNA to cDNA can be performed with oligonucleotides or primers comprising sequence that is complementary to a portion of a specific mRNA. In some embodiments, cDNA is amplified with primers that introduce an additional DNA sequence (adapter sequence).

In some embodiments, the synthesized cDNA (for example, amplified cDNA) is immobilized on a solid surface via hybridization with a probe, e.g., via a microarray. In some embodiments, cDNA products are detected via real-time polymerase chain reaction (PCR) via the introduction of fluorescent probes that hybridize with the cDNA products. For example, in some embodiments, biomarker detection is assessed by quantitative fluorogenic RT-PCR (e.g., with TaqMan® probes).

In some embodiments, the expression level of the mRNA is determined by a fragment of the mRNA. In some embodiments, the fragment comprises a polynucleotide having at least 50, 75, 100, 150, 200, 250, 300, 350, 400, 450, 500, 550, 600, 650, 700, 800, 900, 1,000, 1,200, or 1,500 contiguous nucleotides that match or complement to the corresponding mRNA.

In some embodiments, the expression level of the mRNA is determined by normalization to the level of reference RNA transcripts, which can be all measured transcripts in the sample or a reference RNA transcript. Normalization is performed to correct for or normalize away both differences in the amount of RNA or cDNA assayed and variability in the quality of the RNA or cDNA used. Therefore, an assay may measure and incorporate the expression of certain reference genes, including well known housekeeping genes, such as, for example, GAPDH and/or β-Actin.

### Methods of Determining Protein Expression Level

Various protein detection and quantitation methods can be used to measure the expression level of proteins. Exemplary methods that can be used include but are not limited to immunoblotting (e.g., western blot), immunohistochemistry, enzyme-linked immunosorbent assay (ELISA), flow cytometry, cytometric bead array, mass spectroscopy, proteomics-based methods, and the like. Several types of ELISA are commonly used, including direct ELISA, indirect ELISA, and sandwich ELISA. In some embodiments, antibody-based methods are used.

In some embodiments, the expression level of the protein is determined by a fragment of the protein. In some embodiments, the fragment comprises a polynucleotide having at least 5, 6, 7, 8, 9, 10, 15, 20, 25, 50, 75, 100, 150, or 200 contiguous amino acids that match or complement to the corresponding protein.

In some embodiments, the expression level of the protein is determined by normalization to the level of reference protein, which can be all measured protein in the sample or a reference protein. Normalization is performed to correct for or normalize away both differences in the amount and variability of protein assayed. Therefore, an assay may measure and incorporate the expression of certain reference protein, including protein products of well-known housekeeping genes, such as, for example, GAPDH and/or β-Actin.

### Kit

In one aspect, provided herein are kits comprising reagents for determining the expression level of one or more genes described herein in a sample. In some embodiments, the sample is in a cancer sample obtained from a subject. In some embodiments, the kits comprise instructions for use. In some embodiments, the instructions provide a reference score and/or a reference level of gene expression and/or an output of a functional transformation applied to expression that a gene or subset of genes needs to be achieved in order to indicate that cancer will be sensitive to the oncolytic virus described herein. In some embodiments, the kit is for cancer diagnosis and/or characterization. In some embodiments, the kit is for selecting a subject for cancer treatment. In some embodiments, the kit is for determining whether a subject is suitable for cancer treatment.

In some embodiments, the disclosure provides the use of a kit in the manufacture of a medicament for treating cancer. In some embodiments, the kit is used for companion diagnostics associated with a medicament (e.g., a composition comprising SVV or a polynucleotide encoding the SVV viral genome).

In some embodiments, the kit comprises a solid support, and a means for detecting the RNA or protein expression of at least one gene in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In some embodiment, the kit comprises components for isolating RNA. In some embodiment, the kit comprises components for conducting RT-PCR, RT-qPCR, deep sequencing, or a microarray such as NanoString assay. In some embodiments, the kit comprises a solid support, nucleic acids contacting the support, wherein the nucleic acids are complementary to at least 10, 20, 30, 50, 70, 100, 200, or more bases of mRNA, and a means for detecting the expression of the mRNA in a biological sample.

In some embodiments, the kit comprises a microarray, wherein the microarray is comprised of oligonucleotides and/or DNA and/or RNA fragments which hybridize to one or more of the products of one or more of the genes or a subset of genes of the disclosure. In some embodiments, such kits may include primers for PCR of either the RNA product or the cDNA copy of the RNA product of the genes or subset of genes, or both. In some embodiments, such kits may include primers for PCR as well as probes for Quantitative PCR. In some embodiments, such kits may include multiple primers and multiple probes wherein some of said probes have different flourophores so as to permit multiplexing of multiple products of a gene product or multiple gene products. In some embodiments, such kits may further include materials and reagents for creating cDNA from RNA. In some embodiments, such kits may include a computer program product embedded on computer readable media for predicting whether a cancer is sensitive to SVV.

In some embodiments, the kit comprises components for isolating protein. In some embodiments, the kit comprises components for conducting flow cytometry or an ELISA. In some embodiments, the kit comprises one or more antibodies. For antibody-based kits, the kit can comprise, for example: (1) a first antibody (which may or may not be attached to a solid support) which binds to a peptide, polypeptide or protein of interest; and, optionally, (2) a second, different antibody which binds to either the peptide, polypeptide or protein, or the first antibody and is conjugated to a detectable label (e.g., a fluorescent label, radioactive isotope or enzyme). In some embodiments, the peptide, polypeptide or protein of interest is associated with or indicative of a condition (e.g., a disease). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

### Seneca Valley Virus (SVV)

In one aspect, the present disclosure provides methods of determining the sensitivity of a cancer to SVV infection and treating the cancer with SVV if the cancer is determined to be sensitive to SVV infection. In some embodiments, the SVV comprises a SVV viral particle. See, e.g., International PCT Publication Nos. WO 2021/016194 and WO 2020/210711, and U.S. Pat. No. 10,537,599. In some embodiments, SVV infection comprises administering a particle (e.g., a lipid nanoparticle) encapsulating a polynucleotide (e.g., a recombinant RNA molecule) encoding SVV. See, e.g., International PCT Publication No. WO 2019/014623 and WO 2020/142725. In some embodiments, SVV infection comprises administering a lipid nanoparticle which encapsulates an SVV viral genome. See, e.g., International PCT Publication No. WO 2020/142725.

The SVV of the disclosure maybe a derivative of SVV. As used herein, the terms “derivative” used in reference to a virus can have a viral genome or a viral protein substantially different than a template viral genome or viral protein described herein. In some embodiments, the SVV derivative is a SVV mutant, a SVV variant, a modified SVV comprising a transgene, or chimeric virus derived partly from SVV. In some embodiments, the SVV derivative is modified to be capable of recognizing different cell receptors (e.g., various cancer antigens or neoantigens). In some embodiments, the SVV derivative is modified to be capable of evading the immune system while still being able to infect, replicate in and kill the cell of interest (e.g., cancer cell). In some embodiments, the SVV derivative is a pseudotyped virus.

In some embodiments, the SVV viral genomes comprises a polynucleotide sequence at least 80%, at least 85%, at least 90%, at least 95%, at least 98%, at least 99%, or 100% identical to one of SEQ ID NO: 1-4.

In some embodiments, the RNA viral genomes described herein encode a chimeric picornavirus (e.g., encode a virus comprising one portion, such as a capsid protein or an IRES, derived from a first picornavirus and another portion derived from a first picornavirus, and another portion, a non-structural gene such as a protease or polymerase derived from a second picornavirus). In some embodiments, the first picornavirus is SVV. In some embodiments, the RNA viral genomes described herein encode a chimeric SVV.

In some embodiments, the SVV RNA viral genome comprises a microRNA (miRNA) target sequence (miR-TS) cassette, wherein the miR-TS cassette comprises one or more miRNA target sequences, and wherein expression of one or more of the corresponding miRNAs in a cell inhibits replication of the encoded oncolytic virus in the cell. Such embodiments are described, for example, in International PCT Publication No. WO 2020/142725.

In some embodiments, the SVV RNA viral genome comprises a heterologous polynucleotide encoding a payload molecule. In some embodiments, the payload molecule is selected from IL-12, GM-CSF, CXCL10, IL-36γ, CCL21, IL-18, IL-2, CCL4, CCL5, an anti-CD3-anti-FAP BiTE, an antigen binding molecule that binds DLL3, or an antigen binding molecule that binds EpCAM. Such embodiments are described, for example, in International PCT Publication No. WO 2020/142725.

### Pharmaceutical Compositions and Methods of Use

One aspect of the disclosure relates to administration of pharmaceutical compositions comprising the SVV, or the polynucleotide encoding the SVV viral genome (e.g., encapsulated in a particle of the disclosure), and methods for the treatment of cancer.

Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

In some embodiments, the pharmaceutical composition is formulated for systemic administration. In some embodiments, the systemic administration comprises intravenous administration, intra-arterial administration, intraperitoneal administration, intramuscular administration, intradermal administration, subcutaneous administration, intranasal administration, oral administration, or a combination thereof. In some embodiments, the pharmaceutical composition is formulated for intravenous administration. In some embodiments, the pharmaceutical composition is formulated for local administration. In some embodiments, the pharmaceutical composition is formulated for intratumoral administration.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement or may be an improvement in the general feeling of well-being of the subject. Thus, one of skill in the art realizes that a treatment may improve the disease condition but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival, or improvements in the quality of life.

SVV or the polynucleotide encoding the SVV viral genome may be administered to a subject in an amount that is effective to inhibit, prevent of destroy the growth of the tumor cells through replication of the virus in the tumor cells. Administration of SVV for cancer therapy include systemic, regional or local delivery of the virus at safe, developable, and tolerable doses to elicit therapeutically useful destruction of tumor cells. In some embodiments, the therapeutic index for SVV following systemic administration, is at least 10, preferably at least 100 or more preferably at least 1000. In some embodiments, SVV is administered in an amount of between 1×10⁷ and 1×10¹¹ viral genome/kg, for example, about 1×10⁷ viral genome/kg, about 1×10⁸ viral genome/kg, about 1×10⁹ viral genome/kg, about 1×10¹⁰ viral genome/kg, or about 1×10¹¹ viral genome/kg. The exact dosage to be administered may depend on a variety of factors including the age, weight, and sex of the patient, and the size and severity of the tumor being treated. The viruses may be administered one or more times, which may be dependent upon the immune response potential of the host. Single or multiple administrations of the compositions can be carried out with dose levels and pattern being selected by the treating physician. If necessary, the immune response may be diminished by employing a variety of immunosuppressants, so as to permit repetitive administration and/or enhance replication by reducing the immune response to the viruses. Anti-cancer viral therapy may be combined with other anti-cancer protocols. Delivery can be achieved in a variety of ways, employing liposomes, direct injection, catheters, topical application, inhalation, intravenous delivery, etc. Further, a DNA copy of the SVV genomic RNA, or portions thereof, can also be a method of delivery, where the DNA is subsequently transcribed by cells to produce SVV virus particles or particular SVV polypeptides. See e.g., International PCT Publication No. WO 2019/014623.

In some embodiments, the therapeutically effective amount of a composition of the disclosure is between about 1 ng/kg body weight to about 100 mg/kg body weight. In some embodiments, the range of a composition of the disclosure administered is from about 1 ng/kg body weight to about 1 μg/kg body weight, about 1 ng/kg body weight to about 100 ng/kg body weight, about 1 ng/kg body weight to about 10 ng/kg body weight, about 10 ng/kg body weight to about 1 μg/kg body weight, about 10 ng/kg body weight to about 100 ng/kg body weight, about 100 ng/kg body weight to about 1 μg/kg body weight, about 100 ng/kg body weight to about 10 μg/kg body weight, about 1 μg/kg body weight to about 10 μg/kg body weight, about 1 μg/kg body weight to about 100 μg/kg body weight, about 10 μg/kg body weight to about 100 μg/kg body weight, about 10 μg/kg body weight to about 1 mg/kg body weight, about 100 μg/kg body weight to about 10 mg/kg body weight, about 1 mg/kg body weight to about 100 mg/kg body weight, or about 10 mg/kg body weight to about 100 mg/kg body weight. Dosages within this range can be achieved by single or multiple administrations, including, e.g., multiple administrations per day or daily, weekly, bi-weekly, or monthly administrations. Compositions of the disclosure may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. In some embodiments, a composition of the disclosure is administered weekly. In some embodiments, a composition of the disclosure is administered biweekly. In some embodiments, a composition of the disclosure is administered every three weeks. However, other dosage regimens may be useful. The progress of this therapy is easily monitored by conventional techniques.

The regimen of administration may affect what constitutes an effective amount. For example, the therapeutic formulations may be administered to the patient subject either prior to or after a surgical intervention related to cancer, or shortly after the patient was diagnosed with cancer. Further, several divided dosages, as well as staggered dosages may be administered sequentially, or the dose may be continuously infused, or may be a bolus injection. Further, the dosages of the therapeutic formulations may be proportionally increased or decreased as indicated by the exigencies of the therapeutic or prophylactic situation.

Toxicity and therapeutic efficacy of viruses can be determined by standard procedures in cell cultures or experimental animals, e.g., for determining the LD50 (the dose lethal to 50% of the population of animals or cells; for viruses, the dose is in units of vp/kg) and the ED50 (the dose effective in 50% of the population of animals or cells) or the EC50 (the effective concentration in 50% of the population of animals or cells). The dose ratio between toxic and therapeutic effects is the therapeutic index and it can be expressed as the ratio between LD50 and ED50 or EC50. Viruses which exhibit high therapeutic indices are preferred. The data obtained from cell culture assays and animal studies can be used in formulating a range of dosage for use in human. The dosage of viruses lies preferably within a range of circulating concentrations that include the ED50 or EC50 with little or no toxicity. The dosage may vary within this range depending upon the dosage form employed and the route of administration utilized.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

As used herein “prevention” or “prophylaxis” can mean complete prevention of the symptoms of a disease, a delay in onset of the symptoms of a disease, or a lessening in the severity of subsequently developed disease symptoms.

The term “subject” or “patient” as used herein, is taken to mean any mammalian subject to which a composition described herein is administered according to the methods described herein. In some embodiments, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish, and reptiles.

In prophylactic applications, pharmaceutical compositions are administered to a subject susceptible to, or otherwise at risk of, a particular disorder in an amount sufficient to eliminate or reduce the risk or delay the onset of the disorder. In therapeutic applications, compositions are administered to a subject suspected of, or already suffering from such a disorder in an amount sufficient to cure, or at least partially arrest, the symptoms of the disorder and its complications.

A pharmaceutical composition may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

Dosage of the pharmaceutical composition can be varied by the attending clinician to maintain a desired concentration at a target site. Higher or lower concentrations can be selected based on the mode of delivery. Dosage should also be adjusted based on the release rate of the administered formulation.

Compositions of the disclosure may be administered as the sole treatment, as a monotherapy, or in conjunction with other drugs or therapies, as a combinatorial therapy, useful in treating the condition in question.

In some embodiments, the pharmaceutical composition of the disclosure is administered to a subject for multiple times (e.g., multiple doses). In some embodiments, the pharmaceutical composition is administered two or more times, three or more times, four or more times, etc. In some embodiments, administration of the pharmaceutical composition may be repeated once, twice, 3, 4, 5, 6, 7, 8, 9, 10, or more times. The pharmaceutical composition may be administered chronically or acutely, depending on its intended purpose.

In some embodiments, the interval between two consecutive doses of the pharmaceutical composition is less than 4, less than 3, less than 2, or less than 1 weeks. In some embodiments, the interval between two consecutive doses is less than 3 weeks. In some embodiments, the interval between two consecutive doses is less than 2 weeks. In some embodiments, the interval between two consecutive doses is less than 1 week. In some embodiments, the interval between two consecutive doses is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive doses of the pharmaceutical composition is at least 4, at least 3, at least 2, or at least 1 weeks. In some embodiments, the interval between two consecutive doses of the pharmaceutical composition of the disclosure is at least 3 weeks. In some embodiments, the interval between two consecutive doses of the pharmaceutical composition of the disclosure is at least 2 weeks. In some embodiments, the interval between two consecutive doses of the pharmaceutical composition of the disclosure is at least 1 week. In some embodiments, the interval between two consecutive doses of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the subject is administered a dose of the pharmaceutical composition of the disclosure once daily, every 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the subject is administered a dose of the pharmaceutical composition of the disclosure once every 4, 5, 6, 7, 8, 9, 10, 11, or 12 weeks. In some embodiments, the subject is administered a dose of the pharmaceutical composition of the disclosure once every 1, 2, 3, 4, 5, 6, 7, 8, 9, 10, 11, or 12 months.

In some embodiments, administration of the pharmaceutical composition of the disclosure to a subject bearing a tumor inhibits growth of the tumor. In some embodiments, administration of the pharmaceutical composition inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, administration of the pharmaceutical composition to a subject bearing a tumor leads to tumor shrinkage or elimination. In some embodiments, administration of the pharmaceutical composition leads to tumor shrinkage or elimination for at least 1 week, at least 1 month, at least 2 months, at least 3 months, at least 4 months, at least 6 months, at least 9 months, at least 12 months, at least 2 years, or longer. In some embodiments, administration of the pharmaceutical composition leads to tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

### Cancer

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is a neuroendocrine cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein. In some embodiments, the cancer is selected from small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), and glioblastoma multiforme-IDH mutant (GBM-IDH mutant).

In some embodiments, the cancer is a metastatic cancer. In some embodiments, the cancer has metastasized. In some embodiments, the cancer is a non-metastatic cancer.

In some embodiments, the cancer is selected from the group consisting of lung cancer, breast cancer, colon cancer, pancreatic cancer, bladder cancer, renal cell carcinoma, ovarian cancer, gastric cancer and liver cancer. In some embodiments, the cancer is renal cell carcinoma, lung cancer, or liver cancer. In some embodiments, the lung cancer is NSCLC (non-small cell lung cancer). In some embodiments, the liver cancer is HCC (hepatocellular carcinoma). In some embodiments, the liver cancer is metastatic. In some embodiments, the breast cancer is TNBC (triple-negative breast cancer). In some embodiments, the bladder cancer is urothelial carcinoma. In some embodiments, the cancer is selected from the group consisting of breast cancer, esophageal cancer, stomach cancer, lung cancer, kidney cancer and skin cancer, and wherein the cancer has metastasized into liver. In some embodiments, the cancer is a metastasized cancer in the liver, wherein the cancer is originated from the group consisting of breast cancer, esophageal cancer, stomach cancer, lung cancer, kidney cancer and skin cancer.

In some embodiments, the cancer is lung cancer, liver cancer, prostate cancer, bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma (MCC), or melanoma. In some embodiments, the cancer is neuroblastoma. In some embodiments, the cancer is small cell lung cancer (SCLC). In some embodiments, the cancer is rhabdomyosarcoma.

In some embodiments, the cancer is small cell lung cancer (SCLC). In some embodiments, the SCLC is ASCL1+, NeuroD1+, POU2F3+, and/or YAP1+ subtype. In some embodiments, the SCLC is NeuroD1+ subtype.

In some embodiments, the cancer is metastatic liver cancer.

In some embodiments, the cancer is Merkel cell carcinoma (MCC).

In some embodiments, the cancer is a neuroendocrine cancer. In some embodiments, the cancer is large cell neuroendocrine carcinoma (LCNEC).

In some embodiments, the cancer is a prostate cancer. In some embodiments, the cancer is castration-resistant prostate cancer. In some embodiments, the cancer is castration-resistant prostate cancer with neuroendocrine phenotype (CRPC-NE).

In some embodiments, the cancer has been previously treated with one or more therapeutic agents. In some embodiments, the cancer has relapsed after the treatment of the therapeutic agent. In some embodiments, the therapeutic agent is a chemotherapeutic agent, a kinase inhibitor, a checkpoint inhibitor, or a PARP inhibitor. In some embodiments, subjects are selected for treatment according to the methods described herein, wherein the subject has previously received treatment with a therapeutic agent (e.g., a chemotherapeutic agent, a kinase inhibitor, a checkpoint inhibitor, or a PARP inhibitor).

In some embodiments, the therapeutic agent is a chemotherapeutic agent. In some embodiments, the chemotherapeutic agent is selected from an alkylating agent, an antimetabolite, an anthracycline, a platinum-based agent, a plant alkaloid, a topoisomerase inhibitor, a vinca alkaloid, a taxane, and an epipodophyllotoxin. In some embodiments, the chemotherapeutic agent is a platinum-based chemotherapeutic agent. In some embodiments, the chemotherapeutic agent is Cisplatin.

In some embodiments, the therapeutic agent is a checkpoint kinase inhibitor. In some embodiments, the checkpoint kinase inhibitor is selected from AZD7762, SCH900776/MK-8776, IC83/LY2603618, LY2606368 (Prexasertib), GDC-0425, PF-00477736, XL844, CEP-3891, SAR-020106, CCT-244747, Arry-575, and SB218075. Additional checkpoint kinase inhibitors are described in US 2018/0344655, the content of which is incorporated by reference in its entirety. In some embodiments, the checkpoint inhibitor is Prexasertib.

In some embodiments, the therapeutic agent is a Poly (ADP-ribose) polymerase (PARP) inhibitor. In some embodiments, the PARP inhibitor is selected from olaparib, rucaparib, niraparib, talazoparib, iniparib and veliparib. Additional PARP inhibitors are described in US 2020/0407720, the content of which is incorporated by reference in its entirety. In some embodiments, the PARP inhibitor is Talazoparib.

In some embodiments, the disclosure provides methods of treating a cancer in a subject comprising administering to a subject suffering from the cancer (i) an effective amount of the virus or a polynucleotide encoding the virus, or compositions thereof, of the disclosure, and (ii) an effective amount of a second therapeutic agent.

In some embodiments, both of 1) the virus or a polynucleotide encoding the virus, or compositions thereof, and 2) the second therapeutic agent are concurrently administered. In some embodiments, these two therapeutic components are administered sequentially. In some embodiments, one or both therapeutic components are administered multiple times.

In some embodiments, the second therapeutic agent is selected from the group consisting of an immune checkpoint inhibitor, a JAK/STAT inhibitor, an mTOR inhibitor, an interferon (IFN) pathway inhibitor, and an HDAC inhibitor.

In some embodiments, the second therapeutic agent is a immune checkpoint inhibitor. In some embodiments, the immune checkpoint inhibitor is an antibody or an antigen binding fragment thereof. In some embodiments, the immune checkpoint inhibitor binds to PD-1 (e.g., the inhibitor is an anti-PD-1 antibody). Anti-PD1 antibodies are known in the art, for example, Nivolumab, Pembrolizumab, Lambrolizumab, Pidilzumab, Cemiplimab, and AMP-224 (AstraZeneca/MedImmune and GlaxoSmithKline), JTX-4014 by Jounce Therapeutics, Spartalizumab (PDR001, Novartis), Camrelizumab (SHR1210, Jiangsu HengRui Medicine Co., Ltd), Sintilimab (IBI308, Innovent and Eli Lilly), Tislelizumab (BGB-A317), Toripalimab (JS 001), Dostarlimab (TSR-042, WBP-285, GlaxoSmithKline), INCMGA00012 (MGA012, Incyte and MacroGenics), and AMP-514 (MEDI0680, AstraZeneca). In some embodiments, the immune checkpoint inhibitor binds to PD-L1 (e.g., the inhibitor is an anti-PD-L1 antibody). Anti-PDL1 antibodies are known in the art, for example, MEDI-4736, MPDL3280A, Atezolizumab (Tecentriq, Roche Genentech), Avelumab (Bavencio, Merck Serono and Pfizer), and Durvalumab (Imfinzi, AstraZeneca). In some embodiments, the immune checkpoint inhibitor binds to CTLA4 (e.g., the inhibitor is an anti-CTLA4 antibody). Anti-CTLA4 antibodies are known in the art, for example, ipilumumab, tremelimumab, or any of the antibodies disclosed in WO2014/207063. In some embodiments, the immune checkpoint inhibitor is an anti-TIGIT antibody or fragment thereof. Anti-TIGIT antibodies are known in the art, for example tiragolumab (Roche), EOS-448 (iTeos Therapeutics), Vibostolimab (Merck), Domvanalimab (Arcus, Gilead), BMS-986207 (BMS), Etigilimab (Mereo), COM902 (Compugen), ASP8374 (Astellas), SEA-TGT (Seattle Genetics) BGB-A1217 (BeiGene), IBI-939 (Innovent), and M6223 (EMD Serono).

In some embodiments, the second therapeutic agent is a JAK/STAT inhibitor. In some embodiments, the JAK/STAT inhibitor is selected from ruxolitinib, tofacitinib, oclacitinib, baricitinib, filgotinib, gandotinib, lestaurtinib, momelotinib, pacritinib, PF-04965842, upadacitinib, peficitinib, fedratinib, cucurbitacin I, decernotinib, INCB018424, AC430, BMS-0911543, GSK2586184, VX-509, R348, AZD1480, CHZ868, PF-956980, AG490, WP-1034, JAK3 inhibitor IV, atiprimod, FM-381, SAR20347, AZD4205, ARN4079, NIBR-3049, PRN371, PF-06651600, PF-06700841, NC1153, EP009, Gingerenone A, JANEX-1, cercosporamide, JAK3-IN-2, PF-956980, Tyk2-IN-30, Tyk2-IN-2, JAK3-IN1, WHI-P97, TG-101209, AZ960, NVP-BSK805, NSC 42834, FLLL32, SD 1029, WIH-P154, WHI-P154, TCS21311, JAK3-IN-1, JAK3-IN-6, JAK3-IN-7, XL019, MS-1020, AZD1418, WP1066, CEP33779, ZM 449829, SHR0302, JAK1-IN-31, WYE-151650, EXEL-8232, solcitinib, itacitinib, cerdulatinib, PF-06263276, delgotinib, AS2553627, JAK-IN-35, ASN-002, AT9283, diosgenin, JAK-IN-1, LFM-A13, NS-018, RGB-286638, SB1317, curcumol, Go6976, JAK2 inhibitor G5-7, and myricetin. Additional JAK/STAT inhibitors are described in US 2020/0281857, the content of which is incorporated by reference in its entirety.

In some embodiments, the second therapeutic agent an mTOR inhibitor. In some embodiments, the mTOR inhibitor is selected from tacrolimus, temsirolimus, everolimus, rapamycin, ridaforolimus, AZD8055, Ku-0063794, PP242, PP30, Torinl, WYE-354, PI-103, BEZ235, PKI-179, LY3023414, omipalisib, sapanisertib, OSI-027, RapaLink-1 and voxtalisib. Additional mTOR inhibitors are described in US 2018/0085362, the content of which is incorporated by reference in its entirety.

In some embodiments, the second therapeutic agent is an interferon (IFN) pathway inhibitor. In some embodiments, the IFN pathway inhibitor is an antagonist of IFN or IFN receptor. In some embodiments, the IFN pathway inhibitor is an anti-IFN antibody or the antigen binding fragment thereof. In some embodiments, the IFN pathway inhibitor is an anti-IFN receptor antibody or the antigen binding fragment thereof.

In some embodiments, the second therapeutic agent is an HDAC inhibitor. In some embodiments, the HDAC inhibitor is selected from Vorinostat/suberoyl anilide hydroxamic acid, JNJ-26481585 (N-hydroxy-2-(4-((((1-methyl-1H-indol-3-yl)methyl)amino)methyl)piperidin-1-yl)pyrimidine-5-carboxamide), R306465/JM-16241199 (N-hydroxy-5-(4-(naphthalen-2-ylsulfonyl)piperazin-1-yl)pyrimidine-2-carboxamide), CHR-3996 (2-(6-{[(6-Fluoroquinolin-2-yl)methyl]amino}-3-azabicyclo[3.1.0]hex-3-yl)-N-hydroxypyrimidine-5-carboxamide), Belinostat/PXD101, Panobinostat/LBH-589, trichostatin A/TSA (7-[4-(dimethylamino)phenyl]-N-hydroxy-4,6-dimethyl-7-oxohepta-2,4-dienamide), ITF2357, CBHA, Givinostat/ITF2357, PCI-24781, depsipeptides, romidepsin, butyrate, phenylbutyrate, valproic acid, AN-9, CI-994, Entinostat/MS-275/SNDX-275, mocetinostat/MGCD0103 (N-(2-aminophenyl)-4-((4-pyridin-3-ylpyrimidin-2-ylamino)methyl)benzamide), m-carboxycinnamic acid, bishydroxamic acid, suberic bishydroxamic acid, oxamflatin, ABHA, SB-55629, pyroxamide, propenamides, aroyl pyrrolyl hydroxamides, or LAQ824 (((E)-N-hydroxy-3-[4-[[2-hydroxyethyl-[2-(1H-indol-3 yl) ethyl]amino] methyl]phenyl] prop-2-enamide), chidamide, and 4SC-202. Additional HDAC inhibitors are described in US 2019/0290646, the content of which is incorporated by reference in its entirety.

### Further Number Embodiments

Further numbered embodiments of the invention are provided as follows:

Embodiment 1. A method of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer, and wherein the one or more genes comprise at least one of the genes listed in one of Tables 1-14 or a combination thereof.

Embodiment 2. A method of treating a cancer in a subject in need thereof, comprising:
(a) determining the expression level of one or more genes in the cancer;
(b) classifying the cancer as sensitive to Seneca Valley Virus (SVV) infection based on the expression level of the one or more genes as determined in (a); and
(c) administering a therapeutically effective amount of a Seneca Valley Virus (SVV) or a polynucleotide encoding the SVV viral genome to the subject if the cancer is classified as sensitive to SVV infection in step (b),
wherein the one or more genes comprise at least one of the genes listed in one of Tables 1-14 or a combination thereof.

Embodiment 3. A method of evaluating the sensitivity of a cancer to Seneca Valley Virus (SVV) infection, comprising determining the expression level of one or more genes in the cancer, wherein the one or more genes comprise at least one of the genes listed in one of Tables 1-14 or a combination thereof, and classifying the cancer as sensitive or resistant to SVV infection based on the expression level of the one or more genes.

Embodiment 4. The method of Embodiment 3, comprising administering a SVV or a polynucleotide encoding the SVV viral genome to the cancer.

Embodiment 5. A method of selecting a subject suffering from a cancer for treatment with a Seneca Valley Virus (SVV) or a polynucleotide encoding the SVV viral genome, comprising:
(a) determining the expression level of one or more genes in the cancer, wherein the one or more genes comprise at least one of the genes listed in one of Tables 1-14 or a combination thereof;
(b) classifying the cancer as sensitive to SVV infection based on the expression level of the one or more genes as determined in (a); and
(c) selecting the subject for treatment with the SVV or the polynucleotide encoding the SVV viral genome if the cancer is classified as sensitive to SVV infection in (b).

Embodiment 6. The method of Embodiment 5, comprising: (d) administering the SVV or the polynucleotide encoding the SVV viral genome to the selected subject.

Embodiment 7. A method of determining the expression level of one or more genes in a cancer, wherein the one or more genes comprise at least one of the genes listed in one of Tables 1-14 or a combination thereof.

Embodiment 8. The method of any one of Embodiments 1-7, wherein the one or more genes comprise at least one gene selected from one of Tables 2-7.

Embodiment 9. The method of any one of Embodiments 1-7, wherein the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 genes selected from one of Tables 1-14.

Embodiment 10. The method of any one of Embodiments 1-9, wherein the one or more genes have a frequency of at least 5% in Table 2 or 3.

Embodiment 11. The method of any one of Embodiments 1-9, wherein the one or more genes have a frequency of at least 10%, 15%, 20%, 25%, 30%, 35%, 40%, 45%, 50%, 55%, 60%, 65%, 70%, 75%, or 80% in Table 2 or 3.

Embodiment 12. The method of any one of Embodiments 1-11, wherein the one or more genes comprise all genes having a frequency of at least 40% in Table 2 or 3.

Embodiment 12.1. The method of any one of Embodiments 1-11, wherein the one or more genes comprise all genes having a frequency of at least 30% in Table 2 or 3.

Embodiment 13. The method of any one of Embodiments 1-11, wherein the one or more genes comprise all genes having a frequency of at least 10%, 15%, 20%, 25%, 30%, 35%, 40%, 45%, 50%, 55%, 60%, 65%, 70%, 75%, or 80% in Table 2 or 3.

Embodiment 14. The method of any one of Embodiments 10-13, wherein the frequency of the gene is the number of runs in which the gene is selected in an elastic net modeling divided by the total number of runs of the elastic net modeling.

Embodiment 15. The method of any one of Embodiments 1-14, wherein the one or more genes comprise all genes in one of Tables 2-7, or all genes in Tables 8-9, or all genes in Tables 10-11.

Embodiment 15.1. The method of Embodiment 15, wherein the one or more genes comprise all genes in Table 2.

Embodiment 15.2. The method of Embodiment 15, wherein the one or more genes comprise all genes in Table 4.

Embodiment 15.3. The method of Embodiment 15, wherein the one or more genes comprise all genes in Table 5.

Embodiment 15.4. The method of Embodiment 15, wherein the one or more genes comprise all genes in Table 6.

Embodiment 15.5. The method of Embodiment 15, wherein the one or more genes comprise all genes in Table 7.

Embodiment 15.6. The method of Embodiment 15, wherein the one or more genes comprise all genes in Tables 8-9.

Embodiment 15.7. The method of Embodiment 15, wherein the one or more genes comprise all genes in Tables 10-11.

Embodiment 16. The method of Embodiment 15, wherein the one or more genes comprise all genes in Table 3.

Embodiment 17. The method of any one of Embodiments 1-16, wherein the one or more genes comprise two genes selected from one of combinations #1 to #55 of Table 13, three genes selected from one of combinations #56 to #220 of Table 13, four genes selected from one of combinations #221 to #550 of Table 13, or five genes selected from one of combinations #551 to #1012 of Table 13.

Embodiment 18. The method of any one of Embodiments 1-17, wherein the one or more genes comprise two genes selected from one of combinations #1 to #55 of Table 14, three genes selected from one of combinations #56 to #220 of Table 14, four genes selected from one of combinations #221 to #550 of Table 14, or five genes selected from one of combinations #551 to #1012 of Table 14.

Embodiment 19. The method of any one of Embodiments 1-18, wherein the one or more genes comprise at least one gene encoding a protein with adaptive immunity function selected from TAP1, MICB, HLA-B, HLA-C, HLA-E, HLA-H, and CTSS.

Embodiment 20. The method of any one of Embodiments 1-19, wherein the one or more genes comprise at least one gene encoding a protein with immune response function selected from CLEC2D, TNFRSF14, CD226, TNFAIP3, ANXA1, GSDMD, IKBKE, MAPK13, PARP9, TMEM9B, and UNC93B1.

Embodiment 21. The method of any one of Embodiments 1-20, wherein the one or more genes comprise at least one gene encoding a protein with cell adhesion/migration function selected from PPFIA4, LRFN5, ASTN1, MYL12A, CLDN5, CD9, SNED1, ITGA4, B4GALT1, and VWA1.

Embodiment 22. The method of any one of Embodiments 1-21, wherein the one or more genes comprise at least one gene encoding a protein with cellular RNA processing function selected from YBX2, EXOSC3, TAF1B, and USB1.

Embodiment 23. The method of any one of Embodiments 1-22, wherein the one or more genes comprise at least one gene encoding a protein with intracellular transportation function selected from SCG3, CPLX1, SCAMPI, SYTL2, CTAGE8, SLC52A1, HIP1, COPB1, and MYO5B.

Embodiment 24. The method of any one of Embodiments 1-23, wherein the one or more genes comprise at least one gene encoding a protein with DNA repair function selected from TYR, FAAP20, and FAM111A.

Embodiment 25. The method of any one of Embodiments 1-24, wherein the one or more genes comprise at least one gene encoding a protein with G-protein signaling function selected from GNAO1, PLCG2, ARHGEF35, ARHGEF16, RAPGEF3, DENND2D, and ADCY6.

Embodiment 26. The method of any one of Embodiments 1-25, wherein the one or more genes comprise at least one gene encoding a protein with neurotransmission function selected from SYN2, NSMF, CHRNA1, KCNT2, ATP2B2, TTYH2, CNRIP1, FAM155B, NTNG2, and AGRN.

Embodiment 27. The method of any one of Embodiments 1-26, wherein the one or more genes comprise at least one gene encoding a transcription factor selected from ETV7, HOXC11, SOX5, NHLH2, MYT1L, GRHL2, STAT6, DACH1, and ARID5A.

Embodiment 28. The method of any one of Embodiments 1-27, wherein the one or more genes comprise at least one gene encoding a protein with ubiquitination function selected from GID4, RNF112, and UBR1.

Embodiment 29. The method of any one of Embodiments 1-28, wherein the one or more genes comprise at least one gene selected from the group consisting of HLA-C, STAT6, TMCO4, CNRIP1, DCAF13, PRDM8, DACH1, and IKBKE.

Embodiment 30. The method of any one of Embodiments 1-28, wherein the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE.

Embodiment 31. The method of any one of Embodiments 1-28, wherein the one or more genes comprise at least one gene selected from the group consisting of HLA-C, STAT6, TMCO4, and CNRIP1.

Embodiment 32. The method of any one of Embodiments 1-28, wherein the one or more genes comprise 2, 3, or 4 genes selected from the group consisting of HLA-C, STAT6, TMCO4, and CNRIP1.

Embodiment 33. The method of any one of Embodiments 1-32, wherein the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7.

Embodiment 34. The method of any one of Embodiments 1-32, wherein the one or more genes comprise at least one gene selected from the group consisting of HLA-C, CNRIP1, JPH1, TMCO4, STAT6, DENND2D, and ETV7.

Embodiment 35. The method of any one of Embodiments 1-32, wherein the one or more genes comprise at least 2, 3, 4, 5, 6, or 7 genes selected from the group consisting of HLA-C, CNRIP1, JPH1, TMCO4, STAT6, DENND2D, and ETV7.

Embodiment 36. The method of any one of Embodiments 1-32, wherein the one or more genes comprise at least one gene selected from the group consisting of HLA-C, CNRIP1, JPH1, and TMCO4.

Embodiment 37. The method of any one of Embodiments 1-32, wherein the one or more genes comprise 2, 3, or 4 genes selected from the group consisting of HLA-C, CNRIP1, JPH1, and TMCO4.

Embodiment 38. The method of any one of Embodiments 1-37, wherein the one or more genes comprise HLA-C.

Embodiment 39. The method of any one of Embodiments 1-38, wherein the one or more genes do not comprise ANTXR1.

Embodiment 40. The method of any one of Embodiments 1-39, wherein the one or more genes do not comprise IFI35.

Embodiment 41. The method of any one of Embodiments 1-40, wherein the increased expression of the one or more upregulated genes in one of Tables 2-7, 8 and 10 is indicative of increased SVV sensitivity.

Embodiment 42. The method of claim 41, wherein the expression of the one or more upregulated genes is increased by at least 20%, at least 30%, at least 40%, at least 50%, at least 70%, at least 1-fold, at least 2-fold, at least 3-fold, at least 5-fold, or at least 10-fold, compared to a reference gene expression level.

Embodiment 43. The method of any one of Embodiments 1-42, wherein the reduced expression of the one or more downregulated genes in one of Tables 2-7, 9 and 11 is indicative of increased SVV sensitivity.

Embodiment 44. The method of claim 43, wherein the expression of the one or more downregulated genes is decreased by at least 10%, at least 20%, at least 30%, at least 40%, at least 50%, at least 60%, at least 70%, at least 80%, at least 90%, at least 95%, at least 98%, or at least 99%, compared to a reference gene expression level.

Embodiment 45. The method of Embodiment 42 or 44, wherein the reference gene expression level is a pre-determined value based on the expression level of the one or more genes in a non-cancerous cell, the expression level of the one or more genes in a reference set of non-cancerous samples, and/or the expression level of the one or more genes in a reference set of cancer samples with known sensitivity to SVV infection.

Embodiment 46. The method of any one of Embodiments 1-2, 4, 6, and 8-45, wherein the polynucleotide is a recombinant RNA molecule.

Embodiment 47. The method of any one of Embodiments 1-2, 4, 6, and 8-46, wherein the polynucleotide encoding the SVV viral genome is encapsulated in a particle.

Embodiment 48. The method of Embodiment 47, wherein the particle is a lipid nanoparticle.

Embodiment 49. The method of any one of Embodiments 1-48, wherein the expression level of the one or more genes is mRNA expression level.

Embodiment 50. The method of Embodiment 49, wherein determining the mRNA expression level comprises performing quantitative real time reverse transcriptase polymerase chain reaction (qRT-PCR), PCR, RNAseq, microarray, gene chip, nCounter Gene Expression Assay, Serial Analysis of Gene Expression (SAGE), Rapid Analysis of Gene Expression (RAGE), nuclease protection assays, Northern blotting, nucleic acid hybridization, or any other equivalent gene expression detection techniques.

Embodiment 51. The method of any one of Embodiments 1-48, wherein the expression level of the one or more genes is protein expression level.

Embodiment 52. The method of Embodiment 51, wherein the protein expression level is determined by antibody-based testing, immunoassay, radioimmunoassay (RIA), immunohistochemistry, immunofluorescence, chemiluminescence, phosphorescence, proteomics techniques, surface plasmon resonance (SPR), mass spectrometry, protein microarray, or any other equivalent protein expression detection techniques.

Embodiment 53. The method of any one of Embodiments 1-52, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, bladder cancer, and marginal zone lymphoma (MZL).

Embodiment 54. The method of any one of Embodiments 1-53, wherein the cancer is a neuroendocrine cancer.

Embodiment 55. The method of any one of Embodiments 1-54, wherein the cancer is selected from small cell lung cancer (SCLC), large cell neuroendocrine carcinoma (LCNEC), metastatic liver cancer, neuroendocrine-positive prostate cancer (e.g., treatment-emergent small-cell neuroendocrine prostate cancer (t-SCNC)), and Merkel cell carcinoma (MCC).

Embodiment 56. The method of any one of Embodiments 1-55, wherein the cancer is neuroendocrine-positive (NE+) metastatic castration-resistant prostate cancer (mCRPC).

Embodiment 57. The method of any one of Embodiments 1-55, wherein the cancer is small cell lung cancer (SCLC).

Embodiment 58. The method of Embodiment 57, wherein the cancer is NeuroD1+SCLC.

Embodiment 59. The method of any one of Embodiments 1-2, 4, 6, and 8-59, comprising administering a therapeutic agent selected from an immune checkpoint inhibitor, an engineered immune cell comprising an engineered antigen receptor, a JAK/STAT inhibitor, an mTOR inhibitor, an interferon (IFN) pathway inhibitor, and an HDAC inhibitor.

Embodiment 60. The method of Embodiment 59, wherein the immune checkpoint inhibitor is a PD-1 inhibitor or a PD-L1 inhibitor.

Embodiment 61. The method of any one of Embodiments 1-2, 5, 6, and 8-60, wherein the subject is a mouse, a rat, a rabbit, a cat, a dog, a horse, a non-human primate, or a human.

Embodiment 62. The method of any one of Embodiments 1-61, comprising obtaining a sample of the cancer for determining the expression level of the one or more genes in the cancer.

Embodiment 63. The method of any one of Embodiments 1-62, wherein a sample of the cancer is used for determining the expression level of the one or more genes, and wherein the sample is a formalin-fixed, paraffin-embedded (FFPE) sample, a fresh tissue sample, a frozen tissue sample, a tumor biopsy sample, an exosome, a wash fluid, a cell pellet, or a bodily fluid.

Embodiment 64. The method of any one of Embodiments 1-62, wherein a sample of the cancer is used for determining the expression level of the one or more genes, and wherein the sample comprises circulating tumor cells (CTCs) or cell-free RNA (cfRNA).

Embodiment 65. The method of any one of Embodiments 1-64, wherein the cancer has been treated with one or more therapeutic agents.

Embodiment 66. The method of Embodiment 65, wherein the cancer has relapsed after the treatment of the therapeutic agent.

Embodiment 67. The method of Embodiment 65 or 66, wherein the therapeutic agent is a chemotherapeutic agent, a checkpoint kinase inhibitor, or a PARP inhibitor.

Embodiment 68. The method of Embodiment 65 or 66, wherein the therapeutic agent is a platinum-based drug.

Embodiment 69. The method of Embodiment 65 or 66, wherein the therapeutic agent is Cisplatin.

Embodiment 70. A kit, comprising reagents for determining the expression level of one or more genes in a sample from a subject in need of, wherein the one or more genes comprise any one of the genes listed in one of Tables 1-14 or a combination thereof.

Embodiment 71. The kit of Embodiment 70, wherein the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 genes selected from one of Tables 1-14.

Embodiment 72. The kit of Embodiment 70 or 71, wherein the one or more genes have a frequency of at least 5%, 10%, 15%, 20%, 25%, 30%, 35%, 40%, 45%, 50%, 55%, 60%, 65%, 70%, 75%, or 80% in Table 2 or 3.

Embodiment 73. The kit of any one of Embodiments 70-72, wherein the one or more genes comprise all genes having a frequency of at least 40% in Table 2 or 3.

Embodiment 74. The kit of any one of Embodiments 70-73, wherein the one or more genes comprise all genes having a frequency of at least 10%, 15%, 20%, 25%, 30%, 35%, 40%, 45%, 50%, 55%, 60%, 65%, 70%, 75%, or 80% in Table 2 or 3.

Embodiment 75. The kit of any one of Embodiments 70-74, wherein the frequency of the gene is the number of runs in which the gene is selected in an elastic net modeling divided by the total number of runs of the elastic net modeling.

Embodiment 76. The kit of any one of Embodiments 70-75, wherein the one or more genes comprise all genes in one of Tables 2-7, or all genes in Tables 8-9, or all genes in Tables 10-11.

Embodiment 76.1. The kit of Embodiment 76, wherein the one or more genes comprise all genes in Table 2.

Embodiment 76.2. The kit of Embodiment 76, wherein the one or more genes comprise all genes in Table 4.

Embodiment 76.3. The kit of Embodiment 76, wherein the one or more genes comprise all genes in Table 5.

Embodiment 76.4. The kit of Embodiment 76, wherein the one or more genes comprise all genes in Table 6.

Embodiment 76.5. The kit of Embodiment 76, wherein the one or more genes comprise all genes in Table 7.

Embodiment 76.6. The kit of Embodiment 76, wherein the one or more genes comprise all genes in Tables 8-9.

Embodiment 77. The kit of Embodiment 76, wherein the one or more genes comprise all genes in Table 3.

Embodiment 78. The kit of any one of Embodiments 70-77, wherein the one or more genes comprise two genes selected from one of combinations #1 to #55 of Table 13, three genes selected from one of combinations #56 to #220 of Table 13, four genes selected from one of combinations #221 to #550 of Table 13, or five genes selected from one of combinations #551 to #1012 of Table 13.

Embodiment 79. The kit of any one of Embodiments 70-78, wherein the one or more genes comprise two genes selected from one of combinations #1 to #55 of Table 14, three genes selected from one of combinations #56 to #220 of Table 14, four genes selected from one of combinations #221 to #550 of Table 14, or five genes selected from one of combinations #551 to #1012 of Table 14.

Embodiment 80. The kit of any one of Embodiments 70-79, wherein the one or more genes comprise at least one gene encoding a protein with adaptive immunity function selected from TAP1, MICB, HLA-B, HLA-C, HLA-E, HLA-H, and CTSS.

Embodiment 81. The kit of any one of Embodiments 70-80, wherein the one or more genes comprise at least one gene encoding a protein with immune response function selected from CLEC2D, TNFRSF14, CD226, TNFAIP3, ANXA1, GSDMD, IKBKE, MAPK13, PARP9, TMEM9B, and UNC93B1.

Embodiment 82. The kit of any one of Embodiments 70-81, wherein the one or more genes comprise at least one gene encoding a protein with cell adhesion/migration function selected from PPFIA4, LRFN5, ASTN1, MYL12A, CLDN5, CD9, SNED1, ITGA4, B4GALT1, and VWA1.

Embodiment 83. The kit of any one of Embodiments 70-82, wherein the one or more genes comprise at least one gene encoding a protein with cellular RNA processing function selected from YBX2, EXOSC3, TAF1B, and USB1.

Embodiment 84. The kit of any one of Embodiments 70-83, wherein the one or more genes comprise at least one gene encoding a protein with intracellular transportation function selected from SCG3, CPLX1, SCAMPI, SYTL2, CTAGE8, SLC52A1, HIP1, COPB1, and MYO5B.

Embodiment 85. The kit of any one of Embodiments 70-84, wherein the one or more genes comprise at least one gene encoding a protein with DNA repair function selected from TYR, FAAP20, and FAM111A.

Embodiment 86. The kit of any one of Embodiments 70-85, wherein the one or more genes comprise at least one gene encoding a protein with G-protein signaling function selected from GNAO1, PLCG2, ARHGEF35, ARHGEF16, RAPGEF3, DENND2D, and ADCY6.

Embodiment 87. The kit of any one of Embodiments 70-86, wherein the one or more genes comprise at least one gene encoding a protein with neurotransmission function selected from SYN2, NSMF, CHRNA1, KCNT2, ATP2B2, TTYH2, CNRIP1, FAM155B, NTNG2, and AGRN.

Embodiment 88. The kit of any one of Embodiments 70-87, wherein the one or more genes comprise at least one gene encoding a transcription factor selected from ETV7, HOXC11, SOX5, NHLH2, MYT1L, GRHL2, STAT6, DACH1, and ARID5A.

Embodiment 89. The kit of any one of Embodiments 70-88, wherein the one or more genes comprise at least one gene encoding a protein with ubiquitination function selected from GID4, RNF112, and UBR1.

Embodiment 90. The kit of any one of Embodiments 70-89, wherein the one or more genes comprise at least one gene selected from the group consisting of HLA-C, STAT6, TMCO4, CNRIP1, DCAF13, PRDM8, DACH1, and IKBKE.

Embodiment 91. The kit of any one of Embodiments 70-90, wherein the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE.

Embodiment 92. The kit of any one of Embodiments 70-91, wherein the one or more genes comprise at least one gene selected from the group consisting of HLA-C, STAT6, TMCO4, and CNRIP1.

Embodiment 93. The kit of any one of Embodiments 70-91, wherein the one or more genes comprise 2, 3, or 4 genes selected from the group consisting of HLA-C, STAT6, TMCO4, and CNRIP1.

Embodiment 94. The kit of any one of Embodiments 70-93, wherein the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7.

Embodiment 95. The kit of any one of Embodiments 70-94, wherein the one or more genes comprise at least one gene selected from the group consisting of HLA-C, CNRIP1, JPH1, TMCO4, STAT6, DENND2D, and ETV7.

Embodiment 96. The kit of any one of Embodiments 70-95, wherein the one or more genes comprise at least 2, 3, 4, 5, 6, or 7 genes selected from the group consisting of HLA-C, CNRIP1, JPH1, TMCO4, STAT6, DENND2D, and ETV7.

Embodiment 97. The kit of any one of Embodiments 70-96, wherein the one or more genes comprise at least one gene selected from the group consisting of HLA-C, CNRIP1, JPH1, and TMCO4.

Embodiment 98. The kit of any one of Embodiments 70-96, wherein the one or more genes comprise 2, 3, or 4 genes selected from the group consisting of HLA-C, CNRIP1, JPH1, and TMCO4.

Embodiment 99. The kit of any one of Embodiments 70-98, wherein the one or more genes comprise HLA-C.

Embodiment 100. The kit of any one of Embodiments 70-99, wherein the one or more genes do not comprise ANTXR1.

Embodiment 101. The kit of any one of Embodiments 70-100, wherein the one or more genes do not comprise IFI35.

Embodiment 102. The kit of any one of Embodiments 70-101, wherein the kit comprises the reagents for determining the mRNA expression level of the one or more genes.

Embodiment 103. The kit of Embodiment 102, wherein the reagents comprises reagents for performing quantitative real time reverse transcriptase polymerase chain reaction (qRT-PCR), PCR, RNAseq, microarray, gene chip, nCounter Gene Expression Assay, Serial Analysis of Gene Expression (SAGE), Rapid Analysis of Gene Expression (RAGE), nuclease protection assays, Northern blotting, nucleic acid hybridization, or any other equivalent gene expression detection techniques.

Embodiment 104. The kit of any one of Embodiments 70-103, wherein the kit comprises the reagents for determining the protein expression level of the one or more genes.

Embodiment 105. The kit of Embodiment 104, wherein the reagents comprises reagents for performing antibody-based testing, immunoassay, radioimmunoassay (RIA), immunohistochemistry, immunofluorescence, chemiluminescence, phosphorescence, proteomics techniques, surface plasmon resonance (SPR), mass spectrometry, protein microarray, or any other equivalent protein expression detection techniques.

Embodiment 106. The kit of any one of Embodiments 70-105, wherein the sample is a formalin-fixed, paraffin-embedded (FFPE) sample, a fresh tissue sample, a frozen tissue sample, a tumor biopsy sample, an exosome, a wash fluid, a cell pellet, a bodily fluid, a circulating tumor cells (CTCs) sample, or a cell-free RNA (cfRNA) sample.

Embodiment 107. The kit of any one of Embodiments 70-106, wherein the sample is a cancer sample and wherein the kit is for valuating the sensitivity of the cancer to SVV infection.

Embodiment 108. The kit of any one of Embodiments 70-107, wherein the kit is for use in combination with a composition comprising a Seneca Valley Virus (SVV) or a polynucleotide encoding the SVV viral genome for treating a cancer in the subject.

Embodiment 109. The kit of Embodiment 107 or 108, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, diffuse large B-cell lymphoma (DLBCL), sarcoma, a neuroblastoma, a neuroendocrine cancer, a rhabdomyosarcoma, a medulloblastoma, bladder cancer, and marginal zone lymphoma (MZL).

Embodiment 110. The kit of any one of Embodiments 107-109, wherein the cancer is a neuroendocrine cancer.

Embodiment 111. The kit of any one of Embodiments 107-110, wherein the cancer is selected from small cell lung cancer (SCLC), large cell neuroendocrine carcinoma (LCNEC), metastatic liver cancer, neuroendocrine-positive prostate cancer (e.g., treatment-emergent small-cell neuroendocrine prostate cancer (t-SCNC)), and Merkel cell carcinoma (MCC).

Embodiment 112. The kit of any one of Embodiments 107-111, wherein the cancer is neuroendocrine-positive (NE+) metastatic castration-resistant prostate cancer (mCRPC).

Embodiment 113. The kit of any one of Embodiments 107-112, wherein the cancer is small cell lung cancer (SCLC).

Embodiment 114. The kit of any one of Embodiments 107-113, wherein the cancer is NeuroD1+SCLC.

Embodiment 115. Use of the kit of any one of Embodiments 107-114 for classifying sensitivity of the cancer in the subject to a Seneca Valley Virus (SVV).

### EXAMPLES

The following examples are given for the purpose of illustrating various embodiments of the disclosure and are not meant to limit the present disclosure in any fashion. The present examples, along with the methods described herein are presently representative of preferred embodiments, are exemplary and are not intended as limitations on the scope of the disclosure. Changes therein and other uses which are encompassed within the spirit of the disclosure as defined by the scope of the claims will occur to those skilled in the art.

### Example 1: Development of a Gene Signature Panel to Predict SVV-Sensitivity for SCLC Using Elastic Net Search

A gene signature panel was developed to predict small cell lung cancer (SCLC) sensitivity to SVV infection based on a training data set comprising the RNA-seq data and SVV sensitivity information of 20 SCLC cell lines from Cancer Cell Line Encyclopedia (CCLE; information available at the depmap website) and 14 SCLC patient-derived xenograft (PDX) lines, as shown in Table 15 below.

TABLE 15
SVV Cell Line Training Set for
ELN28 and ELN28_reduced Panels.

| Cell Line | Source | Subtype | SVV Sensitivity |
|---|---|---|---|
| LUS188 | PDX | | S |
| LUS184 | PDX | | S |
| LUS203 | PDX | | S |
| LUS207 | PDX | | S |
| LUS171 | PDX | | S |
| LUS231 | PDX | | S |
| LUS180 | PDX | | S |
| LUS139 | PDX | | R |
| LUS256 | PDX | | R |
| LUS178 | PDX | | R |
| LUS266 | PDX | | R |
| LUS159 | PDX | | R |
| LUS242 | PDX | | R |
| LUS377 | PDX | | R |
| DMS144 | CCLE | SCLC-YAP1 | R |
| SW1271 | CCLE | SCLC-YAP1 | R |
| CORL311 | CCLE | SCLC-POU2F3 | R |
| NCIH1048 | CCLE | SCLC-POU2F3 | R |
| NCIH211 | CCLE | SCLC-POU2F3 | R |
| COLO668 | CCLE | SCLC-ASCL1 | R |
| DMS53 | CCLE | SCLC-ASCL1 | R |
| DMS79 | CCLE | SCLC-ASCL1 | R |
| NCIH2196 | CCLE | SCLC-ASCL1 | R |
| NCIH1694 | CCLE | SCLC-NEUROD1 | S |
| NCIH2171 | CCLE | SCLC-NEUROD1 | S |
| NCIH446 | CCLE | SCLC-NEUROD1 | S |
| NCIH524 | CCLE | SCLC-NEUROD1 | S |
| NCIH82 | CCLE | SCLC-NEUROD1 | S |
| NCIH1184 | CCLE | SCLC-ASCL1 | S |
| NCIH1963 | CCLE | SCLC-ASCL1 | S |
| NCIH2029 | CCLE | SCLC-ASCL1 | S |
| NCIH209 | CCLE | SCLC-ASCL1 | S |
| NCIH889 | CCLE | SCLC-ASCL1 | S |
| SHP77 | CCLE | SCLC-ASCL1 | S |

(S = Sensitive, R = Resistant)

The RNA-seq data was analyzed using an elastic net (ELN) search to identify genes with predictive power for classifying samples into SVV-sensitive (S) or SVV-resistant (R). The ELN search (Zhou and Hastie, Journal of the Royal Statistical Society, vol B 67, pg 301, 2005) was run 1000 times on the training set, each time with an 80% random selection of the 28 lines. This ELN search identified a set of 30 genes that were upregulated and 43 genes that were downregulated in SVV sensitive lines in at least 5% of the model runs (Table 16 below). The frequency refers to the number of times the gene was selected in a model out of 1,000 model runs. This gene panel is herein referred to as the ELN28 gene panel.

TABLE 16
Up and Down regulated Genes Identified in ELN28

| | |
|---|---|
| Upregulated Genes | Downregulated genes |

| | Frequency | | Frequency |
|---|---|---|---|
| Gene symbol | (%) | Gene Symbol | (%) |
| RPL23AP94 | 45.6 | TAP1 | 67.6 |
| SYN2 | 36.0 | PLCG2 | 50.9 |
| NSMF | 30.9 | ARHGEF35 | 45.8 |
| PPFIA4 | 28.3 | ARHGEF16 | 42.7 |
| SELENOO | 26.5 | MICB | 38.6 |
| CHRNA1 | 23.1 | TMCO4 | 36.0 |
| GNAO1 | 20.9 | RAPGEF3 | 33.5 |
| TMEM249 | 19.3 | NPC2 | 30.5 |
| SCG3 | 17.6 | MYL12A | 26.1 |
| CCNJL | 16.3 | ANO7L1 | 25.9 |
| JPH1 | 14.7 | TNFRSF10B | 23.6 |
| LRFN5 | 12.8 | HLA-B | 20.6 |
| CPLX1 | 12.5 | PROM2 | 19.0 |
| SCAMP1 | 12.0 | USP43 | 18.4 |
| ASTN1 | 9.7 | RHBDF1 | 17.0 |
| TRBVB | 9.4 | HLA-C | 15.9 |
| KCNT2 | 8.6 | SYTL2 | 15.9 |
| ATP2B2 | 8.1 | ETV7 | 15.7 |
| CYP7B1 | 7.6 | DENND2D | 15.1 |
| PPP1R17 | 6.9 | HOXC11 | 13.8 |
| CENPV | 6.4 | CLEC2D | 13.6 |
| CCDC157 | 6.0 | ARHGEF34P | 13.5 |
| SOX5 | 5.7 | TNFRSF14 | 12.8 |
| BCRP2 | 5.6 | CD226 | 12.5 |
| FAM118A | 5.6 | NBPF14 | 11.9 |
| TTYH2 | 5.4 | PSMB9 | 11.4 |
| ANKRD20A8P | 5.2 | CD9 | 9.9 |
| CLDN5 | 5.2 | ACBD4 | 8.6 |
| NHLH2 | 5.2 | HLA-E | 8.3 |
| MYT1L | 5.1 | TNFAIP3 | 7.5 |
| | | GRHL2 | 7.2 |
| | | ANXA1 | 7.1 |
| | | CTAGE8 | 7.0 |
| | | IRS4 | 6.9 |
| | | TMED11P | 6.3 |
| | | MPIG6B | 5.7 |
| | | VWA5A | 5.6 |
| | | ERP27 | 5.3 |
| | | PRSS22 | 5.3 |
| | | CTSS | 5.2 |
| | | YBX2 | 5.2 |
| | | STAT6 | 5.1 |
| | | PLPP2 | 5.0 |

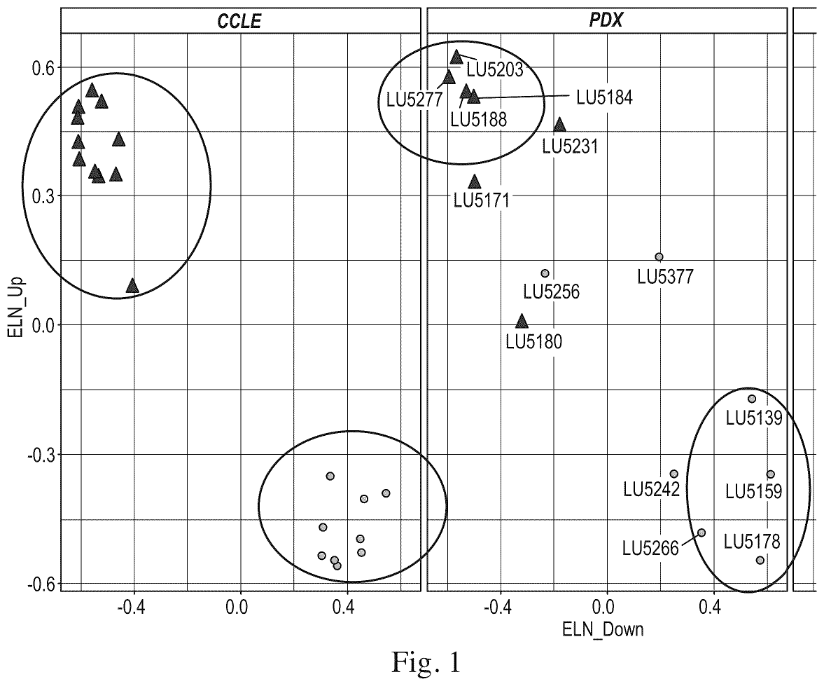

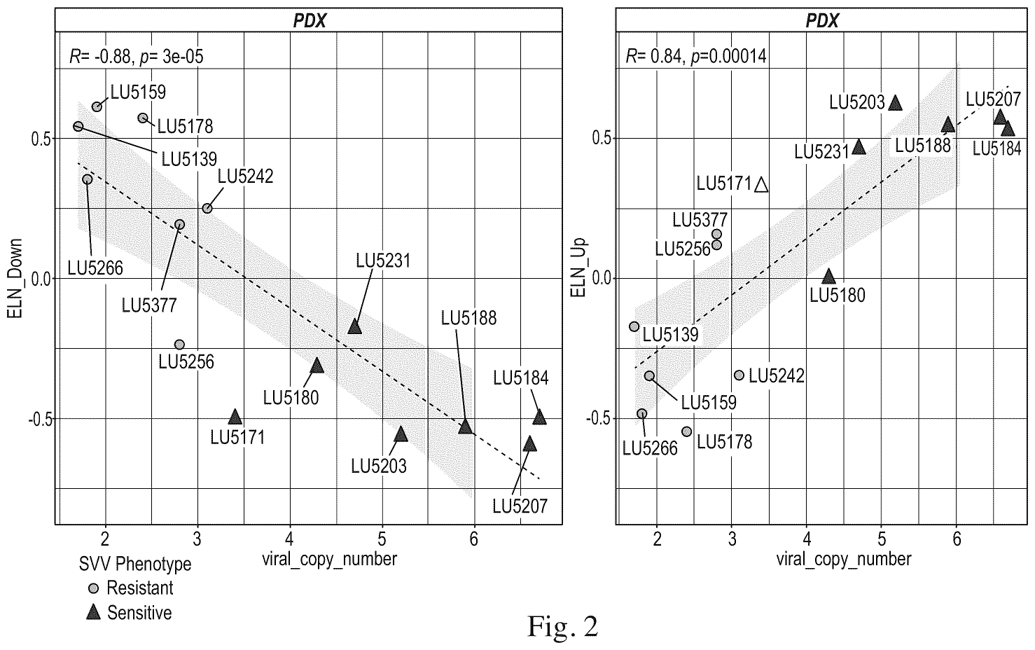

FIG. 1 shows the results of the Gene Set Variation Analysis (GSVA) run (Hanzelmann et al., BMC Bioinformatics. 2013 Jan. 16; 14:7) based on the ELN28 gene panel, which successfully grouped the SVV-sensitive cell lines at the upper left corner of the chart and the SVV-resistant cell lines at the lower right corner of the chart. Notably, the three PDX lines that fell in the middle of the chart (LU5180, LU5256, & LU5377) showed only intermediate SVV viral loads in SVV infection experiments, suggesting that the ELN28 gene panel provides predictive power to differentiate cell lines that are only moderately sensitive to SVV from those that are highly sensitive to SVV. Indeed, as shown in FIG. 2, plotting the SVV viral titers for the PDX lines based on the up-regulated and down-regulated ELN28 panel showed a strong correlation between the levels of viral titer in the PDX lines and the expression levels of the genes.

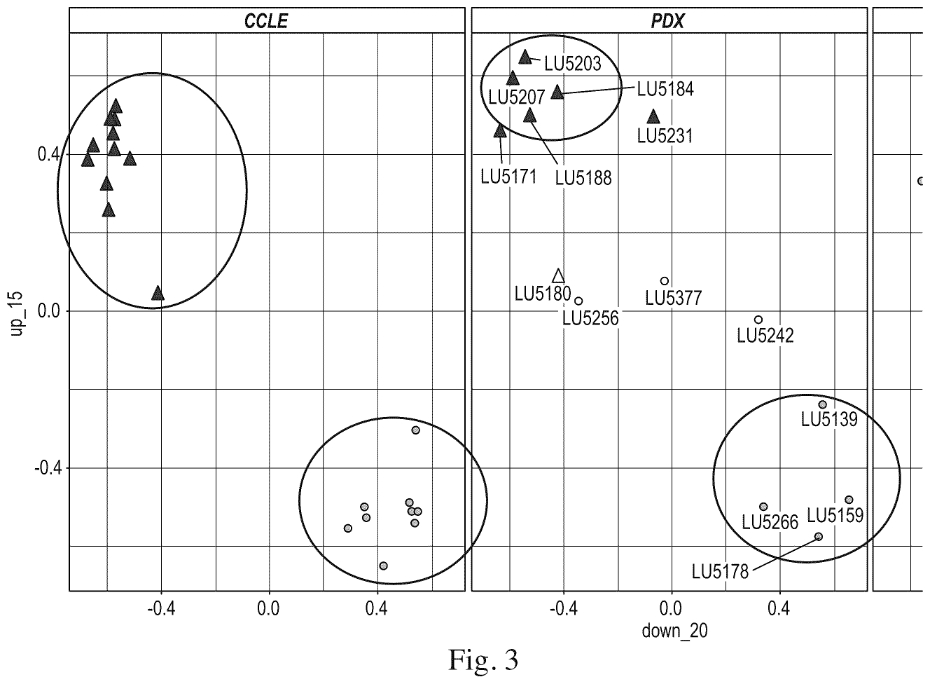

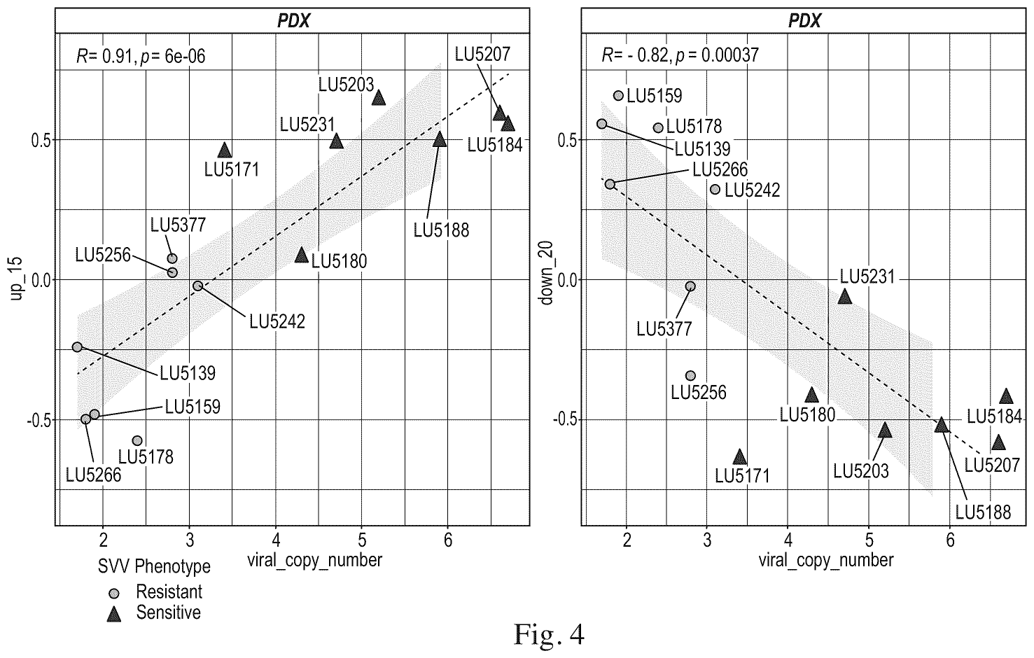

A reduced gene panel was then generated by sequentially removing the genes in the ELN28 gene panel, starting from the lowest frequency genes in Table 16. At each iteration, the quality of the resulting model was assessed by computing the Spearman correlation to viral load for the GSVA-derived scores as in FIG. 1. The resulting gene signature panel, termed ELN28_reduced, comprises 15 up-regulated genes and 20 down-regulated genes (Table 17 below). FIG. 3 (gene signature scores) and FIG. 4 (viral copy number) show that the ELN28_reduced panel performed similarly to the ELN28 panel.

TABLE 17
Up- and Down-regulated Genes Identified
in ELN28_reduced Panel

| | |
|---|---|
| Upregulated Genes | Downregulated genes |

| | Frequency | | Frequency |
|---|---|---|---|
| Gene symbol | (%) | Gene Symbol | (%) |
| RPL23AP94 | 45.6 | TAP1 | 67.6 |
| SYN2 | 36.0 | PLCG2 | 50.9 |
| NSMF | 30.9 | ARHGEF35 | 45.8 |
| PPFIA4 | 28.3 | ARHGEF16 | 42.7 |
| SELENOO | 26.5 | MICB | 38.6 |
| CHRNA1 | 23.1 | TMCO4 | 36.0 |
| GNAO1 | 20.9 | RAPGEF3 | 33.5 |
| TMEM249 | 19.3 | NPC2 | 30.5 |
| SCG3 | 17.6 | MYL12A | 26.1 |
| CCNJL | 16.3 | ANO7L1 | 25.9 |
| JPH1 | 14.7 | TNFRSF10B | 23.6 |
| LRFN5 | 12.8 | HLA-B | 20.6 |
| CPLX1 | 12.5 | PROM2 | 19.0 |
| SCAMP1 | 12.0 | USP43 | 18.4 |
| ASTN1 | 9.7 | RHBDF1 | 17.0 |
| | | HLA-C | 15.9 |
| | | SYTL2 | 15.9 |
| | | ETV7 | 15.7 |
| | | DENND2D | 15.1 |
| | | HOXC11 | 13.8 |

### Example 2: Construction of Alternative ELN Gene Signature Panels

An alternative ELN-based gene signature panel was developed based on a training set comprising the RNA-seq data and SVV sensitivity information of 17 SCLC cell lines from Cancer Cell Line Encyclopedia (CCLE; depmap.org/portal/), 8 SCLC patient derived xenograft (PDX) lines, and 6 cell lines derived from H1299, following the protocol used in Example 1. Table 18A below lists detailed information of the cell lines used in the training.

TABLE 18A
SVV Cell Line Training Set for ELN_1 Model.

| Cell Line | Source | SVV Sensitivity |
|---|---|---|
| COLO668 | CCLE | R |
| CORL311 | CCLE | R |
| DMS114 | CCLE | R |
| DMS153 | CCLE | R |
| DMS53 | CCLE | R |
| DMS79 | CCLE | R |
| H12R_CG | H1299 | R |
| H12R_JB1 | H1299 | R |
| H12R_JB2 | H1299 | R |
| LU5139 | PDX | R |
| LU5159 | PDX | R |
| LU5178 | PDX | R |
| LU5266 | PDX | R |
| NCIH1048 | CCLE | R |
| NCIH211 | CCLE | R |
| NCIH2196 | CCLE | R |
| NCIH526 | CCLE | R |
| SW1271 | CCLE | R |
| 5JB | H1299 | S |
| H12 | H1299 | S |
| H1299 | H1299 | S |
| LU5184 | PDX | S |
| LU5188 | PDX | S |
| LU5203 | PDX | S |
| LU5207 | PDX | S |
| NCIH1299 | CCLE | S |
| NCIH1694 | CCLE | S |
| NCIH2171 | CCLE | S |
| NCIH446 | CCLE | S |
| NCIH82 | CCLE | S |
| SHP77 | CCLE | S |

(S = Sensitive, R = Resistant)

This ELN search identified a set of 22 genes that are upregulated and 26 genes that are downregulated in SVV sensitive lines (Table 18B below). Genes in either column of the table are ordered according to their frequency values in the ELN modeling, with the genes having the highest frequency appearing at the top of the columns. This panel is herein referred to as the ELN_1 gene signature panel.

TABLE 18B
Up- and down-regulated Genes Identified
in ELN_1 Gene Signature Panel

| Upregulated Genes | Downregulated genes |
|---|---|
| PRDM8 | HLA-C |
| CNRIP1 | PARP9 |
| MCC | DENND2D |
| JPH1 | ETV7 |
| TAF1B | HLA-H |
| GLCE | TMEM9B |
| MIEF1 | STAT6 |
| DACH1 | PDCL3P4 |
| USB1 | STAT1 |
| TTYH2 | NUCB2 |
| ITGA4 | COPB1 |
| PPP4R4 | SMAD7 |
| RIPPLY2 | CEP295 |
| RNF112 | FAM111A |
| EXOSC3 | UNC93B1 |
| NTNG2 | TMCO4 |
| PMPCA | MBOAT7 |
| HIP1 | PSMB8 |
| RPS7P1 | B4GALT1 |
| GID4 | IKBKE |
| UBR1 | EPS8L2 |
| DCAF13 | TMEM80 |
| | MYO5B |
| | DNAJC16 |
| | GSDMD |
| | MAPK13 |

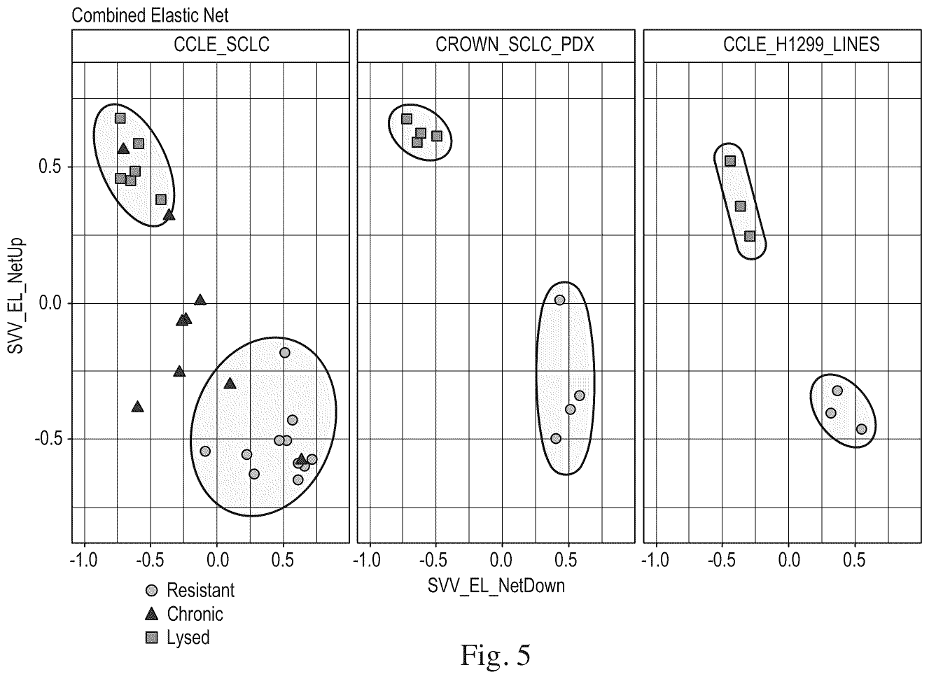

FIG. 5 shows the results of the GSVA algorithm run based on the ELN_1 gene signature panel, which successfully grouped the SVV-sensitive cell lines at the upper left corner of the chart and the SVV-resistant cell lines at the lower right corner of the chart. Notably, for most of the CCLE cell lines that can be chronically infected with SVV but not lysed by SVV, they display intermediate gene signature scores in the chart, suggesting that the gene panel provides predictive power to differentiate cell lines that are only moderately sensitive to SVV from those that are highly sensitive to SVV.

Additional ELN-based gene signature panels were developed based on alternative training sets following the same procedures. Specifically, the RNA-seq data and SVV sensitivity information of cell lines listed in Table 19 below were used to form the gene panel of Table 5 (herein referred to as the ELN_C gene signature panel), and the RNA-seq data and SVV sensitivity information of cell lines listed in Table 20 below were used to form the gene panel of Table 6 (herein referred to as the ELN_2 gene signature panel) and the gene panel of Table 7 (herein referred to as the ELN_3 gene signature panel). Genes in either column of the tables are ordered according to their frequency values in the ELN modeling, with the genes having the highest frequency appearing at the top of the columns.

TABLE 19
SVV Cell Line Training Set for ELN_C Model.

| Cell Line | Source | SVV Sensitivity |
|---|---|---|
| COLO668 | CCLE | R |
| CORL311 | CCLE | R |
| DMS114 | CCLE | R |
| DMS153 | CCLE | R |
| DMS53 | CCLE | R |
| DMS79 | CCLE | R |
| NCIH1048 | CCLE | R |
| NCIH211 | CCLE | R |
| NCIH2196 | CCLE | R |
| NCIH526 | CCLE | R |
| SW1271 | CCLE | R |
| LU5139 | PDX | R |
| LU5159 | PDX | R |
| LU5178 | PDX | R |
| LU5266 | PDX | R |
| H12R_JB1 | H1299 | R |
| H12R_CG | H1299 | R |
| H12R_JB2 | H1299 | R |
| NCIH1184 | CCLE | MS |
| NCIH146 | CCLE | MS |
| NCIH1618 | CCLE | MS |
| NCIH1963 | CCLE | MS |
| NCIH2029 | CCLE | MS |
| NCIH209 | CCLE | MS |
| NCIH524 | CCLE | MS |
| NCIH69 | CCLE | MS |
| NCIH889 | CCLE | MS |
| NCIH1299 | CCLE | S |
| NCIH1694 | CCLE | S |
| NCIH2171 | CCLE | S |
| NCIH446 | CCLE | S |
| NCIH82 | CCLE | S |
| SHP77 | CCLE | S |
| LU5184 | PDX | S |
| LU5188 | PDX | S |
| LU5203 | PDX | S |
| LU5207 | PDX | S |
| 5JB | H1299 | S |
| H1299 | H1299 | S |
| H12 | H1299 | S |

(S = Sensitive, MS = Moderately Sensitive, R = Resistant)

TABLE 20
SVV Cell Line Training Set for ELN_2 and ELN_3 Models.

| Cell Line | Source | SVV Sensitivity |
|---|---|---|
| LUS139 | PDX | R |
| LUS256 | PDX | R |
| LUS178 | PDX | R |
| LUS266 | PDX | R |
| LUS159 | PDX | R |
| LUS242 | PDX | R |
| LUS377 | PDX | R |
| DMS144 | CCLE | R |
| SW1271 | CCLE | R |
| CORL311 | CCLE | R |
| NCIH1048 | CCLE | R |
| NCIH211 | CCLE | R |
| COLO668 | CCLE | R |
| DMS53 | CCLE | R |
| DMS79 | CCLE | R |
| NCIH2196 | CCLE | R |
| H12R_JB1 | | R |
| H12R_CG | | R |
| H12R_JB2 | | R |
| LUS188 | PDX | S |
| LUS184 | PDX | S |
| LUS203 | PDX | S |
| LUS207 | PDX | S |
| LUS171 | PDX | S |
| LUS231 | PDX | S |
| LUS180 | PDX | S |
| NCIH1694 | CCLE | S |
| NCIH2171 | CCLE | S |
| NCIH446 | CCLE | S |
| NCIH524 | CCLE | S |
| NCIH82 | CCLE | S |
| NCIH1184 | CCLE | S |
| NCIH1963 | CCLE | S |
| NCIH2029 | CCLE | S |
| NCIH209 | CCLE | S |
| NCIH889 | CCLE | S |
| SHP77 | CCLE | S |
| H12S_5JB | | S |
| H12S_CG | | S |
| H12S_H12 | | S |

(S = Sensitive, R = Resistant)

### Example 3: Prediction of SVV-Sensitive SCLC Based on the Gene Signature Panel

The ELN_1 gene signature was applied to the RNA seq data of human small cell lung cancer (SCLC) patients from different databases to predict the percentage of SVV-sensitive cancers in these patients. According to Rudin et al., Nat Rev Cancer. 2019 May; 19(5):289-297, small cell lung carcinoma (SCLC) can be classified into four subtypes based on the RNA expression of ASCL1, NEUROD1, POU2F3, and YAP1 transcriptional regulators. According to the ELN_1 gene signature panel, the NeuroD1+SCLC subtype is predicted to be SVV sensitive (Table 21 below), suggesting that the NeuroD1+SCLC subtype is particularly suitable for SVV treatment.

TABLE 21
Prediction of SVV Sensitivity for NE-SCLC

SCLC
Neuroendocrine (NE)
SCLC subtype

| | ASCL1 | NeuroD1 |
|---|---|---|
| % Total Patients | 70 | 11 |
| % Predicted to be SVV sensitive | 37 | 90 |

### Example 4: Elastic Net (ELN) Based SVV Gene Signature Panel is Applicable to Predicting SVV Sensitivity Based on Circulating Tumor Cells (CTC)

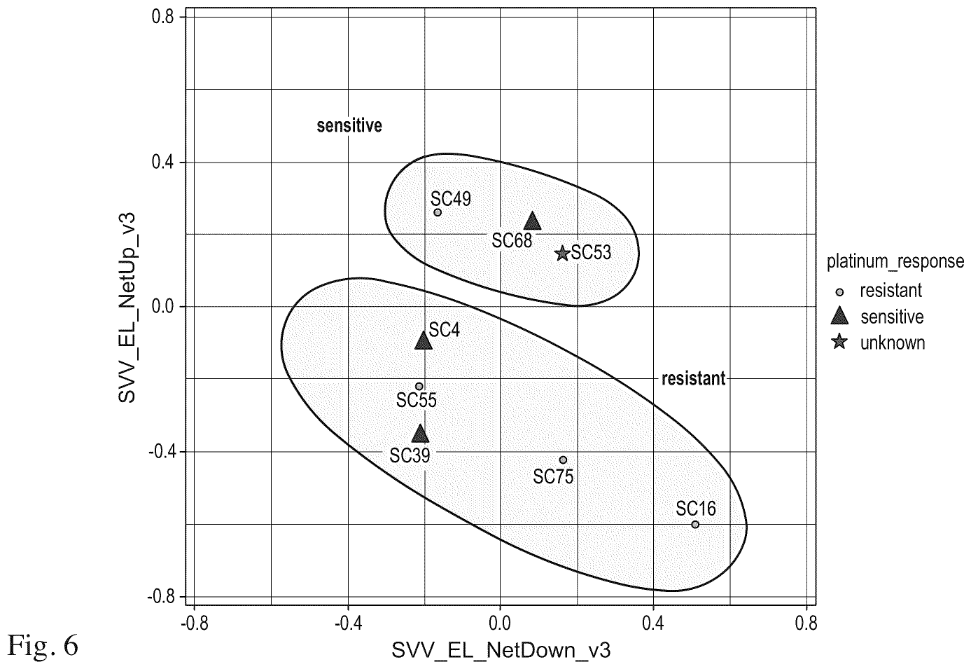

The ELN_3 gene signature panel was used to predict SVV sensitivity of eight different circulating tumor cell samples (CTC) derived from SCLC xenograft models (CDX models) based on data in Stewart et al., Nature Cancer. vol 1 (2020) 423-436. One CDX model, SC49, is NeuroD1+ subtype. The other 7 CDX models are ASCL1+ subtype. The averaged single cell RNA-seq (scRNA-seq) data for each line (provided in GSE138474, available at the NCBI Gene Expression Omnibus website) was used for prediction of SVV sensitivity. As shown in FIG. 6, the NeuroD1+ line (SC49) and two ASCL1+ lines (SC53 and SC68) were predicted to be SVV sensitive, whereas the other five CDX models were predicted to be SVV resistant.

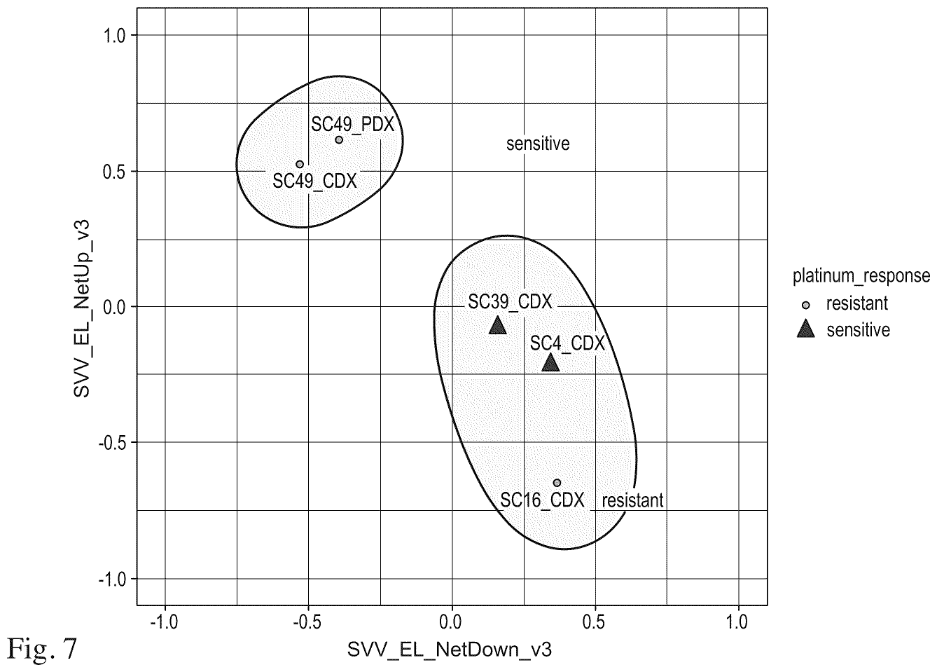

Notably, the ELN_3 gene signature analysis produced very similar results for samples derived from two different sources: CTC and tumor biopsies. As shown in FIG. 7, the resulting signature scores for NeuroD1+ line SC49 are very similar for CTC and tumor biopsy samples.

### Example 5. SVV Gene Signature Panel Predicts Increased SVV Sensitivity of SCLC after Relapse from Prior Treatment

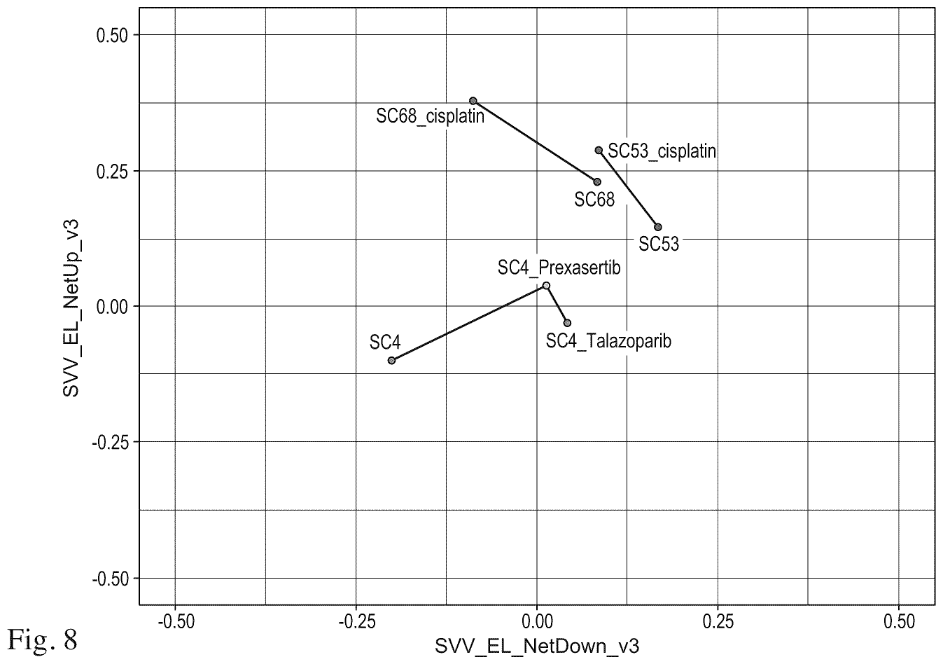

The ELN_3 gene signature panel was used to predict whether drug treatment increases the tumor's SVV sensitivity after relapse. Three CDX models described in the last Example have RNA-seq data from tumors prior to treatment and after treatment relapse. The treatments include cisplatin, prexasertib, or talazoparib. As shown in FIG. 8, relapsed SCLC showed further increased overall expression of the neuroendocrine signature genes (in the group of up-regulated genes) of the corresponding tumor. In addition, cisplatin treatment resulted in further decreased overall expression of the immune signature genes (in the group of down-regulated genes) of the corresponding tumor. Therefore, relapsed SCLC (especially cisplatin treated SCLC) are predicted to have increased SVV sensitivity and would therefore be more susceptible to SVV treatment.

### Example 6: Construction of an Alternative Gene Signature Panel

A subset of cell lines that showed the highest level of infectivity in SCLC were combined with SVV-resistant cell lines according to Table 22 below to build a model based on genes that are differentially expressed between the resistant and sensitive samples while accounting for the variations between cell lines and PDX samples.

TABLE 22
SVV Cell Line Training Set for SVV100 Model.

| Cell Line | SVV Sensitivity |
|---|---|
| 5637 | R |
| 786O | R |
| A375 | R |
| A498 | R |
| A549 | R |
| ACHN | R |
| AsPC1 | S |
| BT549 | R |
| BxPC3 | R |
| Caki1 | R |
| COLO 205 | R |
| D283Med | S |
| Daoy | R |
| DMS114 | R |
| DMS153 | S |
| DMS53 | R |
| DMS79 | R |
| DU145 | R |
| EKVX | R |
| HCC33 | S |
| HCT116 | R |
| HCT15 | R |
| HeLa | R |
| Hep3B217 | R |
| HepG2 | R |
| HL60 | R |
| HOP62 | R |
| HOP92 | R |
| Hs578T | S |
| HT29 | R |
| IGROV1 | R |
| IMR32 | S |
| K562 | R |
| KM12 | R |
| LNCAP | R |
| LNCaPcloneFGC | R |
| LOXIMVI | S |
| M059K | S |
| Malme3M | R |
| MCF7 | R |
| MDAMB231 | R |
| MDAMB435 | R |
| MDAMB435S | R |
| NCIH1184 | S |
| NCIH1299 | S |
| NCIH1339 | R |
| NCIH146 | R |
| NCIH1618 | R |
| NCIH1770 | S |
| NCIH187 | S |
| NCIH1963 | S |
| NCIH209 | S |
| NCIH226 | R |
| NCIH23 | R |
| NCIH322 | R |
| NCIH345 | R |
| NCIH446 | S |
| NCIH460 | R |
| NCIH522 | R |
| NCIH526 | S |
| NCIH69 | R |
| NCIH727 | R |
| NCIH82 | S |
| NIH:OVCAR3 | S |
| OVCAR4 | R |
| OVCAR5 | R |
| OVCAR8 | R |
| PC3 | R |
| RPMI8226 | R |
| SF268 | S |
| SF295 | R |
| SF539 | R |
| SKMEL2 | R |
| SKMEL28 | R |
| SKMEL5 | R |
| SKNAS | S |
| SKNMC | S |
| SKNSH | S |
| SKOV3 | R |
| SNB75 | R |
| SR | R |
| SW620 | R |
| T47D | R |
| TK10 | R |
| TT | R |
| U118MG | R |
| U251MG | R |
| UACC257 | R |
| UACC62 | S |
| UO31 | R |

(S = Sensitive, R = Resistant)

Specifically, the top 100 up- or down-regulated genes from the comparison of infected and resistant cell lines were used to form the SVV100 gene signature panel (Tables 23A-23B below).

TABLE 23A
Up-regulated Genes in the SVV100 Panel
Up-regulated Genes in the SVV100 Panel

ABCG2
ACSM3
AFAP1L2
AIFM2
ANXA3
APOBEC3B
APOBEC3D
APOL1
AREG
ARHGEF16
ASB9
BHLHE40
C19orf33
C1RL
CAPG
CASP10
CCDC69
CELSR1
CTSZ
CYP2S1
DMKN
DRAM1
DSG2
EREG
ETV4
FAM83H
FAM83H-AS1
FBXO27
FIBCD1
FRK
GDF15
GGT1
GJB2
HCP5
HLA-B
HLA-C
HLA-F
HNF1B
ICOSLG
IFI35
IKBKE
IL15RA
IL18
IL6R
IRF5
ISG20
KDELR3
KYNU
LIPH
LRRC8E
LTBR
MCTP2
MICA
MICB
MLKL
MOCOS
NQO1
PDGFB
PHLDA2
PLD1
PNPLA4
PROSER2
PRRG4
PSD4
PYGL
RAB20
RBM47
RHOD
RNF207
SAT1
SDSL
SERPINA1
SGMS2
SH2D3A
SH3TC1
SLC16A5
SLC52A3
SP100
SQRDL
STAT6
STXBP2
TAP1
TCIRG1
TFAP2C
TGFA
TINAGL1
TMBIM1
TMCO4
TMEM144
TMEM92
TNFRSF10A
TNFRSF14
TNFSF10
TNS3
TPD52L1
TRIM47
USP43
UTRN
VAMP8
VDR

TABLE 23B
Down-regulated Genes in the SVV100 Panel
Down-regulated Genes in the SVV100 Panel

ADAM23
ARL10
ATP1A3
ATP8A1
BCL11A
BEX1
BEX4
BRSK1
BSN
CACNA2D1
CADPS
CAND2
CHGA
CHRNB2
CNRIP1
CPT1C
DACH1
DCHS1
DOK6
DPYSL3
EBF1
EFNB3
ELOVL4
FAM155A
FAT3
FLRT2
FOXO6
FSD1
GABRA3
GNAO1
GNB3
GPC2
GPR162
GPR173
IGFBPL1
ISL1
JAKMIP2
JAM3
KIAA1549L
KIF5A
KIF5C
LIN28B
LRCH2
LZTS1
MAP1A
MEIS1
MEX3A
NAP1L3
NCAM1
NCAM1-AS1
NELL2
NFASC
NFATC4
NRN1
NTM
NYNRIN
PDZD4
PDZRN3
PHF21B
PHYHIPL
PKIA
POU3F2
PPM1E
PRDM8
RAB39A
RCOR2
RIMS3
RNF150
ROBO2
RTN1
RUNX1T1
SAMD11
SATB1
SCG3
SCN2A
SCN3A
SEMA6D
SLC4A8
SMAD9
SNAP25
SOX11
SPTBN4
SULT4A1
SYP
SYT11
SYT14
TCF4
TENM4
THY1
TMEFF1
TMSB15A
TRO
TSPYL5
UNC13A
UNC5B
UNC80
WDR17
ZNF354C
ZNF521
ZNF804A

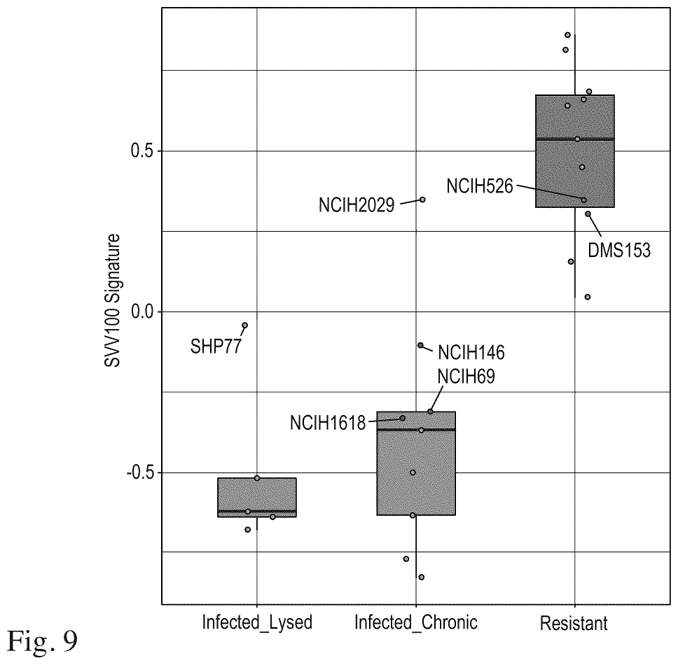

The SVV100 gene signature panel was used to estimate the SVV sensitivity of SCLC cell lines. As shown in FIG. 9, the downregulated genes of the SVV100 panel successfully partitioned the SCLC cell lines according to susceptibility to SVV infection. Notably, the chronically infected SCLC cell lines have intermediate SVV100 signature scores compared to cell lines that are either lytically infected or those that are SVV-resistant, suggesting that the SVV panel captures the range of phenotypes associated with SVV infection.

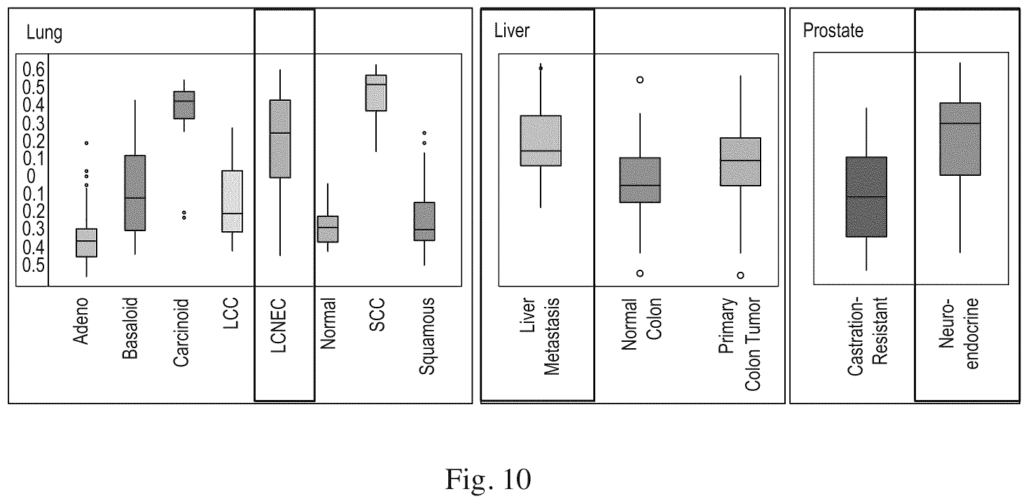

The SVV gene signature panel was then used to estimate the SVV sensitivity of other cancer cells using data reported in Rousseaux et al., Science Translational medicine. 2013 5:186; Kim et al., Molecular Oncology. 2014 8:1653; and Beltran et al, Nat Med. 2016 22(3):298. As shown in FIG. 10, the downregulated genes of the SVV100 panel predicted that many large cell neuroendocrine lung cancers (LCNEC), metastasized liver cancers, and neuroendocrine prostate cancers are also SVV-sensitive.

A similar approach was used to construct the SVV-SCLC gene signature panel (as shown in Tables 10 and 11 above) based on the 20 CCLE cell lines and 14 PDX cell lines as shown in Table 20 above.

### Example 7: Validation of SVV-Sensitivity Prediction Using PDX Models

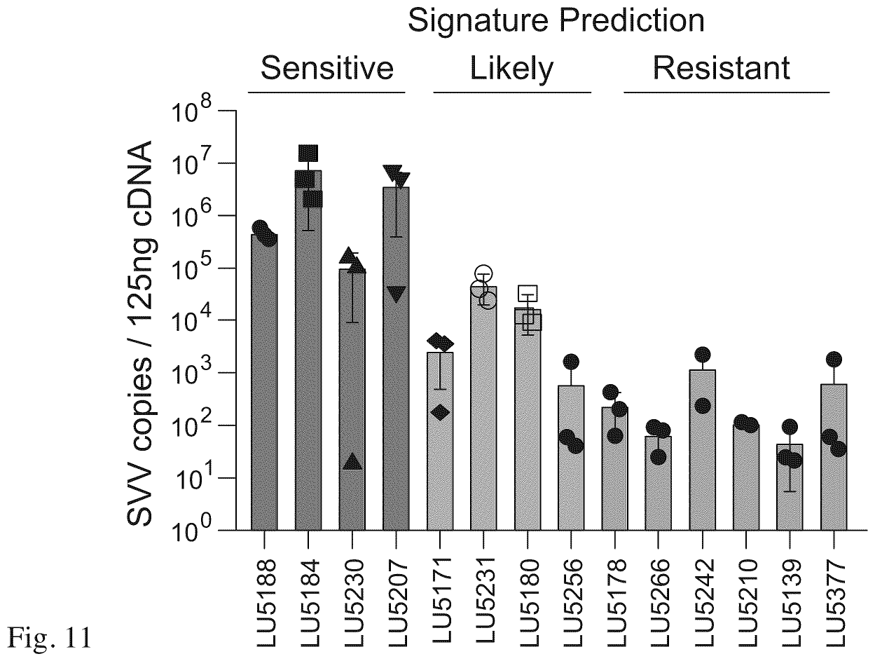

The ELN_1 gene signature panel was used to predict SVV sensitivity for a number of SCLC PDX models, which were classified into three different groups: SVV-sensitive, SVV-likely (lower sensitivity), and SVV-resistant. FIG. 11 shows that the predicted SVV sensitivity aligns well with the replication of SVV in tumors of mice dosed with SVV intratumorally.

Two different PDX models (LU5184 and LU5171) were selected for in vivo efficacy studies. LU5184 was predicted to be SVV sensitive, and LU5171 was predicted to have moderate sensitivity to SVV, based on the gene signature panel analysis. For both PDX models, mice were divided into four treatment groups:
(1) Negative control 1: phosphate buffered saline (PBS).
(2) Negative Control 2: Lipid-nanoparticle comprising negative strand of SVV RNA (SVV mutated sequence that does not lead to viral replication, Synthetic-SVV-Neg).
(3) Lipid-nanoparticle comprising functional SVV viral RNA (Synthetic-SVV).
(4) SVV virus (SVV).

The dosage schedule is shown according to Table 24 below.

TABLE 24
Dosage Schedule of SVV for PDX Models

| | | | Dosing | | Dose |
|---|---|---|---|---|---|
| | | | Frequency & | Dose Level | Volume |
| Group | Treatment | Dose Route | Duration | (mg/kg) | (mL/kg) |
| 1 | PBS | Intravenous | Single | N/A | 5 |
| 2 | Synthetic-SVV- | Intravenous | Single | 1 | 5 |
| | NEG | | | | |
| 3 | Synthetic-SVV | Intravenous | Single | 1 | 5 |
| 4 | SVV | Intratumoral | Q3Dx2 doses | 1 × 10⁶ PFU | 50 μL |

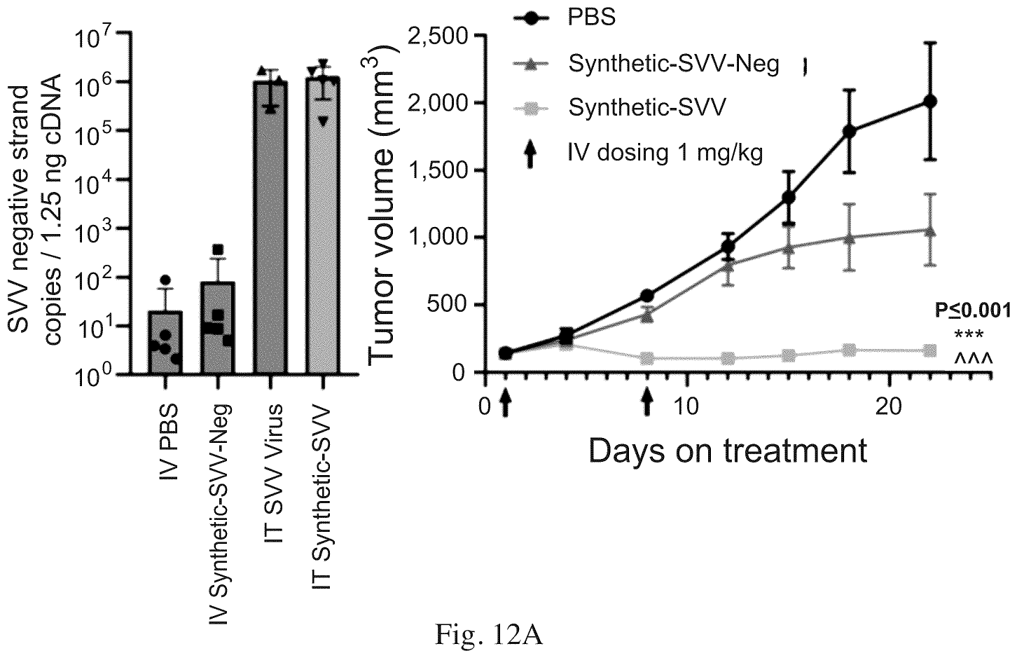

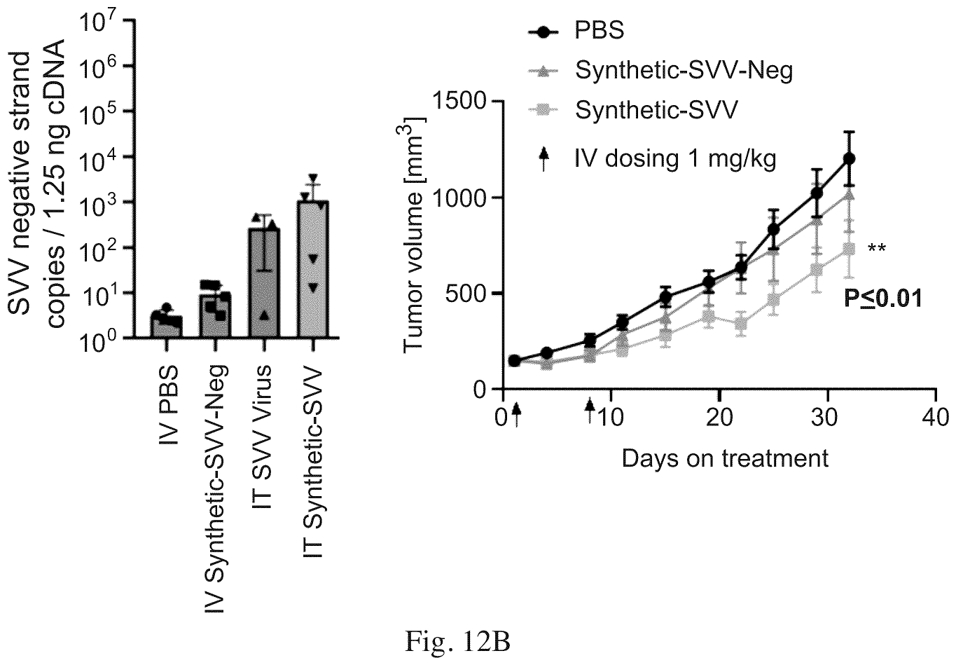

As shown in FIG. 12A, SVV or Synthetic SVV treatment of mice bearing the PDX model that was predicted to be SVV-sensitive (LU5184 PDX model) displayed high viral copies in tumor tissue and significant tumor growth inhibition upon treatment. In contrast, as shown in FIG. 12B, SVV or Synthetic SVV treatment of mice bearing the PDX model that was predicted to be less sensitive to SVV infection (LU5171 PDX model) displayed lower viral copies in tumor tissue and significant but reduced tumor growth inhibition upon treatment. Therefore, the SVV100 panel successfully predicts SVV sensitivity of these PDX models.

### Example 8. Prediction of SVV-Sensitive Prostate Cancer Based on the Gene Signature Panel

The SVV100 gene signature panel was applied to the RNA seq data of human neuroendocrine prostate cancer samples obtained from multiple databases and published literature to predict the percentage and characteristics of SVV-sensitive prostate cancer. Three RNAseq datasets of PDX and tumor biopsy samples of human prostate cancer were used (Labrecque et al., J Clin Invest. 2019 Jul. 30; 129(10):4492-4505; Aggarwal et al., Clin Oncol. 2018 Aug. 20; 36(24):2492-2503; Beltran et al, Nat Med. 2016 March; 22(3):298-305). According to Table 25 below, the gene signature panel predicts that 64-100% of neuroendocrine-positive (NE+) metastatic castration-resistant prostate cancer (mCRPC) is SVV-sensitive, suggesting that the NE+mCRPC is particularly suitable for SVV related treatment.

TABLE 25
Prediction of SVV-Sensitivity for Prostate Cancers

| Tumor | | PDX | | Tumor | | | Tumor | |
|---|---|---|---|---|---|---|---|---|
| Labrecque et al., | | | | Aggarwal et al., 2018; | | | Beltran et al., | |
| 2019 | | | | 2019 | | | 2016 | |
| NE+ | NE− | NE+ | NE− | NE+ | NE+/− | NE− | NE+ | NE− |
| 81% | 10% | 100% | 7% | 64% | 29% | 52% | 77% | 0% |

### Example 9: Prediction of SVV-Sensitive Skin Cancer Based on the Gene Signature Panel

The SVV100 gene signature was applied to the RNA seq data of human skin cancer obtained from multiple databases to predict the percentage and characteristics of SVV-sensitive skin cancer. Two publicly available datasets were identified for skin cancer mRNA profiles: GSE396121 (from PMID: 23223137) and GSE223962 (from PMID: 21422430).

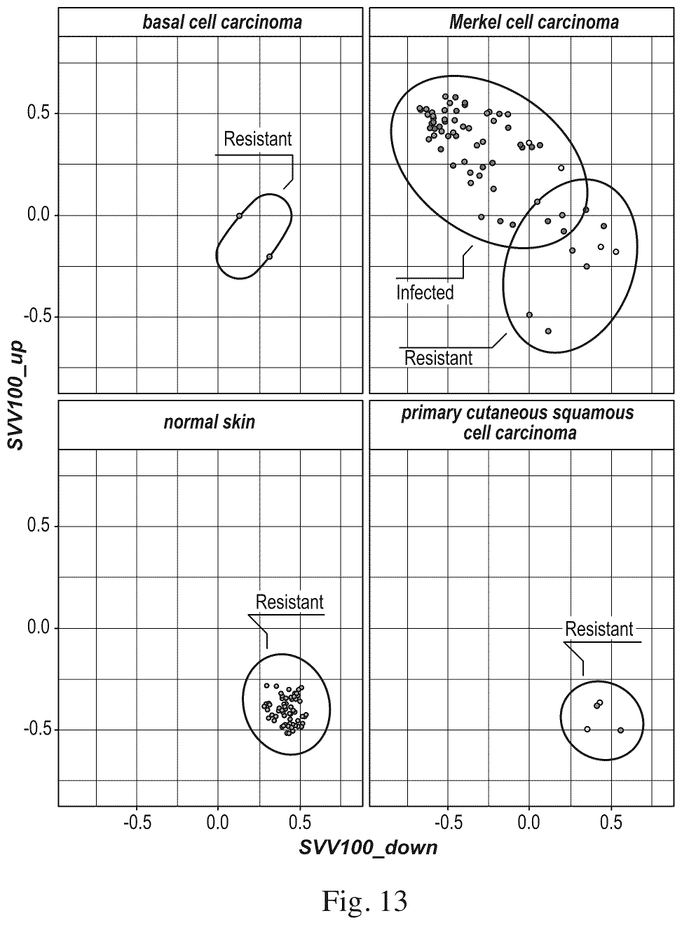

In the first dataset, GSE39612, 138 samples were profiled on the Affymetrix U133_plus_2 chip. The 138 samples represent 2 basal cell carcinomas, 4 primary cutaneous squamous cell carcinomas, 64 normal skin samples, and 68 Merkel Cell Carcinoma samples. As shown in FIG. 13, about 84% of the MCC samples were predicted to be SVV-sensitive. On the other hand, all the normal cell samples and non-MCC samples were predicted to be SVV resistant.

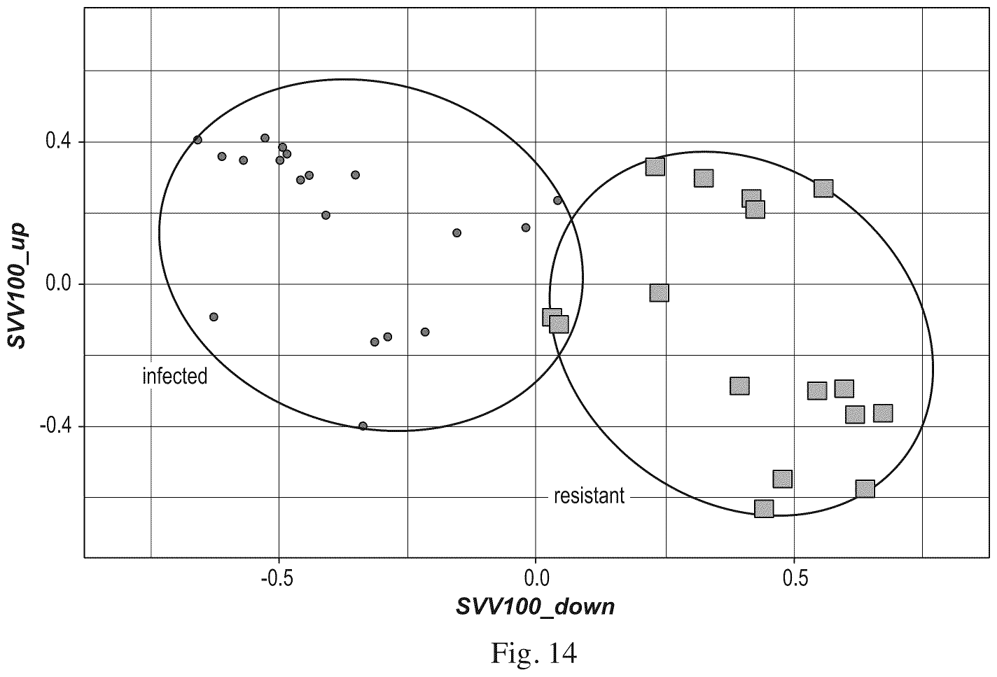

In the second dataset, GSE22396, 35 samples were profiled on the Rosetta/Merck Human RSTA Custom Affymetrix 2.0 microarray. The 35 samples represent both primary and metastatic Merkel Cell Carcinomas. As shown in FIG. 14, about 54% of the MCC samples were predicted to be SVV sensitive.

Taken together, the gene signature panel predicts that 54-84% of Merkel Cell Carcinoma (MCC) is SVV-sensitive.

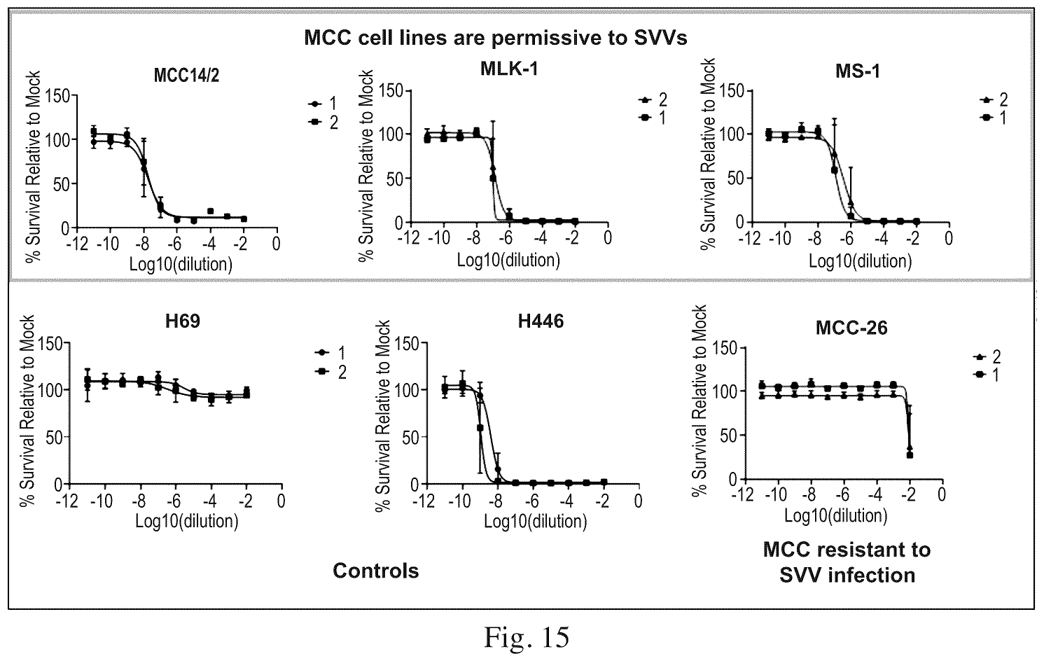

The SVV sensitivity of MCC were then evaluated in vitro. As shown in FIG. 15, among the MCC cell lines tested, 3 cell lines (MCC14/2, MLK-1, and MS-1) are SVV sensitive, whereas the other MCC-26 cell line is SVV resistant. Overall, the majority of the tested MCC cell lines are SVV sensitive, which is consistent with the prediction of the gene signature panel.

### Example 10: SVV Treatment of SCLC Based on Prediction of Gene Signature Panel

A tumor biopsy sample is obtained from a patient diagnosed with SCLC, and a diagnostic kit is used to obtain the mRNA expression levels of the genes in the ELN28_reduced gene signature panel in the tumor sample. The results are then fed to a computer algorithm which classifies the tumor sample as SVV-sensitive. The patient then receives treatment with lipid nanoparticles comprising SVV viral genome.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating a cancer in a subject in need thereof, comprising administering a therapeutically effective amount of a Seneca Valley Virus (SVV) or a polynucleotide encoding the SVV viral genome to the subject, wherein the cancer is classified as sensitive to SVV infection based on the expression level of one or more genes in the cancer, and wherein the one or more genes comprise at least one of the genes listed in one of Tables 1-14 or a combination thereof.

2. A method of treating a cancer in a subject in need thereof, comprising:
(a) determining the expression level of one or more genes in the cancer;
(b) classifying the cancer as sensitive to Seneca Valley Virus (SVV) infection based on the expression level of the one or more genes as determined in (a); and
(c) administering a therapeutically effective amount of a Seneca Valley Virus (SVV) or a polynucleotide encoding the SVV viral genome to the subject if the cancer is classified as sensitive to SVV infection in step (b),
wherein the one or more genes comprise at least one of the genes listed in one of Tables 1-14 or a combination thereof.

3. A method of evaluating the sensitivity of a cancer to Seneca Valley Virus (SVV) infection, comprising determining the expression level of one or more genes in the cancer, wherein the one or more genes comprise at least one of the genes listed in one of Tables 1-14 or a combination thereof, and classifying the cancer as sensitive or resistant to SVV infection based on the expression level of the one or more genes.

4. (canceled)

5. A method of selecting a subject suffering from a cancer for treatment with a Seneca Valley Virus (SVV) or a polynucleotide encoding the SVV viral genome, comprising:
(a) determining the expression level of one or more genes in the cancer, wherein the one or more genes comprise at least one of the genes listed in one of Tables 1-14 or a combination thereof;
(b) classifying the cancer as sensitive to SVV infection based on the expression level of the one or more genes as determined in (a); and
(c) selecting the subject for treatment with the SVV or the polynucleotide encoding the SVV viral genome if the cancer is classified as sensitive to SVV infection in (b).

6.-10. (canceled)

11. The method of claim 1, wherein the one or more genes have a frequency of at least 10%, 15%, 20%, 25%, 30%, 35%, 40%, 45%, 50%, 55%, 60%, 65%, 70%, 75%, or 80% in Table 2 or 3.

12. The method of claim 1, wherein the one or more genes comprise all genes having a frequency of at least 40% in Table 2 or 3.

13. The method of claim 1, wherein the one or more genes comprise all genes having a frequency of at least 10%, 15%, 20%, 25%, 30%, 35%, 40%, 45%, 50%, 55%, 60%, 65%, 70%, 75%, or 80% in Table 2 or 3.

14. (canceled)

15. The method of claim 1, wherein the one or more genes comprise all genes in one of Tables 2-7, or all genes in Tables 8-9, or all genes in Tables 10-11.

16. The method of claim 15, wherein the one or more genes comprise all genes in Table 3.

17.-29. (canceled)

30. The method of claim 1, wherein the one or more genes comprise at least 2, 3, 4, 5, 6, 7 or 8 genes selected from the group consisting of HLA-C, STAT6, TMCO4, CNRIP1, DCAF13, PRDM8, DACH1, and IKBKE.

31. The method of claim 1, wherein the one or more genes comprise at least one gene selected from the group consisting of HLA-C, STAT6, TMCO4, and CNRIP1.

32. The method of claim 1, wherein the one or more genes comprise 2, 3, or 4 genes selected from the group consisting of HLA-C, STAT6, TMCO4, and CNRIP1.

33. The method of claim 1, wherein the one or more genes comprise at least one gene selected from the group consisting of HLA-C, CNRIP1, JPH1, TMCO4, STAT6, DENND2D, ETV7, DACH1, DCAF13, GLCE, NSMF, SCG3, IKBKE, TNFRSF14, ARHGEF16, MICB, TAP1, USP43, GSDMD, HOXC11, and SMAD7.

34. The method of claim 1, wherein the one or more genes comprise at least one gene selected from the group consisting of HLA-C, CNRIP1, JPH1, TMCO4, STAT6, DENND2D, and ETV7.

35. The method of claim 1, wherein the one or more genes comprise at least 2, 3, 4, 5, 6, or 7 genes selected from the group consisting of HLA-C, CNRIP1, JPH1, TMCO4, STAT6, DENND2D, and ETV7.

36. The method of claim 1, wherein the one or more genes comprise at least one gene selected from the group consisting of HLA-C, CNRIP1, JPH1, and TMCO4.

37. The method of claim 1, wherein the one or more genes comprise 2, 3, or 4 genes selected from the group consisting of HLA-C, CNRIP1, JPH1, and TMCO4.

38. The method of claim 1, wherein the one or more genes comprise HLA-C.

39. The method of claim 1, wherein the one or more genes do not comprise ANTXR1 and do not comprise IFI35.

40.-45. (canceled)

46. The method of claim 1, wherein the polynucleotide is a recombinant RNA molecule, and wherein the polynucleotide encoding is encapsulated in a lipid nanoparticle.

47. (canceled)

48. (canceled)

49. The method of claim 1, wherein the expression level of the one or more genes is mRNA expression level or protein expression level.

50. (canceled)

51. The method of claim 1, wherein the expression level of the one or more genes is protein expression level.

52. (canceled)

53. (canceled)

54. The method of claim 1, wherein the cancer is a neuroendocrine cancer or small cell lung cancer (SCLC).

55. (canceled)

56. The method of claim 1, wherein the neuroendocrine cancer is neuroendocrine-positive (NE+) metastatic castration-resistant prostate cancer (mCRPC).

57. (canceled)

58. The method of claim 54, wherein the SCLC is NeuroD1+SCLC.

59.-69. (canceled)

70. A kit, comprising reagents for determining the expression level of one or more genes in a sample from a subject in need of, wherein the one or more genes comprise any one of the genes listed in one of Tables 1-14 or a combination thereof.

71.-115. (canceled)
